(19) United States
(12) Patent Application Publication
(10) Pub. No.: US 2023/0047825 A1
(43) Pub. Date: Feb. 16, 2023

(54) ANTIMICROBIAL PEPTIDES

(71) Applicants: KING'S COLLEGE LONDON, London (GB); SECRETARY OF STATE FOR HEALTH AND SOCIAL CARE, London (GB)

(72) Inventors: John Mark SUTTON, Salisbury Wiltshire (GB); Andrew James MASON, London (GB); Richard Thomas AMISON, London (GB)

(21) Appl. No.: 17/781,907

(22) PCT Filed: Dec. 1, 2020
(86) PCT No.: PCT/GB2020/053078

§ 371 Date: Jun. 2, 2022

(30) Foreign Application Priority Data
Dec. 3, 2019 (GB) 1917644.5
Oct. 19, 2020 (GB) 2016542.9

(51) Int. Cl. A61K 38/17; C07K 14/46; A61P 31/04

(52) U.S. Cl. CPC A61K 38/17; C07K 14/46; A61P 31/04

(57) ABSTRACT

The invention relates to antimicrobial peptides (AMPs). The invention also relates to uses, methods of treatment, pharmaceutical compositions and combinations with antimicrobial agents.

### FIELD OF THE INVENTION

The invention relates to antimicrobial peptides (AMPs).

### BACKGROUND

Antimicrobial resistance (AMR) is one of major threats facing global health today.

New resistance mechanisms are emerging and spreading globally, threatening our ability to treat common infectious diseases, resulting in prolonged illness, disability, and death. Without effective antimicrobials for prevention and treatment of infections, medical procedures such as organ transplantation, cancer chemotherapy, diabetes management and major surgery (for example, caesarean sections or hip replacements) become very high risk. Antimicrobial resistance increases the cost of health care with lengthier stays in hospitals and more intensive care required.

According to the World Health Organisation (WHO), resistance in Klebsiella pneumoniae (a common intestinal bacterium that can cause life-threatening infections) to the last resort treatment of carbapenem antibiotics has spread to all regions of the world. K. pneumoniae is a major cause of hospital-acquired infections such as pneumonia, bloodstream infections, and infections in newborns and intensive-care unit patients. In some countries, because of resistance, carbapenem antibiotics do not work in more than half of people treated for K. pneumoniae infections, which is a serious problem in the art. Resistance in E. coli to one of the most widely used antibiotic for the treatment of urinary tract infections (fluoroquinolone antibiotics) is very widespread. There are countries in many parts of the world where this treatment is now ineffective in more than half of patients, which is a problem in the art. Treatment failure of the last resort treatment for gonorrhoea (third generation cephalosporin antibiotics) has been confirmed in at least 10 countries (Australia, Austria, Canada, France, Japan, Norway, Slovenia, South Africa, Sweden and the United Kingdom of Great Britain and Northern Ireland), which is a significant problem. Resistance to first-line drugs to treat infections caused by Staphlylococcus aureus—a common cause of severe infections in health facilities and the community—is widespread. People with MRSA (methicillin-resistant Staphylococcus aureus) are estimated to be 64% more likely to die than people with a non-resistant form of the infection, which is a major problem. Colistin is the last resort treatment for life-threatening infections caused by Enterobacteriaceae which are resistant to carbapenems. Resistance to colistin has recently been detected in several countries and regions, making infections caused by such bacteria untreatable. Thus human lives are being lost due to problems in the art AMR is further complicated due the bacterial biofilm forming capability. Thus, bacterial infections are becoming increasingly difficult to treat with conventional antibiotics present in clinics. There is ongoing research into the development of novel antibacterial agents to tackle this challenging problem. Recently, antimicrobial peptides (AMPs) which are also known to display antibiofilm capability in some cases, have shown promise.

Antimicrobial peptides (AMPs) provide an attractive alternative to traditional small molecule antibiotics to fight antimicrobial resistance. AMPs are a component of the innate immune system involved in fighting off infections and have been isolated from almost all creatures. They often have potent antimicrobial activity. However, AMPs are subject to degradation by serum proteases and may be poorly tolerated in murine models. As such, no AMPs have yet been successfully used to treat systemic infections. This is a problem in the art.

A number of antimicrobial peptides from different classes have been examined as potential therapeutic agents. Linear antimicrobial peptides that have reached Phase III clinical trials are typically confined to topical applications e.g. treating infections of diabetic foot ulcers (Locilex®; pexiganan acetate) or acne vulgaris/papulopustular rosacea (CLS001; omiganan pentahydrochloride). Currently therefore the indications that antimicrobial peptides may address are limited and therapy for a large number of bacterial infections, for which systemic delivery is required, is inaccessible.

There are approximately 36 antimicrobial peptides (AMPs) in clinical development. Of these, only 6 are systemic—the overwhelming majority of AMPs in clinical development remain topical. This is a problem in the art. Moreover, none of the 36 AMPs in clinical development are thought to act in a bactericidal manner. Prior art approaches have tended to focus on immunostimulation.

One of the better known prior art AMPs is Pexaganan (a derivative of Magainin). This has been used as a possible treatment for diabetic foot ulcers. This involved topical application. This prior art use failed.

The view in the art is that degradation of AMPs is expected. The view in the art is that AMPs are likely to produce harmful immune responses. These are problems in the art.

The present invention seeks to overcome problem(s) associated with the art.

### SUMMARY

The inventors have gone against the thinking in the art. The inventors have worked to produce systemic AMPs. Moreover, the inventors teach the new use of AMPs as direct antibacterial agents. As noted above, prior art uses had focused on immunostimulation, therefore the inventors' teaching to use AMPs as direct antibacterial agents is itself a novel approach.

The inventors embarked upon an intensive and challenging research program. Numerous peptide designs were tried but showed insufficient activity. The inventors had inspiration to start from a known peptide pleurocidin. In the course of their research, the inventors took intellectual decisions leading them to introduce a unique property of conformational flexibility into the molecules by changing the starting molecule (pleurocidin) to create new peptide sequences. This is fully unexpected and very surprising to the inventors, because the view in the art is that most alpha helical peptides (such as pleurocidin) stay alpha helical. However, the inventors have produced an alpha helical peptide which displays conformational flexibility, which appears to be a new and unique property.

The inventors teach numerous novel advantageous peptides delivering the same technical advantage(s) based on the pleurocidin peptide sequence. In addition, the inventors demonstrate the ability of a fully characterised synthetic derivative of a natural AMP to treat systemic infections in a murine model.

The invention is based upon these surprising findings.

Thus in one aspect the invention provides a peptide comprising amino acid sequence having at least 52% sequence identity to SEQ ID NO: 1, wherein said amino acid sequence comprises one or more of the following substitutions relative to SEQ ID NO: 1:
i. K7R
ii. K8R
iii. V12A or V12I or V12L
iv. K14R
v. V16A or V16I or V16 L
vi. K18R.

In another embodiment the invention relates to a peptide as described above comprising at least the following substitutions relative to SEQ ID NO: 1:
i. K7R
ii. K8R
iv. K14R; and
vi. K18R.

In another embodiment the invention relates to a peptide as described above comprising at least the following substitutions relative to SEQ ID NO: 1:
iii. V12A or V12I or V12L
v. V16A or V16I or V16 L.

In another embodiment the invention relates to a peptide as described above comprising at least the following substitutions relative to SEQ ID NO: 1:
i. K7R, and
ii. K8R, and
iii. V12A or V12I or V12L, and
iv. K14R, and
v. V16A or V16I or V16 L; and
vi. K18R.

In another embodiment the invention relates to a peptide as described above further comprising one or more of the following substitutions relative to SEQ ID NO: 1:
a) F5Y or F5W
b) F6Y or F6W
c) substitution of H11 for 3-methylhistidine or 1-methylhistidine or 2,3-diaminopropionic acid or (N-methyl or N,N-dimethyl derivative of 2,3-diaminopropionic acid) or E or D;
d) substitution of H15 for 3-methylhistidine or 1-methylhistidine or 2,3-diaminopropionic acid or (N-methyl or N,N-dimethyl derivative of 2,3-diaminopropionic acid) or E or D
e) substitution of H23 for 3-methylhistidine or 1-methylhistidine or 2,3-diaminopropionic acid or (N-methyl or N,N-dimethyl derivative of 2,3-diaminopropionic acid) or E or D.

In another embodiment the invention relates to a peptide as described above wherein one or more of amino acids 9 to 21 is substituted for N-substituted glycine.

In another embodiment the invention relates to a peptide as described above wherein said peptide comprises amino acid sequence having at least 76% sequence identity to SEQ ID NO: 1.

In another embodiment the invention relates to a peptide as described above wherein said peptide is at least 25 amino acids in length, suitably wherein said peptide is 25 amino acids in length.

In another embodiment the invention relates to a peptide comprising amino acid sequence selected from SEQ ID NO: 2 or SEQ ID NO: 3 or SEQ ID NO: 4, suitably consisting of amino acid sequence selected from SEQ ID NO: 2 or SEQ ID NO: 3 or SEQ ID NO: 4.

In one embodiment the invention relates to a peptide as described above consisting of L-amino acids.

In another embodiment the invention relates to a peptide as described above comprising one or more D-amino acids. More suitably the invention relates to a peptide as described above consisting of D-amino acids.

In another embodiment the invention relates to a peptide as described above for use in medicine.

In another embodiment the invention relates to a peptide as described above for use in treatment or prevention of infection, suitably bacterial infection.

In another embodiment the invention relates to a peptide as described above for use as an antimicrobial.

In another embodiment the invention relates to a peptide as described above for use as an antibacterial.

In another embodiment the invention relates to use of a peptide as described above as an antimicrobial.

In another embodiment the invention relates to use of a peptide as described above as an antibacterial.

In another embodiment the invention relates to a pharmaceutical composition comprising a peptide as described above.

Suitably said pharmaceutical composition further comprises an antibiotic agent.

In another embodiment the invention relates to a method of treating or preventing infection, suitably bacterial infection, in a subject comprising administering a peptide as described above to said subject.

Suitably said method further comprises administering an antibiotic agent to said subject.

In another embodiment the invention relates to a pharmaceutical composition as described above or a method as described above wherein said antibiotic agent comprises one or more of colistin, tobramycin and rifampin.

Suitably the antibiotic agent is colistin or tobramycin, suitably tobramycin.

In another embodiment the invention relates to a pharmaceutical composition comprising a peptide and an antibiotic agent, wherein said peptide and said antibiotic agent are selected from a single row of Table 3.

In another embodiment the invention relates to a pharmaceutical composition as described above or a method as described above, wherein the bacterial infection is P.aeruginosa infection or A.baumannii infection, suitably P.aeruginosa infection.

In another embodiment the invention relates to a pharmaceutical composition as described above or a method as described above, wherein the peptide comprises the amino acid sequence of SEQ ID NO:2, and consists of D-amino acids.

### DETAILED DESCRIPTION OF THE INVENTION

It is demonstrated herein that it is possible to generate stable and readily soluble rationally designed analogues of a natural AMP with differential specificity for Gram-negative and Gram-positive pathogens, which can be delivered intravenously to treat bacterial lung infections. The absence of cytokine release or immune cell recruitment suggests that this may be the first example of effective systemic AMP treatment of a lung infection mediated by direct bacterial killing.

The inventors took the intellectual decision to begin from pleurocidin, a potent AMP found in the Winter Flounder, rather than any of the other many and varied known AMPs. When compared with other AMPs with apparent preference for α-helix conformation, pleurocidin has much greater conformational flexibility and the inventors decided to use this as a starting point as they believed it may enable better penetration of bacteria e.g. to reach intracellular targets.

Pleurocidin analogues are described.

New AMPs are disclosed. The inventors designed them with reference to pleurocidin. They conceived mutations which they introduced into the pleurocidin peptide sequence. The inventors assert that none of these important mutations have been made before.

As is explained in more detail below, in preferred embodiments the inventors have made substitutions which retained the same basic properties in the amino acids used, and retained the same pKa of the resulting peptide. However, among the key differences which the inventors have consciously engineered into the peptides are their altered hydrogen bonding properties. In particular, the inventors have targeted the ‘alpha helix versus membrane interaction’ balance by manipulating the hydrogen bonding properties of the peptide.

This intellectual difference in approach is actually contrary to what might be expected from the prior art. For example, when making a V to A substitution, in the prior art this is regarded as conservative; in the prior art people try to increase the positive charge on the polypeptide; in the prior art people try to increase the hydrophobicity of these peptides (pleurocidin being a particularly hydrophilic peptide). Thus, the V to A substitutions go directly against prior art approaches.

Considering the K to R substitutions, the view in the art is that these would disrupt the alpha helical structure; the view in the art is that disrupting the alpha helical structure makes the polypeptide less active. Document(s) published before the priority date clearly establish this prior art view, such as Cuervo et al 1988 (see below). The fact that the inventors made mutations going against the current thinking in the art is indicative of inventive step/non-obviousness.

In the prior art the view is to try to eliminate negatively charged amino acids. The inventors have surprisingly gone against this prejudice in the art in arriving at the peptides of the invention disclosed herein.

It is important to note that the novel peptides disclosed herein are not simply “different” or mere alternatives to existing reagents—in fact they possess superior technical benefits which further establish inventive step/non obviousness. For example, we demonstrate herein enhanced effects on membrane disruption, and we demonstrate increased potency of the peptides, and we demonstrate an enhanced therapeutic index.

In addition, we demonstrate a higher potency, particularly against Gram negative bacteria (in the prior art antimicrobial peptides have tended to be more active against Gram positive bacteria). Thus experimental evidence demonstrating the new and improved qualities of the new peptides of the invention is provided. This is discussed in more detail below and in the accompanying drawings.

When used herein, unless otherwise apparent from the context, relative terms such as “more potent”, “greater membrane damage”, “greater conformational flexibility”, “higher potency”, “greater membrane disruptive effect” (and any other relative terms) mean by comparison to the pleurocidin reference peptide (SEQ ID NO: 1). Thus “more potent” means more potent than the pleurocidin reference peptide (SEQ ID NO: 1); “greater membrane damage” means greater membrane damage than the pleurocidin reference peptide (SEQ ID NO: 1) etc. In case of any doubt, the skilled reader can easily test the property being discussed and compare the value to the value obtained from the same test using the pleurocidin reference peptide (SEQ ID NO: 1) in place of the peptide of interest; by comparing the values it is immediately apparent if the peptide of interest has (for example) “greater conformational flexibility” i.e. greater conformational flexibility than the pleurocidin reference peptide (SEQ ID NO: 1).

“Strong” has its usual meaning as apparent from the context. In some embodiments, “strong” means an FICₘᵢₙ of 0.3 or less.

“Infection” means bacterial infection unless otherwise apparent from the context.

“Antibiotic agent” refers to conventional known antibiotics. These are typically non-peptide compounds. Suitably the term ‘antibiotic agent’ as used herein excludes peptide compounds unless otherwise apparent from the context. Antibiotic agent refers to organic compound(s) such as small organic compounds. Exemplary classes of antibiotic agent (i.e. conventional known antibiotics) include carbapenem antibiotics, aminoglycoside antibiotics, cephalosporin antibiotics and the like.

We describe rationally designed analogues of pleurocidin. Modifications to the natural sequence have been made that increase conformational disorder. These modifications either increase antibacterial potency or reduce host cell toxicity. The modifications alter the mechanism of action and confer serum/protease stability. In vivo efficacy has been demonstrated (i.v. delivery to treat murine EMRSA-15 lung infection). Combinations of pleurocidin (and its analogues) such as the peptides of the invention with classical antibiotics are described. In vitro data for combinations with tobramycin against Gram-negative pathogens are provided—these show a 10-fold reduction in AMP and tobramycin required for the same effect.

### Surprising Potency

The inventors have identified that conformational flexibility is a peculiar property of pleurocidin when binding to models of bacterial plasma membranes. Here this is shown to increase in two new especially suitable analogues. It is disclosed that in one exemplary AMP, (D)-pleurocidin-KR, this is linked to increased antibacterial potency. Without wishing to be bound by theory, the increased conformational flexibility in this analogue may contribute to the increased potency, however this is not the main factor. Instead the increased hydrogen bonding capability of the new analogue leads to greater membrane damage. This also appears to contribute a further technical advantage of a reduced ability to translocate. The new mechanism of action achieved by the molecules disclosed herein has further advantages such as being more robust to changes in the bacterial metabolic strategy. This property is beneficial to underpin hit-to-lead development where in vivo potency can be expected even if the metabolic strategy of the bacteria in each infection setting is unknown.

The inventors teach that this technical effect may be achieved in other ways and it may be possible to achieve this technical effect without completely compromising the translocation capability of the parent by (for example):
1. Permutation of the four lysine to arginine substitutions (only three, two or even one substitution may achieve a beneficial effect); and/or
2. Substitution of phenylalanine(s) with tyrosine or tryptophan residues.

Various permutations (i.e. possible combinations of substitutions taught herein) are illustrated in the table of examples below (Table P) in order to aid understanding. The disclosure is not to be understood as limited to these examples.

TABLE P

| Exemplary Peptide | Exemplary Sequence | SEQ ID NO: |
|---|---|---|
| Pleurocidin | GWGSFFKKAAHVGKHVGKAALTHYL-NH₂ | SEQ ID NO: 1 |
| (WF2/NRC-04) | | (reference sequence) |
| Pleurocidin-KR | GWGSFFRRAAHVGRHVGRAALTHYL-NH₂ | SEQ ID NO: 4 |
| Pleurocidin-KR - per1 | GWGSFFRRAAHVGRHVGKAALTHYL-NH₂ | SEQ ID NO: 5 |
| Pleurocidin-KR - per2 | GWGSFFRRAAHVGKHVGRAALTHYL-NH₂ | SEQ ID NO: 6 |
| Pleurocidin-KR - per3 | GWGSFFRRAAHVGKHVGKAALTHYL-NH₂ | SEQ ID NO: 7 |
| Pleurocidin-KR - per4 | GWGSFFKKAAHVGRHVGRAALTHYL-NH₂ | SEQ ID NO: 8 |
| Pleurocidin-KR - per5 | GWGSFFRKAAHVGRHVGRAALTHYL-NH₂ | SEQ ID NO: 9 |
| Pleurocidin-KR - per | GWGSFFKRAAHVGRHVGRAALTHYL-NH₂ | SEQ ID NO: 10 |
| Pleurocidin-KR - per7 | GWGSFFKRAAHVGRHVGKAALTHYL-NH₂ | SEQ ID NO: 11 |
| Pleurocidin-KR - tran1 | GWGSYYRRAAHVGRHVGRAALTHYL-NH₂ | SEQ ID NO: 12 |
| Pleurocidin-KR - tran2 | GWGSWWRRAAHVGRHVGRAALTHYL-NH₂ | SEQ ID NO: 13 |
| Pleurocidin-KR - tran3 | GWGSYWRRAAHVGRHVGRAALTHYL-NH₂ | SEQ ID NO: 14 |
| Pleurocidin-KR - tran4 | GWGSWYRRAAHVGRHVGRAALTHYL-NH₂ | SEQ ID NO: 15 |

It is to be noted that the increase in conformational flexibility runs counter to the paradigm where increased α-helix conformation is predicted to correlate with increased antibacterial potency, hence the prior art teaches away from the method of the invention (see Cuervo, J. H., Rodriguez, B. & Houghten, R. A. (1988) The Magainins: sequence factors relevant to increased antimicrobial activity and decreased hemolytic activity. Pept. Res. 1, 81-6; and/or see Dathe, M., and Wieprecht, T. (1999) Structural features of helical antimicrobial peptides: their potential to modulate activity on model membranes and biological cells. Biochim. Biophys. Acta 1462, 71-87.). Thus it is established that the new approach disclosed in this invention is surprising to a person skilled in the art.

The inventors have used proline in synthetic peptides to show that it is important where the conformational flexibility is located (see Vermeer, L. S., Lan, Y., Abatte, V., Ruh, E., Bui, T. T., Wilkinson, L., Kanno, T., Jumagulova, E., Kozlowska, J., Patel, J., McIntyre, C. A., Yam, W. C., Siu, G., Atkinson, R. A., Lam, J. K. W., Bansal, S. S., Drake, A. F., Mitchell, G. H. & Mason, A. J. (2012)* Conformational flexibility determines selectivity and antibacterial, antiplasmodium and anticancer potency of cationic α-helical peptides. J. Biol. Chem. 287, 34120-34133). Such flexibility is however commonly understood to be associated with proline or glycine “helix breaking” residues and not to other amino acids that contribute to the overall interaction with a membrane interface region. Thus the teachings presented herein are new and are not suggested in the prior art. In fact, the teachings presented herein are surprising and go against the teachings known in the art.

### Advantageous Selectivity

One exemplary D-pleurocidin-VA peptide has two valine to alanine substitutions (V12A and V16A) near two glycine residues that confer advantageous properties such as conformational flexibility, and/or how the peptide is able to approach and/or orient various residues at a membrane surface. Further technical effects delivered by this structural feature include that the resulting peptide has much lower in vitro cellular toxicity—this may additionally improve to the maximum tolerated dose in vivo.

Potency against many isolates is reduced but retained against e.g. Acinetobacter baumannii, one of the WHO Priority pathogens and hence this property is clearly useful in providing better tolerated peptides and/or providing peptides with a more focused spectrum of activity.

The inventors teach that these valine residues, and others nearby, are believed to affect the ability of three histidine residues to interact with the surface. Manipulation of the histidine residues, or other residues nearby, may achieve the same or a similar effect to the double or single valine to alanine substitutions, or in another embodiment may mitigate their effect, enabling a peptide with greater selectivity and/or potency to be generated, more suitably enabling a peptide with greater selectivity and potency to be generated.

Examples of technical features delivering these effect(s) include, but are not limited to:
1) Substitution of only one valine (either Val12 or Val16) or of both valines (both Val12 and Val 16), suitably with alanine or, in another embodiment, one or more other amino acid(s) or, in another embodiment, an amino acid selected from the group consisting of alanine, isoleucine or leucine.
   In more detail, these substitutions act to affect the protonation state and/or the ability of histidine residues (His11, His15 and His23) to form hydrogen bonds with zwitterionic and/or anionic lipids in bacterial membranes. For example, leucine may be used instead of alanine (i.e. a V12L and/or a V16L substitution instead of a V12A and/or a V16A substitution); substitution of valine with leucine has the technical effect of being more hydrophobic but favours extended conformations less than valine. If alanine or leucine is used to replace only one of Val12 or Val16 then the remaining valine may be substituted with isoleucine (i.e. a V12I and/or a V16I substitution, more suitably combination substitutions of V12A V16I, or V12L V16I, or V12I V16A, or V12I V16L); this has the technical benefit of balancing this effect (greater hydrophobicity still and also favours extended conformations). Substituting either valine with isoleucine (in the absence of other modifications) would be expected to either have modest/no effect or to reduce selectivity.
   Various permutations (i.e. possible combinations of substitutions taught herein) are illustrated in the table of examples below (Table V) in order to aid understanding. The disclosure is not to be understood as limited to these examples. The V12 and V16 positions are underlined.

TABLE V

| Exemplary Peptide | Exemplary Sequence | SEO ID NO: |
|---|---|---|
| Pleurocidin | GWGSFFKKAAHVGKHVGKAALTHYL-NH₂ | SEQ ID NO: 1 |
| (WF2/NRC-04) | | (reference sequence) |
| Pleurocidin-VA | GWGSFFKKAAHAGKHAGKAALTHYL-NH₂ | SEQ ID NO: 16 |
| Pleurocidin-V16A | GWGSFFKKAAHVGKHAGKAALTHYL-NH₂ | SEQ ID NO: 17 |
| Pleurocidin-V12A | GWGSFFKKAAHAGKHVGKAALTHYL-NH₂ | SEQ ID NO: 18 |
| Pleurocidin-VAI | GWGSFFKKAAHAGKHIGKAALTHYL-NH₂ | SEQ ID NO: 19 |
| Pleurocidin-VIA | GWGSFFKKAAHIGKHAGKAALTHYL-NH₂ | SEQ ID NO: 20 |
| Pleurocidin-VL | GWGSFFKKAAHLGKHLGKAALTHYL-NH₂ | SEQ ID NO: 21 |
| Pleurocidin-V12L | GWGSFFKKAAHLGKHVGKAALTHYL-NH₂ | SEQ ID NO: 22 |
| Pleurocidin-V16L | GWGSFFKKAAHVGKHLGKAALTHYL-NH₂ | SEQ ID NO: 23 |

2) Use of a peptoid (N-substituted glycine) backbone in the hinge region—peptoids lack the amide hydrogen and hence amide planarity required for ordered conformations.
   In more detail, use of N-substituted glycine in place of any of the α-amino acids between Ala9 and Leu21 has the technical benefit of affecting the conformational flexibility and/or orientation and hydrogen bonding potential of the one or more of the three histidine residues.
3) Use of 3-methylhistidine (or 1-methylhistidine) in place of one or more histidine residue(s)—the pKa of 3-methylhistidine is 6.47 and hence higher than that of histidine (6.01) and so the ability of the peptide to hydrogen bond with lipids in this region may thereby be increased.
4) Use of 2,3-diaminopropionic acid (or N-methyl or N,N-dimethyl derivatives thereof) in place of one or more histidine(s) has the advantage of producing peptides with side chains with a raised pKa and also altered hydrogen bonding potential (primary, secondary or tertiary amines as opposed to imidazole).
5) Replacement of one or more histidine residues with glutamate or aspartate—these residues have a pKa of approximately 4 (respectively 4.07 and 3.90) which will be raised when located near to negatively charged groups (such as are found in bacterial membranes). While these residues are expected to be negatively charged for D-pleurocidin analogues in solution, they may be protonated and hence neutral (and more hydrophobic) when binding to bacterial membranes. Such a substitution is believed to offer the advantage of increased selectivity.

The invention relates to peptides having one or more of the structural features outlined above. More suitably the invention relates to peptides having two or more of the structural features outlined above in combination.

### Synergy

Aminoglycoside antibiotics are a class of antibiotics. Suitably the antibiotic is an aminoglycoside antibiotic. The inventors teach that this is a particular advantage of the combination (rather than a generic effect e.g. where D-pleurocidin and its analogues simply increase permeability to classical antibiotics). Thus suitably the invention relates to use of a combination of an AMP as described herein and an antibiotic such as an aminoglycoside antibiotic in the prevention or treatment of infection such as bacterial infection.

Synergy is observed between the aminoglycoside antibiotic tobramycin and each of the three exemplary D-pleurocidin analogues disclosed herein. This is especially advantageous as it allows reduction of the dose of antibiotic such as Tobramycin which is administered to a subject. This is particularly beneficial as antibiotic such as Tobramycin treatment is challenging for patient compliance because it can produce a range of unpleasant and uncomfortable side effects and so any reduction in the dose of antibiotic such as Tobramycin is clinically very beneficial.

Without wishing to be bound by theory, the exact mechanisms of synergy may differ between particular suitable analogues or AMPs according to the present invention (such as the preferred two exemplary analogues disclosed) and the D-pleurocidin parent. The differing mechanisms may also be affected by the nutritional environment and hence metabolic strategy of the bacteria—this will affect the properties of the most preferred D-pleurocidin analogue(s) and their success in vivo.

### Hydrogen Bonding

Suitably the peptides disclosed herein which enhance hydrogen bonding within the peptide are most preferred.

Most suitably, the peptides described herein which enhance the interaction of the peptide with the bacterial membrane due to hydrogen bonding are most preferred.

### Applications

The inventors created a series of antimicrobial peptides derived from the winter flounder. We present data showing the activity of various peptides and demonstrate broad spectrum activity in the sub-μM range against Gram-negative bacteria (including A. baumannii, K.pneumoniae, E. coli and P. aeruginosa) and also show some activity against two key MDR Gram-positive species (S.aureus inc MRSA and Enterococcus faecalis/faecium inc VRE).

The inventors undertook an activity-informed optimisation study on pleurocidin which has reduced the MICs for some of the Gram-negative target species, reduced the protease/serum sensitivity of the peptide and extended the spectrum of effective activity against Gram-positive species. The latter, in particular, was an unexpected consequence of the optimisation process and another indicator of inventive step/non-obviousness. The effectiveness of the peptides of the invention is demonstrated in an in vivo animal study, where a reduction in the viable count of bacteria in the lung is shown, and this correlated with a reduction in weight loss and in neutrophil counts; these effects are all indicative of enhanced clearance of the pathogen (MRSA). This data is exceptional as the treatment was given intravenously, demonstrating the potential for systemic delivery of the peptide of the invention and showing its therapeutic effect.

In a preferred embodiment, a modified derivative of pleurocidin (termed D-pleurocidin; DP) selected from one of the optimised variants (e.g. DPVA or DPKR) is useful to treat lung infection caused by a multidrug resistant pathogen, selected from a group that includes one of the following pathogens (Gram-negative bacteria; A. baumannii, K. pneumoniae, E. coli or P. aeruginosa, particularly strains expressing carbapenemases and/or strains which are resistant to 3^{rd} generation cephalosporins and colistin, and Gram-positive; S. aureus including methicillin-resistant strains (MRSA) and Enterococcus faecalis/faecium including vancomycin resistant strain (VRE)).

Suitably the subject has a lung infection.

Suitably the subject has a long term chronic condition (e.g. cystic fibrosis or chronic obstructive pulmonary disease (COPD), suitably with exacerbation(s) such as infection e.g. bacterial infection. Suitably the subject may be in intensive care or may be in hospital. Suitably the subject may have ventilator associated pneumonia (VAP). Suitably the subject may have an infection via acquisition in the community. Suitably the subject may have community associated pneumonia (CAP).

Different particular peptides of the invention may be selected for particular different species of bacterial infection. Such decisions are typically made by the physician.

Suitably the infection is an infection with Gram positive bacteria.

Suitably the infection is an infection with Gram negative bacteria.

The peptides of the invention show surprising activity against Gram negative bacteria. Thus most suitably the infection is an infection with Gram negative bacteria.

The invention finds application in treatment of lung infection, suitably lung infection caused by any of the pathogens identified above.

The invention finds application in treatment of other indications where one or more of the bacterial species identified above may be found, for example applications might include:

i) skin/soft tissue,
ii) wound/burn,
iii) urinary tract infection (uncomplicated or complicated and including catheter-associated infections),
iv) sepsis; more suitably the peptide of the invention may be used in combination with other licensed antibiotics,
v) multidrug-resistant sexually transmitted disease, such as N. gonorrhoea,
vi) gastrointestinal infections such as those caused by C. difficile, Salmonella, spp, Shigella spp, Vibrio spp, Campylobacter spp, Helicobacter; more suitably those resistant to fluoroquinolones or other “standard of care” antibiotics (e.g. World Health Organisation (WHO) “High” Priority pathogens; a list is available from the WHO e.g. at http://www.who.int/medicines/publications/WHO-PPL-Short Summary 25Feb-ET NM WHO.pdf).

We demonstrate efficacy via intravenous injection in an in vivo model of pulmonary infection. This shows utility of the invention. The invention also finds application in AMP combinations for treating e.g. hospital acquired pneumonia/ventilator associated pneumonia (HAP/VAP).

Sequence Identity

For precision, sequence relationships have been discussed in terms of substitutions relative to SEQ ID NO: 1 (wild type Winter Flounder pleurocidin). However it may be desired to consider sequence relationships in terms of sequence identity.

Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example in an otherwise identical pair of sequences, one insertion or deletion will cause the following amino acid residues to be put out of alignment, thus potentially resulting in a large reduction in percent homology (percent identity) when a global alignment (an alignment across the whole sequence) is performed. Consequently, most sequence comparison methods are designed to produce optimal alignments that take into consideration possible insertions and deletions without penalising unduly the overall homology (identity) score. This is achieved by inserting “gaps” in the sequence alignment to try to maximise local homology/identity.

These more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package, FASTA (Altschul et al., 1990, J. Mol. Biol. 215:403-410) and the GENEWORKS suite of comparison tools.

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62. Once the software has produced an optimal alignment, it is possible to calculate percent homology, preferably percent sequence identity. The software typically does this as part of the sequence comparison and generates a numerical result.

### Reference Sequence

Suitably the current version of sequence database(s) are relied upon. Alternatively, the release in force at the date of filing is relied upon. For the avoidance of doubt, UniProt release 2019_10 is relied upon. In more detail, the UniProt consortium European Bioinformatics Institute (EBI), SIB Swiss Institute of Bioinformatics and Protein Information Resource (PIR)'s UniProt Knowledgebase (UniProtKB) Release 2019_10 published 13 Nov. 2019 is relied upon. UniProt (Universal Protein Resource) is a comprehensive catalogue of information on proteins (“UniProt: the universal protein knowledgebase” Nucleic Acids Res. 45: D158-D169 (2017)).

Pleurocidin is a known antimicrobial peptide from Winter Flounder and is sometimes referred to as “WF2” or “NRC-04”. The naturally occurring/wild type peptide is composed only of L-amino acids. Suitably all sequences herein are discussed with reference to pleurocidin from Winter Flounder. For the avoidance of doubt, this sequence is presented below:

SEQ ID NO: 1—“Pleurocidin” (25 amino acids):

### GWGSFFKKAAHVGKHVGKAALTHYL

When particular amino acid residues are referred to herein using numeric addresses, the numbering is taken with reference to the amino acid sequence SEQ ID NO: 1 (or to the polynucleotide sequence encoding same if referring to nucleic acid).

This is to be used as is well understood in the art to locate the residue of interest. This is not always a strict counting exercise—attention must be paid to the context. For example, if the protein of interest is of a slightly different length, then location of the correct residue in that sequence may require the sequences to be aligned and the equivalent or corresponding residue picked. This is well within the ambit of the skilled reader.

Mutating has its normal meaning in the art and may refer to the substitution or truncation or deletion of one or more residues, motifs or domains. Mutation may be effected at the polypeptide level, for example, by synthesis of a polypeptide having the mutated sequence, or may be effected at the nucleotide level, for example, by making a polynucleotide encoding the mutated sequence, which polynucleotide may be subsequently translated to produce the mutated polypeptide. Suitably, the mutations to be used are as set out herein. Unless otherwise apparent from the context, mutations mentioned herein are substitutions. For example ‘K7R’ means that the residue corresponding to ‘K7’ in the wild type pleurocidin sequence (SEQ ID NO: 1) is substituted with R.

SEQ ID NO: 2 - “Pleurocidin-KR”: substitutions
relative to SEQ ID NO: 1 bold and underlined.
(84% identical to SEQ ID NO: 1):
GWGSFFRRAAHVGRHVGRAALTHYL
SEQ ID NO: 3 - “Pleurocidin-VA”: substitutions
relative to SEQ ID NO: 1 bold and underlined.
(92% identical to SEQ ID NO: 1):
GWGSFFKKAAHAGKHAGKAALTHYL
SEQ ID NO: 4 - “Pleurocidin-KRVA”: substitutions
relative to SEQ ID NO: 1 bold and underlined.
(76% identical to SEQ ID NO: 1):
GWGSFFRRAAHAGRHAGRAALTHYL

The phrase ‘amino acid sequence derived from pleurocidin’ has its natural meaning. Suitably this phrase has its normal meaning in the art. Suitably an amino acid sequence ‘derived from’ a reference amino acid sequence means an amino acid sequence based on, or adapted from, the reference amino acid sequence. The amino acid sequence ‘derived from’ a reference amino acid sequence may comprise mutations relative to the reference sequence, for example substitutions. More suitably an amino acid sequence ‘derived from’ a reference amino acid sequence means an amino acid sequence having a sequence identity relationship with the reference amino acid sequence. Most suitably an amino acid sequence ‘derived from’ a reference amino acid sequence means an amino acid sequence having at least 52% sequence identity to the reference amino acid sequence, more suitably at least 56% sequence identity, more suitably at least 60% sequence identity, more suitably at least 64% sequence identity, more suitably at least 68% sequence identity, more suitably at least 72% sequence identity, more suitably at least 76% sequence identity, more suitably at least 80% sequence identity, more suitably at least 84% sequence identity, more suitably at least 88% sequence identity, more suitably at least 92% sequence identity, more suitably at least 96% sequence identity to the reference amino acid sequence.

Most suitably peptide of the invention comprises an amino acid sequence having at least 76% sequence identity, more suitably at least 80% sequence identity, more suitably at least 84% sequence identity, more suitably at least 88% sequence identity, more suitably at least 92% sequence identity, more suitably at least 96% sequence identity to the reference amino acid sequence.

As can be appreciated when the reference sequence is SEQ ID NO: 1 (pleurocidin), each 4% decrease in amino acid sequence identity corresponds to one substitution across the full length 25aa pleurocidin sequence. Thus, 96% sequence identity corresponds to one substitution across the full length 25aa pleurocidin sequence; 92% sequence identity corresponds to one substitution across the full length 25aa pleurocidin sequence; 88% sequence identity corresponds to one substitution across the full length 25aa pleurocidin sequence; and so on.

When the reference sequence is SEQ ID NO: 1 (pleurocidin), 52% amino acid sequence identity corresponds to 12 substitutions across the full length 25aa pleurocidin sequence. In one embodiment these 12 substitutions may be 4 K substitutions (suitably to R) and 2 V substitutions (suitably to A or L or I) and 3 His substitutions (suitably to 3-methylhistidine or 1-methylhistidine or 2,3-diaminopropionic acid or (N-methyl or N,N-dimethyl derivative) or glutamate or aspartate) and 2 phenylalanine substitutions (suitably to tyrosine or tryptophan) and one substitution of an amino acid from 9 to 21 relative to SEQ ID NO: 1 with a peptoid (suitably N-substituted glycine).

When the reference sequence is SEQ ID NO: 1 (pleurocidin), 76% amino acid sequence identity corresponds to 6 substitutions across the full length 25aa pleurocidin sequence. In one embodiment these 6 substitutions may be 4 K->R substitutions and 2 V->(A or L or I) substitutions, most suitably 4 K->R substitutions and 2 V->A substitutions.

In one embodiment if the amino acid sequence of interest is of a different length to the reference amino acid sequence, suitably sequence identity is calculated across the length of the amino acid sequence of interest. In this embodiment for example if the amino acid sequence of interest is 20 amino acids and the reference amino acid sequence is 25 amino acids then a sequence identity value of 100% is calculated if all 20 amino acids in the sequence of interest are identical to the corresponding amino acids in the reference sequence, which figure may be lower if there are substitutions within the 20 amino acids when aligned to the appropriate/corresponding 20 amino acids in the reference sequence—for example if there are 2 substitutions amongst the 20 amino acids relative to the reference sequence then a sequence identity value of 90% is calculated (2 amino acids substituted relative to the reference sequence=2 differences i.e. 18/20 amino acids identical=90% sequence identity).

Most suitably when the length of the peptide of interest is specified, sequence identity is calculated across the length of the peptide of interest.

In one embodiment if the amino acid sequence of interest is of a different length to the reference amino acid sequence, suitably sequence identity is calculated across the length of the reference amino acid sequence. In this embodiment any truncations (e.g. when the amino acid sequence of interest is shorter than the reference sequence) may be counted in the assessment of sequence identity so for example if the amino acid sequence of interest is 20 amino acids and the reference amino acid sequence is 25 amino acids then a sequence identity value of 80% is calculated, which figure may be lower if there are substitutions within the 20 amino acids when aligned to the appropriate/corresponding 20 amino acids in the reference sequence—for example if there are 2 substitutions amongst the 20 amino acids relative to the reference sequence then a sequence identity value of 72% is calculated (5 amino acids deleted relative to the reference sequence plus 2 amino acids substituted relative to the reference sequence=7 differences i.e. 18/25 amino acids identical=72% sequence identity). Most suitably this method of calculating sequence identity across the length of the reference sequence is used unless otherwise apparent from the context.

Most suitably sequence identity is calculated using conventional algorithms; these take account of differing lengths of the reference sequence and the sequence of interest using statistical adjustments (e.g. gap penalties etc), for example as explained above in the ‘sequence identity’ section.

It will be noted that in some embodiments, peptides of the invention may comprise unnatural amino acids or other residue(s) in place of one or more amino acid residue(s) in the reference sequence.

Thus amino acids in the peptide sequence may be substituted (relative to SEQ ID NO: 1) with “unnatural” amino acids i.e. amino acids not found in the 20 amino acids conventionally regarded as part of the universal genetic code. Any sequence identity calculation suitably takes account of these according to convention in the art.

For example, when a peptide contains one or more unnatural amino acids compared to the reference sequence, these are counted as substitution(s). For example when an alpha amino acid in the reference sequence is substituted for [N-substituted glycine] (i.e. peptoid) this is counted as a conventional substitution; and when histidine is substituted for 3-methylhistidine or 1-methylhistidine this is counted as a conventional substitution; when histidine is substituted for 2,3-diaminopropionic acid or an N-methyl or N,N-dimethyl derivative of 2,3-diaminopropionic acid, this is counted as a conventional substitution.

### Nomenclature

All the peptides are described herein using standard nomenclature unless otherwise indicated. For the avoidance of doubt, unless otherwise apparent, the peptides are suitably the C-terminal amidated forms. Peptide forms with a free acid C-terminus are also embraced with reference to the appended claims—these forms may be a little less potent but very similar in potency to the C-terminal amidated forms. Thus suitably the peptide of the invention is a free acid C-terminus peptide or a C-terminal amidated peptide. More suitably the peptide of the invention is a C-terminal amidated peptide, which has the advantage of slightly increased potency.

Sequences for pleurocidin, pleurocidin-VA (pleuro-VA) and pleurocidin-KR (pleuro-KR) are as presented herein.

The D-amino acid versions are the same sequences with all D-amino acids (e.g. D-pleuro, D-pleuro-VA, D-pleuro-KR).

D-pleuro-VAKR includes all preferred changes described herein (i.e. 6× substituted (4×K->R plus 2×V->A)) relative to the reference sequence. i.e. GWGSFFRRAAHAGRHAGRAALTHYL-NH2

Thus “Pleuro-D1” and “Pleuro-D2” are as follows:
modified Val16 from L- to D- in a first peptide (pleurocidin-D1) and also did this for both valines (Val12 and Val16) in a second peptide (pleurocidin-D2).

i.e.

Pleurocidin-D1

GWGSFFKKAAH [symbol] GKHVGKAALTHYL-NH2

and

Pleurocidin-D2

GWGSFFKKAAH [symbol] GKH [symbol] GKAALTHYL-NH2

where the boxed lower-case letters (e.g. “ [symbol] ”) indicate D-amino acids and the other amino acids are L-amino acids.

### Peptoids

Peptoids, or poly-N-substituted glycines, are a class of peptidomimetics whose side chains are appended to the nitrogen atom of the peptide backbone, rather than to the α-carbons (as they are in amino acids).

In particular it should be noted that peptoids lack the amide hydrogen which is responsible for many of the secondary structure elements in peptides and proteins. Peptoids were first invented by Simon et al 1992 (“Peptoids: a modular approach to drug discovery” Proceedings of the National Academy of Sciences USA, (1992), 89(20), 9367-9371). See also U.S. Pat. No. 5,811,387 (Simon et al 1998 “Peptoid Mixtures”, 22 Sep. 1998). They were originally used to mimic protein/peptide products to aid in the discovery of protease-stable small molecule drugs.

They are useful in the present invention as part of the peptide molecule in certain embodiments as explained herein.

One advantage of peptoids is resistance to proteolysis.

### Hinge Region

The ‘hinge region’ means amino acids 9 to 21 with reference to the sequence of pleurocidin (SEQ ID NO: 1)—the skilled reader can locate the corresponding hinge region on sequences derived from SEQ ID NO: 1 in the usual manner using the guidance provided herein e.g. by alignment allowing for possible sequence differences in the sequence of interest relative to SEQ ID NO: 1.

In one embodiment the peptide of the invention comprises addition of a peptoid backbone in the hinge region.

Suitably the phrase “addition of a peptoid backbone in the hinge region” means the substitution of N-substituted glycine into the molecule in place of an alpha amino acid. Such substitution(s) may be made anywhere within one turn of Gly13 or Gly17 i.e. at any one or more amino acids 9 to 21 relative to SEQ ID NO: 1. This has the technical effect of affecting the local conformation and hence manipulating the selectivity that the inventors attribute to the histidine residue(s) in this region.

In one embodiment suitably one substitution of N-substituted glycine in place of an alpha amino acid is made at one amino acid position selected from any of amino acids 9 to 21 relative to SEQ ID NO: 1.

The inventors assert that preparation of a peptide according to the present invention including a peptoid backbone in the hinge region is routine/well established for a person skilled in the art. The substitution(s) would suitably vary to match the character of the alpha amino acid(s) it/they would replace. In case any further guidance is needed, we refer to Kim et al 2010 (Biochimica et Biophysica Acta Volume 1798, Issue 10, October 2010, Pages 1913-1925 “Structural flexibility and the positive charges are the key factors in bacterial cell selectivity and membrane penetration of peptoid-substituted analog of Piscidin 1”.

### Structural Variations

Polypeptides include variants produced by introducing any type of additional alterations (for example, insertions, deletions, or substitutions of amino acids; changes in glycosylation states; changes that affect refolding or isomerizations, three-dimensional structures, or self-association states), which can be deliberately engineered. The variant may have alterations which produce a silent change and result in a functionally equivalent polypeptide. Deliberate amino acid substitutions may be made on the basis of similarity in polarity, charge, solubility, hydrophobicity, hydrophilicity and the amphipathic nature of the residues as long as the structure or conformation of the polypeptide is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the table below. Amino acids in the same block in the second column and suitably in the same line in the third column may be substituted for each other:

| | | |
|---|---|---|
| ALIPHATIC | Non-polar | GAP |
| | | ILV |
| | Polar—uncharged | CSTM |
| | | NQ |
| | Polar—charged | DE |
| | | KR |
| AROMATIC | | HFWY |

In considering what mutations, substitutions or other such changes might be made relative to the wild type sequence, retention of the structure or conformation of the polypeptide is important. Typically conservative amino acid substitutions would be less likely to adversely affect the function.

Thus in case any further guidance is required, the inventors teach that replacing like for like amino acids is most suitable. The inventors teach to pay attention to the outcome, i.e. does the modified peptide achieve the same properties i.e. increased conformational flexibility, membrane damage and/or selectivity. The inventors teach that some “conservative” changes might not be that subtle but if their effect is similar to that of the changes we have described then it would be suitable. The inventors teach that substitutions should maintain activity. The inventors teach that like for like changes at sites distant to specific residues of interest are likely to not make a difference to activity whereas like for like changes at site of interest are likely to maintain functional activity. More suitably, regarding ‘distant to’, like for like changes more than 4 amino acids distant in the primary sequence from sites of specific residues of interest means that functional activity is likely unaffected and preserved. Changes at site of interest that are dissimilar to amino acids as stipulated will be disruptive and therefore should be avoided or excluded.

### Combinations of Substitutions

Suitably the peptide of the invention contains one or more amino acid substitution(s) relative to the histidine(s) occurring in the pleurocidin sequence (SEQ ID NO: 1).

Suitably the peptide of the inventions contains one or more substitution(s) relative to the tryptophan/glycine residue(s) in the parent pleurocidin sequence (SEQ ID NO: 1).

Suitably, the peptide of the invention contains one or more substitution(s) relative to the occurrence of phenylalanine in the parent pleurocidin sequence (SEQ ID NO: 1).

Suitably the peptide of the invention contains one or more substitution(s) relative to the occurrence of lysine in the parent pleurocidin sequence (SEQ ID NO: 1).

Suitably the peptide of the invention contains one or more substitution(s) relative to the occurrence of valine in the parent pleurocidin sequence (SEQ ID NO: 1).

Even more suitably, the peptide of the invention contains a substitution relative to the lysine amino acid(s) occurring in the pleurocidin sequence and contains a substitution relative to the valine amino acid(s) occurring in the pleurocidin parent peptide sequence.

The particular substitutions disclosed herein relative to SEQ ID NO: 1 may be combined whether or not each combination is specifically shown as an example herein.

Thus in one embodiment the invention relates to a peptide having the following formula (SEQ ID NO: 24):

### GWGSFX1X2X3X4AAX5X6GX7X8X9GX10AALTX11YL

wherein X1 is F or W or Y;
wherein X2 is F or W or Y;
wherein X3 is K or R;
wherein X4 is K or R;
wherein X5 is H or 3-methylhistidine or 1-methylhistidine or 2,3-diaminopropionic acid or (N-methyl or N,N-dimethyl derivative of 2,3-diaminopropionic acid) or E or D;
wherein X6 is V or A or L or I;
wherein X7 is K or R;
wherein X8 is H or 3-methylhistidine or 1-methylhistidine or 2,3-diaminopropionic acid or (N-methyl or N,N-dimethyl derivative of 2,3-diaminopropionic acid) or E or D;
wherein X9 is V or A or L or I;
wherein X10 is K or R;
wherein X11 is H or 3-methylhistidine or 1-methylhistidine or 2,3-diaminopropionic acid or (N-methyl or N,N-dimethyl derivative of 2,3-diaminopropionic acid) or E or D;
wherein one or more of amino acids 9 to 21 is optionally substituted for N-substituted glycine; and
wherein if X1 is F, and X2 is F, and X3 is K, and X4 is K, and X5 is H, and X6 is V, and X7 is K, and X8 is H, and X9 is V, and X10 is K, and X11 is H, and none of amino acids 9 to 21 is optionally substituted for N-substituted glycine, then at least one of the amino acids is a D-amino acid.

In another embodiment the invention relates to a peptide comprising amino acid sequence having at least 52% sequence identity to SEQ ID NO: 1,
wherein said amino acid sequence comprises one or more of:
a) Substitution of one to four lysine(s) with arginine;
b) Substitution of one or two valine(s) with a different amino acid, suitably said one or two valine(s) being substituted with an amino acid selected from the group consisting of alanine, leucine and isoleucine;
c) Substitution of one or more phenylalanine(s) with tyrosine or tryptophan;
d) Substitution of one or more of amino acids 9 to 21 relative to SEQ ID NO: 1 with N-substituted glycine;
e) Substitution of one or more histidine(s) with 3-methylhistidine or 1-methylhistidine;
f) Substitution of one or more histidine(s) with 2,3-diaminopropionic acid or N-methyl or N,N-dimethyl derivatives thereof; or
g) Substitution of one or more histidine(s) with glutamate or aspartate, relative to SEQ ID NO: 1.

Suitably said peptide comprises (a) Substitution of one to four lysine(s) with arginine, relative to SEQ ID NO: 1.

Suitably said peptide comprises (b) Substitution of one or two valine(s) with a different amino acid, suitably with alanine, relative to SEQ ID NO: 1.

Suitably said peptide comprises two or more of (a) to (g) relative to SEQ ID NO: 1.

Suitably said peptide comprises (a) Substitution of one to four lysine(s) with arginine and (b) Substitution of one or two valine(s) with a different amino acid, suitably with alanine, relative to SEQ ID NO: 1.

Suitably said peptide comprises substitution of four lysines with arginine, relative to SEQ ID NO: 1.

Suitably said peptide comprises substitution of two valines with alanine, relative to SEQ ID NO: 1.

Suitably said peptide comprises substitution of four lysines with arginine and substitution of two valines with alanine, relative to SEQ ID NO: 1.

In another embodiment the invention relates to a peptide comprising amino acid sequence having at least 52% sequence identity to SEQ ID NO: 1, wherein said amino acid sequence comprises one or more of the following substitutions relative to SEQ ID NO: 1:
a) F5Y or F5W
b) F6Y or F6W
c) K7R
d) K8R
e) substitution of H11 for 3-methylhistidine or 1-methylhistidine or 2,3-diaminopropionic acid or (N-methyl or N,N-dimethyl derivative of 2,3-diaminopropionic acid) or E or D;
f) V12A or V12I or V12L
g) K14R
h) substitution of H15 for 3-methylhistidine or 1-methylhistidine or 2,3-diaminopropionic acid or (N-methyl or N,N-dimethyl derivative of 2,3-diaminopropionic acid) or E or D
i) V16A or V16I or V16 L
j) K18R
k) substitution of H23 for 3-methylhistidine or 1-methylhistidine or 2,3-diaminopropionic acid or (N-methyl or N,N-dimethyl derivative of 2,3-diaminopropionic acid) or E or D; or
l) substitution of one or more of amino acids 9 to 21 for N-substituted glycine.

In another embodiment the invention relates to a peptide comprising amino acid sequence having at least 52% sequence identity to SEQ ID NO: 1, wherein said amino acid sequence comprises one or more of the following substitutions relative to SEQ ID NO: 1:
a) F5Y or F5W
b) F6Y or F6W
c) K7R
d) K8R
e) substitution of H11 for 3-methylhistidine or 1-methylhistidine or 2,3-diaminopropionic acid or (N-methyl or N,N-dimethyl derivative of 2,3-diaminopropionic acid) or E or D;
f) V12A or V12I or V12L
g) K14R
h) substitution of H15 for 3-methylhistidine or 1-methylhistidine or 2,3-diaminopropionic acid or (N-methyl or N,N-dimethyl derivative of 2,3-diaminopropionic acid) or E or D
i) V16A or V16I or V16 L
j) K18R
k) substitution of H23 for 3-methylhistidine or 1-methylhistidine or 2,3-diaminopropionic acid or (N-methyl or N,N-dimethyl derivative of 2,3-diaminopropionic acid) or E or D.

In one embodiment suitably said peptide comprises at least the following substitutions relative to SEQ ID NO: 1:
c) K7R
d) K8R
g) K14R
j) K18R.

In one embodiment suitably said peptide comprises at least the following substitutions relative to SEQ ID NO: 1:
f) V12A or V12I or V12L
i) V16A or V16I or V16 L.

In one embodiment suitably said peptide comprises at least the following substitutions relative to SEQ ID NO:1:
c) K7R
d) K8R
f) V12A or V12I or V12L
g) K14R
i) V16A or V16I or V16 L
j) K18R.

In one embodiment, the peptide of the invention contains both one or more lysine to arginine substitution(s) and one or more valine to alanine, isoleucine or leucine substitution(s) relative to the pleurocidin sequence (SEQ ID NO: 1). In one embodiment, the peptide of the invention contains both one or more lysine to arginine substitution(s) and one or more valine to alanine substitution(s) relative to the pleurocidin sequence (SEQ ID NO: 1).

### Manufacture

Peptides described herein may be made by any suitable technique known to the skilled worker. For example peptides may be prepared by conventional synthesis. Numerous service companies can easily manufacture such peptides, for example Cambridge Research Biochemicals (Cleveland, UK).

All peptides would normally be assembled using solid-phase synthesis. This includes peptides comprising (for example) MethylHistidine or DAP or other substitutions. The manufacture of the peptides/modifications disclosed herein is not ‘special’—the person skilled in the art can prepare the polypeptides disclosed herein. For example, routes to incorporate N-methyl-2,3-diaminopropionic acid (and N,N-dimethyl-2,3-diaminopropionic acid) in peptides are known in the art. All variants can therefore be produced using standard techniques.

### Testing

The skilled worker can select appropriate tests to assess the characteristics of peptides described herein. For the avoidance of doubt, guidance is provided herein in particular in the examples section.

It is noted that Mueller Hinton (Mueller Hinton Broth (MHB)) tests and RPMI (Roswell Park Memorial Institute 1640 (RPMI)) tests are different. The person skilled in the art will take this into account when interpreting the results. Optimally, both tests are carried out when studying peptides such as those disclosed herein. For example, the synergy with serum is revealed particularly clearly/strongly when testing using RPMI. Thus, RPMI is a very valuable test when studying differences in potency which can be dependent on the bacterial species which the peptide is tested against.

### D-Amino Acids and L-Amino Acids

Suitably the amino acids used to produce the peptides of the invention are D-amino acids. Use of D-amino acids may provide a more potent peptide. Without wishing to be bound by theory, it is believed that D-amino acids make the peptide more stable.

Thus in one embodiment the peptides of the invention comprise D-amino acids.

Thus, in one embodiment, the peptides of the invention may be composed of D-amino acids.

In one embodiment the peptides of the invention comprise only D-amino acids.

In one embodiment the peptides of the invention comprise all D-amino acids.

In one embodiment the peptides of the invention consist of D-amino acids.

In one embodiment each of the amino acids in the peptide of the invention is a D-amino acid.

It is believed that L-amino acids are more easily biodegraded. This can provide an advantage when it is desired for the peptide of the invention to be more rapidly biodegraded. In one embodiment, suitably the peptide of the invention contains lysine to arginine substitutions relative to pleurocidin sequence.

Thus, in one embodiment, the peptides of the invention comprise L-amino acids.

Thus, in one embodiment, the peptides of the invention may be composed of L-amino acids.

In one embodiment the peptides of the invention comprise only L-amino acids.

In one embodiment the peptides of the invention comprise all L-amino acids.

In one embodiment the peptides of the invention consist of L-amino acids.

In one embodiment each of the amino acids in the peptide of the invention is an L-amino acid.

Whether particular amino acids are D- or L-amino acids is apparent from the context where different embodiments are discussed. For the avoidance of doubt, substitutions are discussed in the conventional manner so for example incorporating a D-valine in place of an L-valine relative to the amino acid sequence of SEQ ID NO: 1 is not counted as a substitution; the presence of D-amino acids and/or L-amino acids in the sequence of interest does not affect the assessment of sequence identity as described herein.

Although the wild type amino acid sequence of Pleurocidin is known (SEQ ID NO: 1—the most suitable reference sequence as explained herein), this wild type sequence is comprised of L-amino acids. Therefore the disclosure herein to produce D-pleurocidin i.e. a peptide comprising, or consisting of, the amino acid sequence of SEQ ID NO: 1, comprising, or consisting of, D-amino acids is itself a novel peptide disclosed herein. Thus in one aspect is disclosed a peptide comprising, or consisting of, the amino acid sequence of SEQ ID NO: 1, wherein said peptide comprises, or consists of, D-amino acids. In one embodiment suitably said peptide is composed entirely of D-amino acids.

In one embodiment suitably said peptide does not comprise any L-amino acids.

### Mixed L-Amino Acid(s) and D-Amino Acid(s) Peptides

In a minor embodiment the peptides of the invention may comprise both L-amino acid(s) and D-amino acid(s). Peptides Pleuro-D1 and Pleuro-D2 are examples of peptides comprising both D- and L-amino acids. These are sometimes referred to as ‘mixed L-amino acid/D-amino acid’ peptides (i.e. a mix of L and D amino acids in the same peptide). These are not preferred. Preferred are ‘all-D’ or ‘all-L’ amino acid peptides as explained above. Without wishing to be bound by theory, the inventors believe that the single and double L- to D-amino acid replacements may impair function. Without wishing to be bound by theory, data indicates that introducing only one or two D-amino acids into an L-amino acid background may reduce activity. We refer to Example 5. Thus suitably the peptides Pleuro-D1 and Pleuro-D2 are disclaimed. Suitably mixed peptides (i.e. ‘mixed L-amino acid/D-amino acid’ peptides) are not part of the invention.

### Lysine Substitution

The inventors have shown the importance of advantages which can be gained from substituting one or more lysines in sequences derived from the reference sequence (pleurocidin sequence—SEQ ID NO: 1) to arginine. It should be noted that both lysine and arginine are basic amino acids. It should be noted that both lysine and arginine have the same pKa values. However, an important insight brought by the inventors is that lysine residues and arginine residues differ in their capability for hydrogen bonding. It is a key part of the teaching brought by the inventors that the balance between alpha helix formation and membrane interaction is attributable to the hydrogen bond balance in the molecule. Therefore the inventors have used their insights to make intellectual decisions devising alternate peptide structures through substitutions with both natural and unnatural amino acids so as to achieve novel technical benefits such as improved membrane interaction and/or decreased stability of the alpha helical structure. It should be noted that this approach goes against what has been attempted in the prior art, because prior art approaches prefer to enhance or maintain the stability of alpha helical structures. Loss of stability of the alpha helical structure is considered a bad or detrimental effect in the prior art approaches. Therefore the invention teaches directly against thinking in the art.

In the prior art, nobody would be motivated to make a lysine to arginine substitution in order to change the secondary structure of a polypeptide.

It is possible that the lysine to arginine substituted peptide is more toxic than the naturally occurring peptide. Table 2 may demonstrate this enhanced toxicity. However, in some embodiments the lysine to arginine substituted peptides of the invention are still more suitable for use because despite their higher toxicity they can still provide a higher therapeutic index. Thus, it may be possible to use a lower quantity of the more toxic peptide and still achieve a more beneficial therapeutic effect.

It should be noted that the inventors teach lysine to arginine substitutions. The view in the art is that this would retain the alpha helical structure. The view in the art is that disrupting the alpha helical structure makes a peptide less active (e.g. see Cuervo et al 1988 (ibid.)). By contrast, the present invention goes against this prejudice in the art. This is further evidence towards the inventiveness/non-obviousness of the present invention.

When making a substitution relative to lysine occurring in the parent pleurocidin sequence, suitably lysine is substituted for arginine. In another embodiment, suitably lysine is substituted for citrulline. Citrulline has the same hydrogen bonding capability as lysine but does not have the charge which lysine has.

### Valine Substitution

In another embodiment, suitably the peptide contains a valine substitution, more suitably a valine to alanine, isoleucine or leucine substitution, more suitably valine to alanine substitution, relative to the pleurocidin sequence, or contains a plurality of (such as two) valine substitutions, more suitably a plurality of (such as two) valine to alanine, isoleucine or leucine substitutions, more suitably a plurality of (such as two) valine to alanine substitutions relative to the pleurocidin peptide. An advantage of peptides with valine substitution(s) relative to pleurocidin is that a greater maximum tolerated dose may be used in treatment.

A further advantage of valine substitution(s) in the peptides of the invention is an increased protease resistance. Thus, it was further surprising to observe an increased potency for peptides of the invention comprising valine substitution(s), since any motivation for making such substitutions might be in considering protease resistance rather than potency. Furthermore, a valine substitution such as a valine to alanine (or isoleucine or leucine) substitution is expected to be a conservative substitution in the context of an alpha helical peptide. Therefore, it is very surprising that a valine to alanine (or isoleucine or leucine) substitution in an alpha helical part of the protein would lead to conformational flexibility as demonstrated herein.

In the art a valine to alanine (or isoleucine or leucine) substitution is considered conservative. In the art, workers try to increase the positive charge on a peptide and/or try to increase the hydrophobicity of a peptide in order to try to achieve greater interaction with a membrane. Indeed, considering pleurocidin, this parent peptide is more hydrophilic than most candidate AMPs. Therefore, there is an even greater reason without an understanding of the invention to try to make it more hydrophobic according to principles derived from the prior art. In contrast, the inventors' approach, including valine to alanine (or isoleucine or leucine) substitutions, has gone against thinking in the art and is therefore supportive of inventive step/non-obviousness.

### Histidine Substitution

Suitably the peptide of the invention contains one or more substitution(s) of histidine residue(s) relative to the parent pleurocidin sequence (SEQ ID NO: 1). For example, protonation of a histidine residue can be dependent on the state of the local environment. For example, the orientation of the histidine residue can be important in the context of the peptide. Thus, for at least these reasons, the inventors teach that histidine may be substituted for methylhistidine; the inventors teach that the histidine may be substituted for diaminopropionic acid (DAP).

Substituting the naturally occurring histidines derived from the reference sequence (pleurocidin sequence—SEQ ID NO: 1) has been shown to be very important for selectivity. Changing one or more of the naturally occurring histidine(s) for other amino acid residues or other moieties (such as non-natural moieties e.g. DAP) provides the advantageous property of maximising membrane interaction.

### Subject

Suitably the subject is a human or a non-human animal.

In one embodiment suitably the subject is a small animal.

In one embodiment suitably the subject is a large animal such as a pig.

Suitably the subject is a mammal.

Most suitably the subject is a human.

### Dose/Administration

In one embodiment delivery/administration may be topical delivery. This may be especially suitable for delivery to skin and/or soft tissue infections or wounds.

Most suitably delivery/administration is systemic.

Suitably delivery/administration may be oral, i.v. or inhaled.

Administration may be by delivery to the lung.

Administration may be by delivery to the gastrointestinal (GI) tract.

Administration may be by delivery to the skin or soft tissue.

Administration may be by nebulisation, or in a dry powder for example using a metered dose inhaler.

More suitably administration is by injection, most suitably i.v. injection.

More suitably the peptide of the invention is administered to a subject intravenously (i.v.) or intramuscularly (i.m.).

Most suitably the peptide of the invention is administered to a subject intravenously (i.v.).

Formulations/delivery options may include one or more of the following:

i) formulation into a inhaled or nebulised powder or liposome formulation for delivery to the lung;
ii) formulation into a gel/wound dressing for topical delivery to skin and soft tissue infections or wounds,
iii) production of a tablet or capsule suitable for treatment of the GI tract.

The invention finds application in the clinical market i.e. human use. The invention further finds application for use in non-human animals such as small animals. Byway of example, known treatment PEPTIVET® is indicated for treatment of Pseudomonas aeruginosa causing canine otitis and contains the antimicrobial peptide AMP2041 as well as chlorhexidine and EDTA. Thus suitably the peptide of the invention may be co-administered with chlorhexidine and EDTA, most suitably co-administered with chlorhexidine and EDTA for veterinary use.

The invention finds application in use as a single treatment, or in combination with other antimicrobials (such as other AMPs) for example to treat lung infections in mammals such as larger mammals, for example pigs where chronic respiratory disease is the most economically important disease that affects growing and finishing pigs.

Suitably the peptide of the invention is administered to a subject with one or more doses delivered daily.

Suitably the peptide of the invention is administered to a subject with one dose delivered daily.

For humans, the dose is suitably 0.25-0.5 mg/Kg, for example for adult humans an exemplary dose is 0.25-0.5 mg/kg every 8-12 hours administered intravenously (i.v.). Suitably such a dose is for treatment of infection such as treatment of bacterial infection.

In case any further guidance is needed, by comparison with vancomycin, peptides of the invention show approx. 70 fold difference in potency (vancomycin effective at 200 mg/kg cumulative dose over 48 hrs, D-pleurocidin-KR effective at 1.5 mg/kg over 48 hrs). Suitably peptide of the invention may be used at 3 mg/kg for greater surety. Thus an exemplary dose of peptide according to the invention (such as D-pleurocidin-KR) may comprise i.v. dosing in humans at 0.25-0.5 mg/kg every 8-12 hours.

It is an advantage of the invention that the peptides are very water soluble. Thus suitably the formulation is an aqueous formulation.

For injection, the peptides of the invention are formulated in suitable buffer such as saline.

Suitably saline may be any suitable formulation for administration to a mammalian subject, suitably a human subject. Suitably saline comprises 0.9% NaCl. Suitably saline is as available from Baxter Healthcare Catalogue Number UKF7124 (Baxter Healthcare, Wallingford Road, Compton, Newbury, Berkshire, RG20 7QW, U.K.).

When PBS saline (Phosphate Buffered Saline) is used, suitably this is as available from Oxoid/ThermoScientific Catalogue Number BR0014G (Oxoid Limited, Wade Road, Basingstoke, Hampshire, RG24 8PW, United Kingdom).

For example, PBS may have the formula:

| Formula | gm/litre |
|---|---|
| Sodium chloride | 8.0 |
| Potassium chloride | 0.2 |
| Disodium hydrogen phosphate | 1.15 |
| Potassium dihydrogen phosphate | 0.2 |
| pH 7-3 | |

Most suitably PBS has the composition:

| | |
|---|---|
| Sodium chloride | 0.137 mol |
| Potassium chloride | 0.003 mol |
| Disodium hydrogen phosphate | 0.008 mol |
| Potassium dihydrogen phosphate | 0.0015 mol |

For pulmonary delivery suitably the peptide is delivered in a dry powder formulation. In case any further guidance is needed, by comparison with tobramycin, the dose of peptide according to the present invention may be similar—thus suitably the peptide of the invention is administered in the range 50-200 mg every 12 hours for 28 days for pulmonary delivery Suitably dry powder formulations of peptides are prepared as previously described (Kwok et al International Journal of Pharmaceutics vol 491 (2015) pages 367-374). Suitably the peptide of the invention is formulated using mannitol as excipient.

The skilled worker or physician will take account of the route/formulation/dose to provide suitable PK/PD profiles so as to ensure effective treatment of the infection in the relevant body compartment and/or bodily fluid (e.g. urine, wound exudate).

### Combinations of Antimicrobial Agents

When used in combinations with antibiotic agents the inventors show significantly reduced minimum inhibitory concentrations (MICs) against target species and the inventors assert that this provides the benefit of minimising the potential for resistance emergence.

Peptides of the invention (such as DPKR or DPVA) may be used in a combination therapy with other antibiotic agents such as colistin or tobramycin. This has the advantage(s) of allowing lower doses of each compound to be used, and/or overcoming infections caused by bacteria with existing resistance mechanisms (e.g. colistin), and/or to reduce treatment time to achieve clearance.

Two (or more) of the peptides of the invention may be used in combination, which has the advantage(s) of allowing lower doses of each compound to be used, and/or overcoming infections caused by bacteria with existing resistance mechanisms (e.g. colistin), and/or minimising the potential for resistance emergence and/or to reduce treatment time to achieve clearance.

### Further Embodiments

In one aspect the invention relates to use of an antimicrobial peptide as an antibacterial agent.

In one aspect the invention relates to use of an antimicrobial peptide to treat bacterial infection.

In one aspect the invention relates to use of an antimicrobial peptide as a systemic antibacterial agent.

In one aspect the invention relates to systemic administration of an antimicrobial peptide.

In one aspect the invention relates to a method of treating bacterial infection, the method comprising administration of an antimicrobial peptide. Suitably said administration is systemic administration.

In one embodiment suitably the bacterial infection is a lung infection.

The invention is described by way of numbered paragraphs:
Paragraph 1—a peptide comprising amino acid sequence derived from pleurocidin, comprising one or more of:
   a. one to four lysine to arginine substitutions;
   b. Substitution of one or more phenylalanine(s) with tyrosine or tryptophan residues;
   c. Substitution of one valine (either Val12 or Val16) with another amino acid, suitably with alanine;
   d. addition of a peptoid backbone in the hinge region;
   e. Substitution of one or more histidine residue(s) with 3-methylhistidine or 1-methylhistidine;
   f. Substitution of one or more histidine residue(s) with 2,3-diaminopropionic acid or N-methyl or N,N-dimethyl derivatives thereof; or
   g. Substitution of one or more histidine residue(s) with glutamate or aspartate.

Paragraph 2. A peptide according to paragraph 1, comprising two or more of (a) to (g).

Paragraph 3. A peptide according to paragraph 1 or paragraph 2 for use in medicine.

Paragraph 4. A peptide according to paragraph 1 or paragraph 2 or paragraph 3 for use in treatment or prevention of infection, suitably bacterial infection.

### Advantages of the Invention

MOA of hydrophobic AMPs can be associated with specificity.
Adjustment of the amount and positioning of lysine and aromatic residues changes MOA, potency and spectrum of activity.
Utilization of combined experimental and computational approaches is effective in the design of AMPs and selection of promising drug candidates.

It is an advantage of the invention that an enhanced membrane disruptive effect is produced in the peptides of the invention. By “enhanced” it is meant in a comparison to the naturally occurring pleurocidin peptide (SEQ ID NO: 1), the peptides of the invention show a higher capacity for membrane disruption than the pleurocidin peptide. In particular, this technical benefit is demonstrated by FIG. 3 (especially FIG. 3 B/F/J). This shows the effect demonstrated on lipid bilayers. It should be noted by the skilled reader that action on lipid bilayers is demonstrating a different mechanism of action to the parental pleurocidin peptide. Thus, the inventors have gone further than merely improving an existing molecule such as pleurocidin, they have in fact engineered new biochemical properties into the new molecules which are taught herein.

In addition, the inventors refer to FIG. 3 which demonstrates an unexpected transformation of behaviour when comparing peptides of the invention to prior art peptides. See also conductance data herein.

It was a surprise to the inventors to achieve a transformation of behaviour of the peptide following the lysine to arginine substitutions. This type of transformation of behaviour is not expected—this behaviour is demonstrated in FIG. 3.

Thus in some embodiments, it may be that the lysine to arginine substituted peptide may be preferred to the valine to alanine substituted peptide, for example when it is desired to achieve the transformation of behaviour as noted above.

It is an advantage of the invention that a greater potency is provided. In this embodiment, a greater potency refers to a greater membrane disruptive effect such as a greater bacterial membrane disruptive effect; greater means greater by comparison to the pleurocidin reference peptide (SEQ ID NO: 1). This advantageous effect would not be anticipated from an understanding of the prior art.

It is an advantage of the invention that the peptides taught herein are more potent against Gram-negative bacteria. ‘More potent’ means by comparison to the pleurocidin reference peptide (SEQ ID NO: 1). An expectation from the art is that peptides are usually more potent against Gram-positive bacteria. Therefore, the peptides taught herein having more potent activity against Gram-negative bacteria is itself surprising.

Further particular and preferred aspects are set out in the accompanying independent and dependent claims. Features of the dependent claims may be combined with features of the independent claims as appropriate, and in combinations other than those explicitly set out in the claims.

Here is described a Pleurocidin Analogue with Greater Conformational Flexibility, Enhanced Antimicrobial Potency and in vivo Efficacy.

KEYWORDS peptide antibiotics, MD simulations, pleurocidin, EMRSA-15, Pseudomonas aeruginosa RP73, systemic delivery, lung infection.

The inventors carried out rational design of the pleurocidin peptide to improve its antibacterial properties (MIC), reduce the protease/serum liability and extend the spectrum of activity.

In an exemplary embodiment substituting lysine amino acids to arginine within the pleurocidin backbone together with changing all L-amino acids to their D-amino acid counterparts (peptide DPKR—SEQ ID NO: 2 consisting of D-amino acids) gave rise to unexpected benefits. These include much improved activity against Gram-positive pathogens, which were not observed to the same extent in the L-amino acid version. The DPKR embodiment also showed significantly better activity in serum, when compared to either pleurocidin (SEQ ID NO: 1, L-amino acids), D-pleurocidin (SEQ ID NO: 1, D-amino acids) or the pleurocidin KR variant (SEQ ID NO: 2 consisting of L-amino acids). This was particularly evident for P.aeruginosa where the pleurocidin KR variant was observed to have markedly reduced activity in the presence of serum compared to the exceptional activity of DPKR. Again this is evidence of an unexpected technical benefit which was not predictable from the art but has significant clinical importance given that treatment of P.aeruginosa is a key multi-drug-resistant (MDR) target species with few viable treatment options.

Another exemplary embodiment substituting valine amino acids to alanine also gave unexpected effects in both the pleurocidin (PVA i.e. SEQ ID NO: 3, L-amino acids) and D-pleurocidin (DPVA i.e. SEQ ID NO: 3, D-amino acids) embodiments, with a significant reduction in the cellular toxicity. This significantly increases the therapeutic window for treatment of some of the Gram-negative species, notably A. baumannii, from around 40-80 fold MIC to >200 fold MIC. Again this is evidence of an unexpected technical benefit of the peptides of the invention.

Also shown is strong synergy between the peptides of the invention and both colistin and tobramycin. Notably, the DPKR peptide shows synergy with both colistin and tobramycin in P.aeruginosa and also with colistin in A.baumannii. Given the expected mechanism of action of the peptides of the invention, with at least part of their activity expected to be mediated by membrane disruption, this is a very beneficial effect. This is a further unexpected benefit of the exemplary peptides such as DPKR and DPVA.

Another key technical advantage of the peptides of the invention over known AMPs is the ability to use them (e.g. DPKR) as systemically delivered antibiotics. This broadens their clinical utility significantly compared to known approaches.

The invention is further described by examples which are intended to be illustrative and are not intended to limit the scope of the invention as defined by the appended claims.

### Examples

SUMMARY: Antimicrobial peptides (AMPs) are a potential alternative to classical antibiotics that are yet to achieve a therapeutic breakthrough for treatment of systemic infections. The antibacterial potency of pleurocidin, an AMP from Winter Flounder, is linked to its ability to cross bacterial plasma membranes and seek intracellular targets while also causing membrane damage. Here we describe modification strategies that generate pleurocidin analogues with substantially improved, broad spectrum, antibacterial properties, which are effective in murine models of bacterial lung infection when delivered intravenously (i.v.). Increasing peptide-lipid intermolecular hydrogen bonding capabilities enhances conformational flexibility but also membrane damage and potency, most notably against Gram-positive bacteria, and reduces sensitivity to the bacterial metabolic strategy. An analogue comprising D-amino acids was well tolerated at an i.v. dose of 15 mg/kg and similarly effective as 600 mg/kg vancomycin in reducing EMRSA-15 lung CFU. This highlights the therapeutic potential of systemically delivered, bactericidal AMPs.

### Introduction

The 2016 Review on Antimicrobial Resistance¹ (AMR) predicts that, unless action is taken, around 10 million deaths per year will be attributable to AMR by the year 2050. Action recommended by the review is two-fold: 1) that the inappropriate use of existing antimicrobials should be reduced so that their utility endures for longer and; 2) new antimicrobials must be made available that are effective against drug-resistant bacteria. The pipeline of new antibiotics is limited however, hence the potential of numerous alternatives to antibiotics—“noncompound approaches (i.e. products other than classic antibacterial agents) that target bacteria or approaches that target the host”—is being actively investigated.² Commissioned by the Wellcome Trust, a pipeline portfolio review of alternatives to antibiotics recommends “strong support for funding while monitoring for breakthrough insights regarding systemic therapy” for a tier of approaches that include AMPs.² The review presents the prevailing wisdom that AMPs are unsuited for systemic administration as they are poorly tolerated in animal models and susceptible to degradation. This substantially limits the scope of infection settings that are tractable to AMPs and hence their future development. There is an urgent need therefore to identify AMPs that are sufficiently potent against antibiotic resistant bacteria and well tolerated in vivo such that they are effective when delivered intravenously.

AMPs are a well-studied subset of a group of peptides that contribute to innate immunity, in a diverse range of organisms, through direct antimicrobial action and/or host defence regulation.^{3,4} Identified in the Winter Flounder⁵, Pleuronectes americanus, pleurocidin is a potent AMP with broad spectrum anti-bacterial activity that seemingly belongs in the class of AMPs that acts by damaging the plasma membrane,⁶ with activity that is dependent on their ability to adopt an amphipathic α-helix conformation.⁷ It is now well established however that many AMPs can disrupt bacterial cell metabolism, in addition to, or in place of, their well-known membrane damaging action.⁸ Pleurocidin is one such AMP and previous work supports the view that its high potency, at least against Gram-negative bacteria such as Escherichia coli, is linked to its ability to cross the bacterial plasma membrane and penetrate within bacteria to attack intracellular targets.^{9,10} Importantly, the ordered α-helix conformation that pleurocidin adopts in many membrane mimics or models is less apparent in those models that most closely represent a Gram-negative bacterial cytoplasmic membrane.^{11,12} The increased conformational flexibility, detected when pleurocidin binds to such membranes, affords greater ability to penetrate into the hydrophobic core of the lipid bilayer,¹² a property that we infer is critical to the potency associated with its intracellular penetration.

Strategies that increase the conformational flexibility of pleurocidin, when binding to model membranes, are likely to substantially alter its biological properties. Here we report the results of two such strategies. First, we hypothesized that substituting less bulky and less hydrophobic alanines for valines (both Val12 and Val16; pleurocidin-VA Table 1) located near to two key glycine residues (respectively Gly13 and Gly17) would have substantial impact on conformational flexibility in pleurocidin.^{12,13} Secondly, we hypothesized that substituting arginine for each of the four lysine residues (Lys7, Lys8, Lys14, Lys18; pleurocidin-KR Table 1) would directly increase hydrogen bonding between the peptide and the lipid headgroups, shifting the balance away from intramolecular hydrogen bonding that stabilizes ordered α-helix conformation.

Having solved the structures of the new analogues, we used a combination of both time-resolved and steady-state biophysical methods to determine the impact of the modifications on the membrane interaction to determine whether the modifications enhance and/or alter the peptide properties and bactericidal mechanisms of action. We evaluated the in vitro toxicity against mammalian cells and antibacterial performance against a panel of both Gram-negative and Gram-positive bacterial pathogens for the three pleurocidin analogues, and their all D-amino acid enantiomers, in both bacteriological and mammalian cell culture media. Further, we used an NMR metabolomic approach to investigate: 1) whether epidemic methicillin-resistant Staphylococcus aureus (EMRSA-15) or Pseudomonas aeruginosa RP73 distinguish between the (D-enantiomer) analogues to understand whether altered membrane interaction properties are manifested when challenging pathogens in vitro and; 2) whether altering the metabolic strategy of the bacteria renders them more or less susceptible to D-pleurocidin or its analogues. Finally, the therapeutic ability of D-pleurocidin-KR was demonstrated in murine models of EMRSA-15 and, in combination with tobramycin, P. aeruginosa RP73 lung infection, when delivered intravenously.

### Results

Peptide-lipid hydrogen bonding is directly and indirectly altered in pleurocidin analogues—In designing both in silico and in vitro experiments it is important to consider the contribution of the three histidines in pleurocidin, the charge state of which will have substantial impact on the interaction of the peptide with membranes of varying anionic charge density. According to the Gouy-Chapman model, the effective pH will be reduced further, the greater the anionic charge density at a membrane surface.

Molecular dynamics (MD) simulations are constructed using structures of pleurocidin or its analogues determined in anionic detergent micelles as a first approximation of the negatively charge and amphipathic surface of a bacteria plasma membrane (Supp. FIG. 1). Previously we ensured histidine residues were positively charged in molecular dynamics simulations, based on observations of pH dependent disordering of mixed zwitterionic-anionic membranes that suggested at least partial protonation at neutral pH.¹² Here we investigated pH dependent changes in conformational disorder (Supp. FIG. 2) using far-UV CD and determined no pH dependency in anionic POPG (1-palmitoyl-2-oleoyl-sn-glycero-3-phospho-(1′-rac-glycerol)) membranes in the range tested. Again however, in the mixed zwitterionic-anionic model membranes an increase in conformational disorder was observed on lowering the bulk pH. The anionic POPG bilayers are simple models of the plasma membrane of Gram-positive bacteria while the mixed zwitterionic-anionic bilayers model the corresponding structures in Gram-negative bacteria.^{14,15} Therefore, while here we have performed MD simulations both with all histidines carrying a positive or no overall charge, the simulations of pleurocidin and its analogues in anionic POPG with positively charged histidine are likely a better model of the interaction with the Gram-positive plasma membrane. In contrast the best model of the Gram-negative plasma membrane interaction lies somewhere between the simulations with the two differing charges states.

The importance of the histidine charge state in understanding the interaction with anionic or mixed anionic/zwitterionic lipid bilayers can be seen by comparing contributions from each residue in the peptide to hydrogen bonding to lipids (FIG. 1). In pleurocidin, His11 and His15 make important hydrogen bonding contributions to POPE/POPG bilayers but only when carrying a positive charge (FIG. 1D/E). When pleurocidin binds to a uniformly anionic bilayer surface the contribution from His11 is enhanced and a contribution from His23 is registered (FIG. 1F). The combination of bulk pH and anionic surface charge density therefore has the potential to offer a selectivity switch, affecting both the probability of each of the three histidines carrying a positive charge but also the nature of the non-covalent interaction between peptide and lipids—much less peptide to lipid hydrogen bonding can be expected for pleurocidin attempting to bind to a zwitterionic surface at neutral and much more when addressing an anionic surface in mildly acidic conditions. These two extremes, crudely, represent respectively the plasma membranes of a host cell and a bacterial pathogen at a site of infection.^{16,17} Interestingly, pleurocidin-VA is particularly sensitive to the charge state of the three histidines as hydrogen bonding via Lys18 is also attenuated on binding to the POPE/POPG bilayer when the histidines do not carry a positive charge. His11, when positively charged, makes a more substantial contribution to hydrogen bonding than in the parent peptide (FIG. 1J/K). In contrast, when binding to the anionic POPG bilayer, hydrogen bonding via His23 is much stronger, and via His11 much weaker, than in pleurocidin (FIG. 1F/L). Substitution of Val12 and Val16 by alanine therefore has an indirect effect on peptide-lipid hydrogen bonding that is felt in segments of the peptide that are distant from the flexible region around Gly13 and Gly17.

Substitution of arginines for the four lysines has a direct effect on peptide-lipid hydrogen bonding. Pleurocidin-KR forms approximately one and a half times as many hydrogen bonds with the bilayer as either pleurocidin or pleurocidin-VA (FIG. 1A/C).

FIG. 1. Hydrogen bonding from peptide to lipids is altered in pleurocidin analogues and affected by histidine charge state. Total (A-C) or residue specific (D-L) peptide-lipid hydrogen bonds are shown as a function of time for pleurocidin and its two analogues in three representative MD simulations; POPE/POPG bilayers with positively charged histidines (A/D/G/J), POPE/POPG bilayers with uncharged histidines (B/E/H/K) and POPG bilayers with positively charged histidines (C/F/I/L). Residue specific data is shown for pleurocidin (D/E/F), pleurocidin-KR (G/H/I) and pleurocidin-VA (J/K/L).

Notably, when the three histidines do not carry a positive charge, the difference in total peptide to lipid hydrogen bonds is greatly enhanced (FIG. 1B) indicating that selectivity of pleurocidin-KR will be diminished. The increase in hydrogen bonding can be shown to occur almost exclusively via the four arginine residues (FIG. 1G-I) and the increase in intermolecular hydrogen bonding occurs at the expense of intramolecular hydrogen bonding that would otherwise stabilize ordered α-helix conformation (Supp. FIG. 3).

FIG. 2. Secondary structure analysis of pleurocidin peptides. Top view snapshots showing ordered/disordered conformation for each of the eight peptides (A/D/G) binding to POPE/POPG bilayers in silico. From the same simulations, average psi dihedral angles and circular variance of psi are shown for each residue, averaged over 200 ns of simulation and eight peptides (B/E/H). Far-UV CD spectra obtained in anionic SDS detergent micelles or models of Gram-negative or Gram-positive plasma membranes comprising respectively POPE/POPG or POPG lipids (C/F/I). Data is shown for pleurocidin (A-C), pleurocidin-KR (D-F) and pleurocidin-VA (G-I).

Pleurocidin analogues have increased conformational flexibility in model membranes—Although pleurocidin adopts a secondary structure with high α-helix content in many membrane mimicking environments,^{11,12,18,19} in those that more closely resemble the plasma membrane of a Gram-negative bacterium, i.e. rich in a mixture of zwitterionic phosphatidylethanolamine and anionic phosphatidylglycerol, the structure becomes substantially, though not completely, disordered.^{11,12} The key interactions that determine the extent of this conformational disorder are unknown but, considering the impact of the lipid environment, are likely to include the sum of peptide—lipid interactions, notably hydrogen bonding as above but also hydrophobic effects, Coulombic interactions as well as the order of the lipid bilayer and the presence/absence of unsaturated acyl chains.

Both all-atom MD simulations and CD experiments show that the secondary structure of the two pleurocidin analogues differs substantially from that of the parent molecule (FIG. 2; Supp. FIG. 3). In contrast with our recent, analogous studies of aurein 2.5 and temporin L, where both peptides adopt ordered α-helix conformations with little flexibility evident beyond the N- and C-termini,²⁰ the present MD simulations reveal pleurocidin and its analogues exhibit substantial conformational flexibility, as evidenced by high circular variance of the psi dihedral angle, throughout the length of the peptide (FIG. 2B/E/H). The time-resolved analysis of psi dihedral angles and its circular variance can be compared with various measures of secondary structure including n−n+x hydrogen bonding, Ramachandran plots of starting and final structures, and both Dictionary of Secondary Structure of Proteins (DSSP)²¹ and DIhedral-based Segment Identification and CLassification (DISICL)²² analyses (Supp. FIG. 3). Notably, while a preference for α-helix conformation can be detected in pleurocidin in a long segment from Lys8 to Ala21, when eight peptides are considered (Supp. FIG. 4), and in segments between Lys8 and Gly13 and also Ala20-Leu25, when four peptides are considered (FIG. 2B), this is largely absent from pleurocidin-KR (FIG. 2E; Supp. FIG. 3-5). Interestingly, the preference for α-helix conformation at the C-terminus is retained and perhaps enhanced in pleurocidin-VA but α-helix conformation is lost from all residues preceding Lys14 (FIG. 2H; Supp. FIG. 3/4).

When the simulations are repeated with histidines carrying no overall charge the differences in conformational preference between the three analogues are retained although the conformational flexibility of pleurocidin around the segments of α-helix conformation is notably reduced (Supp. FIG. 3/5). The conformational flexibility of pleurocidin-KR and pleurocidin-VA is not noticeably affected by the protonation state of the three histidines. Finally, analogous MD simulations performed with an anionic, POPG membrane indicate that the membrane composition influences the conformation of the bound pleurocidin and its analogues (Supp. FIG. 6). A preference for α-helix and/or type I β-turn is now displayed throughout the length of pleurocidin, a segment with dihedral angles consistent with α-helix and/or type I 3-turn appears between Phe6 and His11 in pleurocidin-VA, concomitant with an increase in n−n+4 hydrogen bonds, but pleurocidin-KR is unaffected and little or no α-helix detected during the 200 ns of the duplicate simulations.

These data obtained from MD simulations of the first 200 ns of peptide binding are qualitatively supported by CD measurements performed in the steady state (FIG. 1C/F/I). This indicates that while all three peptides have a preference for α-helix conformation pleurocidin in both POPE/POPG and POPG models membranes the conformation of pleurocidin and, to a greater extent, pleurocidin-KR is disordered (FIG. 1 C/F). Pleurocidin-VA also adopts a disordered α-helix conformation in POPE/PG but, notably, negative bands for pleurocidin-VA at 210 and 220 nm are approximately equal and hence the disordered conformation of pleurocidin-VA differs from that of pleurocidin and pleurocidin-KR (FIG. 1I). Pleurocidin-VA adopts a more ordered α-helix conformation in POPG (FIG. 1I). CD data obtained for the all-D amino acid analogues indicate D-pleurocidin and D-pleurocidin-KR produce spectra that are mirror images of the spectra obtained for the all-L amino acid peptides (Supp. FIG. 7).

Modifications to the pleurocidin primary sequence fundamentally alter activity against model membranes—Having established that conformation and peptide-lipid hydrogen bonding is altered in pleurocidin analogues when binding to lipid bilayers, we then examined the impact of this on bilayer penetration and disruption. In both types of membranes, but most notably in those modelling the Gram-negative plasma membrane, pleurocidin-KR inserts more readily and is much more disruptive than either its parent or the pleurocidin-VA analogue.

Consistent with previous work,¹² in the 200 ns MD simulations, time-resolved insertion by all three peptides in both bilayers proceeds predominantly via the more hydrophobic N-terminus, with some penetration of the bilayer via the C-terminus segment observed, in particular in the POPG bilayers (FIG. 3A/C/E/G/I/K). The charge state of the three histidine residues is important for this process (Supp. FIG. 8). When the histidines do not carry a positive charge, pleurocidin and its analogues struggle to insert into the bilayer as effectively, with penetration in the N-terminus restricted to the first four residues and the C-terminus failing to penetrate entirely.

FIG. 3: Activity of pleurocidin and its analogues on in silico and in vitro models of bacterial plasma membranes. The depth of insertion into each membrane is shown as the Z-position for each residue, averaged over all four peptides, relative to the phosphate group plane in the upper POPE/POPG (A/E/I) or POPG (C/G/K) bilayer leaflet in six MD simulations. Positive or negative values indicate the peptides are below or above the phosphate group. Representative current traces illustrating membrane activity when DPhPE/DPhPG (B/F/J) or DPhPG (D/H/L) model membranes are challenged with each peptide at the indicated concentration (lowest concentration that induced detectable activity). Data is for pleurocidin (A-D), pleurocidin-KR (E-H) and pleurocidin-VA (I-L). Membrane disordering by each peptide in mixed POPE/POPG bilayers is shown with data obtained from ²H solid-state NMR (M/N) or from MD simulations where order parameters are calculated for all lipids within 4 Å of a peptide (O/P).

Time-resolved penetration of POPE/POPG (FIG. 3A/E/I) or POPG (FIG. 3C/G/K) bilayers differed according to peptide and bilayer composition however, with POPE/POPG bilayers providing greater discrimination. In POPG bilayers all three peptides insert into the hydrophobic core of the bilayer by the end of the 200 ns simulations with N-terminus, central and C-terminus segments all penetrating below the plane of the lipid phosphates. Some qualitative differences are apparent however with pleurocidin-KR penetrating more than pleurocidin between Gly13 and Ala19 (FIG. 3C/G) and pleurocidin-VA penetrating less than pleurocidin between Ala9 and Ala/Val12 (FIG. 3G/K). Consistent with this, electrophysiology measurements using the Port-a-Patch® automated patch-clamp system of each analogue challenging 1,2-diphytanoyl-sn-glycero-3-phospho-(1′-rac-glycerol) (DPhPG) bilayers (FIG. 3D/H/L) indicate all three peptides induce ion conductance activity at comparable peptide concentrations. Again, some qualitative differences are apparent; while all three peptides generate a mix of irregular but also channel like activities, the latter was consistently observed more frequently for pleurocidin-KR. Pleurocidin and, in particular pleurocidin-VA, were consistently more likely to trigger bursts of conductance of varying amplitude which lacked discrete opening levels.

The interaction of pleurocidin and its two analogues with POPE/POPG bilayers differs substantially. In MD simulations, much greater penetration of the hydrophobic core was observed between Lys/Arg7 and His15—the region where hydrogen bonding is enhanced by three of the four lysine to arginine substitutions—for pleurocidin-KR over pleurocidin (FIG. 3 A/E). Insertion by pleurocidin-VA is more like that of pleurocidin but penetration of Lys14/His15 is weaker and more reliant on Phe5/Phe6 at the N-terminus. Disordering of the lipid acyl chains was also monitored in the MD simulations (FIG. 3O/P; Supp. FIG. 9) as well as by ²H solid-state NMR of chain deuterated POPG (FIG. 3M) or POPE (1-palmitoyl-2-oleoyl-sn-glycero-3-phosphoethanolamine) (FIG. 3N). In MD simulations, AMPs have been shown to disorder lipids that are close to the peptide while those that are more distant become more ordered.^{23,24} Such data, from the first 200 ns of a peptide-bilayer interaction, will not perfectly correlate with ²H NMR data obtained in the steady state. Nevertheless, both methods indicate pleurocidin-KR induces greater disorder than pleurocidin or pleurocidin-VA although manifested in the zwitterionic POPE component in the MD simulations and in the anionic POPG component by NMR (FIG. 3O/M). Electrophysiology measurements reveal both lysine to arginine and valine to alanine substitutions fundamentally alter membrane activity in 1,2-diphytanoyl-sn-glycero-3-phosphoethanolamine (DPhPE)/DPhPG models of the Gram-negative plasma membrane. Challenge with pleurocidin induces irregular and high intensity activity, with no evidence of channel like activity, that gradually subsides with the bilayer remaining intact (FIG. 3B). This is consistent with pleurocidin inducing conductance as it passes from one leaflet of the bilayer to the other and with its known ability to penetrate within bacteria as part of its bactericidal activity. In contrast, challenge with pleurocidin-KR reproducibly leads to a gradual increase in, again, irregular conductance, until the bilayer breaks (FIG. 3J). Conductance in the DPhPE/DPhPG membrane induced by pleurocidin-VA was inconsistent. Frequently very high peptide concentrations are required to trigger activity and often no activity is detected at all. When activity is observed it is irregular and with high intensity.

Pleurocidin analogues offer species and environmental dependent improvements in anti-bacterial potency, broadening the spectrum of activity—Next we investigated whether there is evidence for the altered mechanism of action altering anti-bacterial activity, first through assessing potency in vitro against a panel of Gram-negative or Gram-positive pathogens using the AMPs either alone (Table 2) or in combination with clinically relevant antibiotics (Table 3) and, second, through an NMR metabolomic study of how bacteria respond to challenge with the all D-amino acid pleurocidin and its -KR or -VA analogues. Anti-bacterial susceptibility testing was conducted both in Mueller Hinton Broth (MHB), as recommended,²⁵ and also in Roswell Park Memorial Institute 1640 (RPMI) medium. The latter was used since recent studies have shown that both false-negative²⁶ and false-positive²⁷ results can arise from exclusively testing antibiotic susceptibility in MHB alone.

In MHB, pleurocidin possesses broad spectrum activity. However, with the notable exception of Pseudomonas aeruginosa, activity against Gram-negative isolates is generally higher than that against Gram-positive. The potency of pleurocidin-KR is marginally greater than that of pleurocidin against most Gram-negative isolates but against P. aeruginosa and Gram-positive isolates the improvement is more substantial. The potency of pleurocidin-VA is comparable to the parent peptide against Gram-negative isolates but reduced against Gram-positive isolates. Most notably however, the in vitro cytotoxicity of pleurocidin-VA against four mammalian cell lines is severely attenuated. Analogues composed entirely of D-amino acids do not gain antibacterial potency over their all L-amino acid enantiomers in MHB, suggesting there is little to no difference in their mechanism of action. None of the analogues, whether comprising L- or D-amino acids demonstrate noticeable hemolysis at the concentrations where they are effective antibacterials, and the therapeutic index over red blood cell haemolysis will be at least 100-fold for D-pleurocidin-KR.

FIG. 4: NMR metabolomics identifies altered mechanism of action in D-pleurocidin analogues. Volcano plots obtained from liquid state ¹H NMR of spent bacterial culture reveal the metabolic strategy of EMRSA-15 in MHB (A), challenged with D-pleurocidin (G), D-pleurocidin-KR (H) or D-pleurocidin-VA (I), or in RPMI (B). The major catabolic pathways of EMRSA-15 are shown for context with key enzymes formate dehydrogenase (FDH), succinate dehydrogenase (SDH), nitrate reductase (NR), nitrite reductase (NiR) (F). ¹H HR-MAS NMR of bacterial pellets reveals the effect on cellular metabolites of growth in these media (C) or challenge with antibiotics in MHB (J-L).

Production of ROS/RNS when challenged by antibiotics, as monitored by 2′-7′dichlorodihydrofluorescin diacetate (DCFH-DA), is shown for MHB (D) and RPMI (E).

Performing the same experiments with RPMI (with 5% FCS) in place of MHB alters the susceptibility pattern considerably with Gram-negative and Gram-positive isolates affected differently and greater discrimination between L- and D-enantiomers. Although, Gram-negative isolates are generally less susceptible in RPMI when compared with MHB, the D-enantiomers better retain their activity, with D-pleurocidin-KR notably remaining effective at 4 μg/ml or less for all but the P. aeruginosa isolates. In contrast, the potency of the pleurocidin analogues in RPMI is most commonly enhanced against Gram-positive isolates, when compared with MHB, with D-enantiomers now gaining an advantage. This improvement is most noticeable for D-pleurocidin such that its disadvantage with respect to D-pleurocidin-KR, in these conditions, is negligible. Nevertheless, the selectivity index reveals D-pleurocidin-KR as the analogue with the greatest therapeutic potential for EMRSA-15, an isolate for which a murine lung infection model has been established in our laboratory.

Testing of combinations of D-pleurocidin or D-pleurocidin-KR with clinically useful antibiotics was also assessed in vitro in both MHB and RPMI (Table 3). Both peptides act in synergy with the aminoglycoside tobramycin or with rifampin. This effect was most noticeable when Pseudomonas aeruginosa RP73 was tested in RPMI and the same bactericidal activity is achieved with eight times less tobramycin or rifampin as is achieved without the peptide adjuvant.

While the ability of pleurocidin both to damage bacterial plasma membranes and penetrate within bacteria to access intracellular targets is established, the relative contributions of these properties to its bactericidal activity against different bacterial species may vary and may also be influenced by growth conditions. Assays with fluorescent reporter dyes have revealed that hydroxyl radical increases and membrane permeabilization following challenge with pleurocidin at its MIC vary substantially.²⁸ Killing of Escherichia coli ATCC 25922 or S. aureus ATCC 25923 involved substantial oxidative stress but little membrane permeabilization. In contrast, much more permeabilization of P. aeruginosa ATCC 27853 and Enterococcus faecium ATCC 19434 was induced by pleurocidin, in addition to oxidative stress. To test whether mechanisms of action distinct from that of D-pleurocidin are adopted by its analogues and to better understand the role of bacterial metabolism in susceptibility we performed an NMR metabolomic study (FIG. 4; Supp. FIG. 11-25). By culturing either EMRSA-15 or P. aeruginosa RP73 in the presence or absence of each D-pleurocidin analogue, we aim to infer differences in their bactericidal strategy from the measures the bacteria take to overcome the challenge.

In MHB, as monitored by ¹H NMR, EMRSA-15 metabolism is a mix of fermentation, aerobic and anaerobic respiration. The production of formate, lactate and ethanol indicate threonine, serine and glycine and what little glucose is present mostly feed mixed-acid fermentative pathways, consistent with acidification of the spent media (FIG. 4A; Supp. FIG. 10/12).³⁰ Uridine consumption and uracil production are associated with peptidoglycan biosynthesis while uracil is known to be essential for anaerobic growth.^{29,31} Succinate excretion to the media is consistent with anaerobic respiration, with fumarate acting as an electron acceptor.

The effect on this process, of challenging EMRSA-15 with sub-inhibitory D-pleurocidin, is profound (Supp. FIG. 12) and results in both a fundamental change in metabolic strategy and in cellular metabolite composition (FIG. 4G; Supp. FIG. 13). Fermentation of serine, glycine and glucose, but not threonine, is stopped following challenge with D-pleurocidin and the acidification of the spent culture is reduced (Supp. FIG. 10). Excretion of ornithine, succinate, ethanol, lactate, leucine, methionine and 2-aminobutyrate consequently halts while consumption of uridine is also stopped. Consumption of adenosine, acetate and aspartate increases while the challenge initiates consumption of lysine, arginine, tyrosine, and valine. Lysine and valine feed into the TCA cycle via respectively α-ketoglutarate and succinyl-CoA. Consumption of glutamate and isoleucine from the media is reversed and these amino acids are instead excreted while formate and phenylalanine are consumed instead of being excreted. Within the cell, succinate and uracil are depleted, as are choline, citrulline and valine while acetate and betaine increase. Consumption of tyrosine and phenylalanine (via the homogentisate pathway described for Pseudomonas putida³²) and aspartate can all be associated with production of fumarate. Therefore, both fumarate and nitrate might be expected to be important anaerobic electron acceptors.²⁹ However, the halt of succinate excretion and its intracellular depletion points to a potential switch from fumarate to nitrate as an anaerobic electron acceptor. The concentration of the latter in MHB is unknown and is invisible to the NMR method, but DCFH-DA fluorescence, which is sensitive to reactive oxygen (ROS) and reactive nitrogen species (RNS), increases substantially (p<0.001) (FIG. 4D). Taken together, EMRSA-15 growing in the presence of D-pleurocidin is unable to use fermentation and instead relies more heavily on anaerobic respiration, while the TCA cycle is active but fed from new carbon sources.

The effects of both D-pleurocidin analogues on EMRSA-15 can be distinguished from the parent peptide (FIG. 4D/H/I). Unlike D-pleurocidin, neither D-pleurocidin-KR nor D-pleurocidin-VA affect fermentation to any great extent (FIG. 4H/I; Supp. FIG. 12). D-pleurocidin-KR and D-pleurocidin-VA can be distinguished based on increased acetate consumption and alanine, glutamate and histidine excretion for the latter. The response of the cellular metabolite composition is more revealing, with the D-pleurocidin-VA treatment resembling more closely the effect of D-pleurocidin challenge on EMRSA-15 (FIG. 4J/L). Following challenge with sub-inhibitory D-pleurocidin-KR, eight times more potent than D-pleurocidin in MHB, there is no depletion of choline, citrulline or valine, that of succinate is mitigated and uracil is instead increased. The levels of acetate and betaine are as that observed for the unchallenged condition, but aspartate is depleted, and lactate, leucine and isoleucine levels are increased alongside plasma membrane lipid unsaturation (FIG. 4G; Supp. FIG. 13). The mechanism of action of the more potent D-pleurocidin-KR, as inferred from changes in EMRSA-15 metabolism, is therefore fundamentally different from that of the parent D-pleurocidin in MHB and may depend on a greater contribution from membrane damage.

In glucose rich RPMI, while ethanol is still produced, the fermentative production of lactate by EMRSA-15 is much more substantial such that the media is acidified still further, potentially triggering stress (FIG. 4B; Supp. FIG. 1), and little or no formate is produced. Both fermentation and anaerobic respiration may again be expected with RPMI containing 2 g/litre glucose and 100 mg/litre calcium nitrate as a potential electron acceptor. As well as glucose, notably 300 mg/litre glutamine is available, and its consumption is also preferred (FIG. 4B). This might ensure that the TCA cycle is being fed via 2-oxoglutarate but may also reflect the roles of glutamine as a major nitrogen donor, in osmotic protection and in multiple steps of peptidoglycan synthesis.^{33,34} The effect of this change in metabolic strategy for the cellular metabolite composition is substantial (FIG. 4C) and may render the bacteria more susceptible to the action of D-pleurocidin and its analogues. In contrast with their effect in MHB, apart from a halt in proline and histidine excretion, the three analogues have only a modest impact on the metabolism of EMRSA-15 in RPMI (Supp. FIG. 15). Glucose consumption and lactate and ethanol excretion are largely unaffected and the increase in DCFH-DA fluorescence on D-pleurocidin challenge is absent (FIG. 4E). The impact of the three analogues on the cellular metabolite composition is similar (Supp. FIG. 16). As observed for D-pleurocidin challenge of EMRSA-15 MHB above, succinate, uracil and adenosine are depleted, and cellular acetate increases following challenge with all three analogues. Reflecting the similarity in their MIC in RPMI, apart from a modest increase in plasma membrane lipid unsaturation and a depletion of 2-aminobutyrate and lysine, there is therefore much less to distinguish the action of D-pleurocidin-KR from its parent.

A similar study was performed for P. aeruginosa RP73. There is little evidence of fermentation for P. aeruginosa RP73 when cultured in MHB or RPMI, where there is notable consumption of, respectively, formate and lactate. This strain was isolated from a cystic fibrosis patient where consumption of lactate is characteristic.³⁵ Lactate is a substrate for a family of lactate dehydrogenases which support anaerobic or aerobic metabolism and is present in the added FBS which is essential for growth of P. aeruginosa in RPMI.³⁵ In both media therefore a more substantial role for anaerobic respiration is expected, albeit supported by differing electron donor and acceptor pairs.

Consequently, while an increase in DCFH-DA fluorescence is again detected on D-pleurocidin challenge of P. aeruginosa RP73 in MHB and modest changes in metabolite consumption are detected, there is no accompanying fundamental shift in metabolic strategy (Supp. FIG. 18). Indeed, no fundamental change in metabolism is induced by challenge with either D-pleurocidin or D-pleurocidin-KR or tobramycin. In RPMI, challenge with sub-inhibitory concentrations of D-pleurocidin-KR, but not D-pleurocidin, induces a substantial increase in DCFH-DA fluorescence emission and also in the intensity of resonances assigned to lipid-CH₃, lipid-CH₂ and lipid—CH═CH—while there are key differences in the metabolic perturbations it induces; most notably, isoleucine, leucine, methionine, phenylalanine, tyrosine and valine are all consumed less, relative to D-pleurocidin challenge, while arginine consumption continues and may increase, excretion of acetate substantially decreases while that of aspartate, glutamate and ornithine continues and may increase and, lysine excretion is triggered (Supp. FIG. 19/24). A similar but more muted trend in membrane remodelling is observed for P. aeruginosa RP73 challenged in MHB (Supp. FIG. 19/21). From these data, it may again be inferred that the impact of D-pleurocidin-KR is felt more keenly at the plasma membrane when compared with its D-pleurocidin parent. For P. aeruginosa RP73 however, while differences in response to the two D-pleurocidin analogues can be detected, which again suggest they operate using distinct mechanisms the effect on antibacterial potency is minor.

D-pleurocidin-KR is an effective therapeutic in a murine model of EMRSA-15 lung infection—Having identified that the enhanced membrane disrupting capabilities of D-pleurocidin-KR renders it less sensitive to infection setting dependent changes in bacterial metabolism and since the selectivity index for inhibition of EMRSA-15 relative to HEK293 cellular toxicity was greatest for D-pleurocidin-KR of all the analogues tested, we advanced D-pleurocidin-KR to an established murine model of EMRSA-15 infection (FIG. 5).

D-pleurocidin-KR is readily soluble in aqueous media and amenable to intravenous delivery. A dose of 1×10⁶ colony forming units (cfu)/mouse EMRSA-15 in tryptic soy agar beads inoculated in the lung establishes a stable infection (4.26 t 0.24 log 10 CFU/ml at 4h; 4.43 t 0.97 log 10 CFU/ml at 48h) which, if untreated, causes approximately a 5% loss of weight in 48 hours (FIG. 5B). Treatment with a cumulative dose of 15 or even 1.5 mg/kg/48h D-pleurocidin-KR mitigates this weight loss to a similar extent to that achieved with 600 mg/kg vancomycin (a similar reduction may be achieved with 200 mg/kg vancomycin, data not shown), although the lowest dose of 0.15 mg/kg D-pleurocidin-KR has no observable effect (FIG. 5B). The two higher doses also achieve a significant reduction in CFU with a 1.14 log₁₀ reduction for the highest dose approximately equal to that achieved with 600 mg/kg vancomycin (FIG. 5A) (with 1.1 and 0.9 log 10 reduction achieved respectively for 15 and 1.5 mg/kg/48h doses, comparable with that achieved with vancomycin (FIG. 5A)). D-pleurocidin-KR was tolerated at 15 mg/kg/24h when administered i.v. to ICR mice (data not shown) indicating its in vivo therapeutic index is approximately 20-fold, before dose optimisation. The effect of D-pleurocidin-KR therapy on the innate immune response was also characterised through analysis of bronchoalveolar lavage fluid (BAL) (FIG. 5C-I). The mouse keratinocyte chemoattractant (KC), equivalent to CXCL1 in humans, response is similar to that observed for lung CFU with infection stimulating an increase in this cytokine and treatment with vancomycin or the two higher D-pleurocidin-KR doses causing KC levels to fall to that of the uninfected group (FIG. 6H). Both IL-6 and neutrophil recruitment (reflected also in total cell numbers; FIG. 5C) are reduced following therapy with either vancomycin or D-pleurocidin-KR. However, a significant reduction is also observed for the lowest dose of D-pleurocidin-KR (FIG. 5D/F), which did not cause a significant reduction in lung CFU or mouse KC. No effects of vancomycin and D-pleurocidin-KR therapy could be detected on macrophage numbers or levels of TNFα or MCP-1 in BAL (FIG. 5E/G/I). Mouse KC is sensitive to EMRSA-15 lung infection while IL-6 reflects neutrophil recruitment and these data indicate D-pleurocidin-KR effectively reduces the burden of lung infection and may have a dampening effect on neutrophil recruitment but we do not observe evidence of a substantial innate immune response.

FIG. 5: Systemically delivered D-pleurocidin-KR is effective in a murine model of EMRSA-15 lung infection. C57Bl6J mice, challenged with 1×10⁶ cfu/mouse EMRSA-15 in tryptic soy agar beads, were treated with vancomycin or D-pleurocidin-KR in three intravenous doses at 4, 24 and 30 hours post infection to achieve the cumulative doses indicated. Bacterial burden in the lung (A), weight loss over the 48-hour infection period (B) and BAL cells (C-E) and cytokines (F-I) reveal the effect of each intervention. Significance is indicated relative to the saline vehicle (p<0.05 *; <0.01 **; <0.001 ***; <0.0001 ****).

An analogous experiment was performed to determine whether synergy, observed in vitro between D-pleurocidin-KR and tobramycin when challenging P. aeruginosa RP73, translated to the murine model (Supp. FIG. 26). In this model, a cumulative dose of 200 mg/kg tobramycin delivered over 30 hours produces a 1.4 log₁₀ reduction in lung CFU. Reducing the tobramycin dose 8-fold to 25 mg/kg produces only a 0.8 log₁₀ reduction (p=0.0919) while combining the same dose with 1.5 mg/kg D-pleurocidin-KR modestly improves the effect and causes a 1.2 log₁₀ reduction (p=0.0016). The combination of 25 mg/kg tobramycin and 15 mg/kg D-pleurocidin-KR produces only a 0.6 log₁₀ reduction in CFU which is 0.8 log₁₀ less than that obtained with 200 mg/kg tobramycin (p=0.0411). There is therefore no dose dependent improvement in the adjuvant behavior of D-pleurocidin-KR and higher doses may actively impair the observed effect.

### Discussion

A list of twenty-seven clinical and nine preclinical studies of AMPs, published in May 2019, shows there is considerable appetite for developing peptide-based molecules as potential therapeutics.³⁶ Of these thirty-six AMPs however, only seven are/were being developed for intravenous delivery. Of these seven, three have been discontinued, one (POL7080) is specific to the P. aeruginosa LPS-assembly protein LptD and three (EA-230, Ghrelin and p2TA (AB103)) function via immunomodulation. Only one, hFF1-11, a lactoferricin derivative has shown direct antimicrobial activity through membrane disruption but the in vivo efficacy may also be related more to its immunomodulatory capabilities which include the release of pro-inflammatory cytokines and stimulation of monocyte differentiation.³⁷⁻³⁹ As a result, with few if any bactericidal AMPs finding i.v. applications, a recent, authoritative review has expressed the opinion that “AMPs may never be able to achieve the same clinical outcomes as conventional antibiotics” with neither naturally occurring nor rationally designed AMPs sufficiently potent.⁴⁰ Helpfully, Haney et al identify key areas where improved understanding may nevertheless contribute to more fruitful translation of AMPs into useful therapeutic agents. These include: 1) the perception that each individual amino acid residue may have an important role and that no single active conformation exists; 2) specific to AMPs that access the bacterial cytoplasm, better models of bacterial plasma membrane translocation concomitant with, or in the absence of, permeabilization; 3) determining what features of a given infection setting are reproduced by each in vitro assay and; 4) the concept that exogenous AMPs are unlikely to act in isolation and may act in synergy with the host innate immune system (and also clinically relevant antibiotics).

We have shown that analogues of pleurocidin, in particular D-pleurocidin-KR, are potent bactericidal AMPs which can be delivered intravenously to treat bacterial lung infections without triggering the release of pro-inflammatory cytokines or stimulating recruitment of innate immune cells in the mouse model. The modification strategy ensures that in some cases a sixteen-fold improvement in potency is observed for D-pleurocidin-KR over pleurocidin and this compares favourably with e.g. efforts to obtain shortened pleurocidin analogues. Furthermore, the bactericidal activity of pleurocidin can be rendered less sensitive to changes in bacterial metabolism though manipulation of its interaction with lipid bilayers; even relatively minor modifications are sufficient to ensure an altered mechanism of action. Both D-pleurocidin and the D-pleurocidin-KR analogue can act in synergy with the aminoglycoside tobramycin or rifampin in vitro and there is therefore some promise that AMPs may find application as adjuvants to existing, clinically relevant antibiotics. This may be achieved in particular by reducing the risk of resistance emerging, either via manipulation of see-saw mechanisms⁴¹ or through an improved pharmacodynamics profile.⁴²

A molecular level understanding of selectivity, membrane translocation and disruption—the MD simulation and patch clamp studies, using bacterial plasma membrane models, offer a mutually supporting view of the variety of ways that pleurocidin AMPs may interact with the plasma membrane of differing bacteria. In the context that pleurocidin is known to both disrupt the bacterial plasma membrane but also translocates to seek intracellular targets,^{6,9,10} observations from patch-clamp studies identify membrane activity consistent with both of these properties. The highly irregular but high amplitude conductance that diminishes over time, observed for pleurocidin most notably in membranes that model the Gram-negative bacterial plasma membrane and remain intact, is consistent with an AMP crossing the bilayer without major structural disruption. Such activity is also observed in models of Gram-positive plasma membranes where, additionally, more channel like conductance is observed. Taken together with previous fluorescence studies that reveal species and even strain dependent differences in the extent of membrane permeabilization caused by pleurocidin at its minimum inhibitory concentration,²⁸ this suggests that a primary mechanism of action will be to penetrate the bacteria but at higher concentrations a secondary, membrane disruptive, effect will be observed. While differences between bacterial species and strains, and also their environment will impact on which of these two effects contributes most to bacterial death, it is shown here that the interaction between peptide and lipid bilayer can be manipulated to increase the role of membrane damage in the bactericidal strategy. Notably, the MD simulations, supported by CD spectroscopy, show increasing hydrogen bonding between the bilayer and the peptide (pleurocidin-KR) produces much greater conformational flexibility and consequently disordering of the bilayer. This highlights the importance of peptide-lipid hydrogen bonding in modulating the interaction of pleurocidin with bilayers of differing composition and shows how this, and the charge state of the three histidines, is critical in determining the outcome. Positively charged histidines enjoy favourable Coulombic interactions with anionic lipids which will also mitigate unfavourable interactions between histidines and lysines that might be positioned close in space, thus affecting not only binding to surfaces of varying anionic charge but also modulating the preferred conformation. Further, the hydrogen bonding potential of positively charged histidine is greater. This, together with varying possibilities for hydrogen bonding for lipids common in prokaryotic (phosphatidylglycerol and phosphatidylethanolamine—a primary amine) and eukaryotic (phosphatidylcholine—a quaternary amine—is more common) highlights the role of histidine as a selectivity switch, influencing the likely therapeutic index of these compounds. In the absence of histidine mediated hydrogen bonding, the interaction of pleurocidin-VA with less anionic bilayers is much weaker than that of the parent in the centre and C-terminal segments. Further manipulation of this property may offer additional means of improving selectivity.

The distinct mechanisms of action of D-pleurocidin and D-pleurocidin-KR highlights two aspects of AMP development that may be important in successful translation. If it is possible to fundamentally alter the mechanism of action within members of an SAR series with only a few amino acid substitutions, then it is likely that exogenous AMPs will differ in mechanism from endogenous HDPs, making additive relationships less likely. Further, although exogenous AMPs may share some characteristics with endogenous HDPs, it can be expected that the extent of cross resistance but will be mitigated by significant differences in mechanism of action.

Altered mechanism of action, sensitivity to bacterial metabolic strategy and prospects for infection setting specific therapeutic outcomes—There is increasing recognition that susceptibility testing in bacteriological media may both under- or over-estimate the potency of different classes of antibiotics and is unlikely to replicate the conditions encountered in vivo.^{26,27} In spite of well-established protocols for susceptibility testing of antibiotics, including AMPs,²⁵ the methodology used to determine antibacterial potency of AMPs remains highly variable.⁴⁰ The use of mammalian cell culture media has been presented as a move towards better replicating the infection setting and presents AMPs with some notable challenges including increased ionic strength and the presence of serum proteases. Altered growth conditions may also affect the production of virulence factors.^{43,44} The D-enantiomers of pleurocidin and its analogues did not outperform their L-amino acid parent molecules until RPMI (5% FBS) was used in place of MHB. The need to modify linear AMPs to avoid proteolytic degradation in more challenging conditions is therefore clear.

While mammalian cell culture media may present greater challenges to AMPs, and this is notable in their activities against Gram-negative isolates, bacterial metabolic activity may also affect antibiotic outcomes⁴⁵ and it is of considerable interest that, irrespective of stereochemistry, pleurocidin analogues are more potent against many of the Gram-positive isolates than their parent molecules. Further, the difference in potency between D-pleurocidin and D-pleurocidin-KR against EMRSA-15 disappears when susceptibility testing is performed in RPMI with both peptides more potent than when evaluated in MHB. EMRSA-15 growth in MHB adapts to the presence of D-pleurocidin by shutting down fermentation and placing greater reliance on anaerobic respiration, most likely using nitrate as an electron acceptor. This response is not observed when EMRSA-15 is challenged with the more potent D-pleurocidin-KR. This can be attributed to the lower concentration of D-pleurocidin necessarily used for the challenge and/or a fundamentally altered mechanism of action. The latter is supported by evidence that EMRSA-15 responds to D-pleurocidin-KR, which carries substitutions which cause greater disruption of model bilayers, by remodelling its plasma membrane. While it is not clear whether the gain in membrane disruption in D-pleurocidin-KR occurs at the expense of the ability to penetrate the bacterial cytosol, this property appears crucial in reducing sensitivity to the bacterial metabolic strategy. Although the increased dependence on fermentation can be implicated in rendering EMRSA-15 more sensitive to the action of D-pleurocidin analogues, the origin of their increased potency against EMRSA-15 in RPMI (5% FBS) is not completely clear but is nonetheless welcome and suggests that, if EMRSA-15 adopts a fermentative metabolic strategy in a lung infection setting then, all D-pleurocidin analogues will be more potent than anticipated and there is little advantage to selecting D-pleurocidin-KR. However, although the RPMI (5% FBS) culture conditions have been suggested as a better means of reproducing the bacterial metabolic strategy in vivo, infection setting dependent variation in nutrient availability can be expected to affect therapeutic outcomes.³⁰ Indeed, although fermentable substrates, including glucose are abundant in the lung infection setting, a recent study of S. aureus gene expression during cystic fibrosis lung infections finds that expression of genes involved in fermentation and use of nitrate as an electron acceptor is low. This would then predict a greater chance of therapeutic success for D-pleurocidin-KR over D-pleurocidin. The varying sensitivity of the D-pleurocidin analogues to bacterial metabolism is further evidence that efforts, to improve our understanding of bacterial metabolism in different infection settings,^{44,46} need to be recognized when designing in vitro susceptibility testing if this is to improve success in pre-clinical studies. The present study however suggests that including a range of conditions in in vitro susceptibility testing, that stimulate different metabolic strategies in target bacteria, may be important in selecting AMPs that are resilient and improving success of AMPs in pre-clinical studies.

Exogenous bactericidal AMPs can function in parallel with the innate immune system—the immunomodulatory abilities of HDPs are increasingly recognized as being critical to their role in the innate immune system. HDPs have been shown to be capable of inducing or modifying production of cytokines or chemokines as well as inhibiting pro-inflammatory responses from host cells which might arise from bacterial components including lipoteichoic acid, peptidoglycan, lipopolysaccharide and bacterial DNA.⁴⁷ This has led to the design of small synthetic peptides, focusing on enhancing their immunomodulatory capability,⁴⁸ or enhancing the immunomodulatory capability of bactericidal peptides identified in nature.⁴⁹ Analysis of BAL fluid shows no evidence of any increased cytokine response or recruitment of either neutrophils or macrophage following i.v. D-pleurocidin-KR administration at doses that effectively reduce EMRSA-15 load in the lung. The dampening of IL-6 levels and neutrophil recruitment without any significant reduction in lung CFU does suggest that D-pleurocidin-KR can inhibit pro-inflammatory responses either directly or indirectly. However, since D-pleurocidin-KR is a highly potent (in vitro MIC 0.5 μg/ml/0.18 μM), bactericidal AMP against EMRSA-15 and, with no evidence of an immune-stimulatory effect in vivo, its therapeutic effect must currently also be primarily ascribed to a direct effect on bacteria at the site of infection.

### Conclusion

With appropriate modification and an understanding of the requirements for bactericidal activity that is robust in the face of different bacterial metabolism and more challenging environmental conditions, pleurocidin analogues are effective in treating an EMRSA-15 lung infection. Although pleurocidin may represent a special case, our results suggest that, despite widely held concerns, bactericidal AMPs may be suitable for development for intravenous delivery and systemic therapeutic applications.

### Methods

Peptides and lipids. Pleurocidin, pleurocidin-KR, pleurocidin-VA and their all D-amino acid analogues were purchased from Cambridge Research Biochemicals (Cleveland, UK) as desalted grade (crude). Further materials are as described previously with MICs generated from at least three biological replicate experiments.²⁰

Antibacterial activity assay. The antibacterial activity of the peptides was assessed through a modified two-fold broth microdilution assay²⁵ as described previously.²⁰ Synergy was measured using standard microdilution checkerboard assays under the same conditions as the MICs (Fratini F, Mancini S, Turchi B, Friscia E, Pistelli L, Giusti G & Cerri D. (2017) A novel interpretation of the Fractional Inhibitory Concentration Index: The case Origanum vulgare L. and Leptospermum scoparium J. R. et G. Forst essential oils against Staphylococcus aureus strains. Microbiol. Res. 195, 11-17.). The fractional inhibitory concentration was calculated from the most synergistic well on the plate for three independent repeats, and presented as the average +/−standard deviation. FIC is calculated as (MIC of compound A in combination with B/MIC of compound A alone)+(MIC of compound B in combination with A/MIC of compound B alone). MICs were determined on the same plates as the FICs to increase reproducibility. FIC values ≤0.5 were considered strongly synergistic, and 0.5-<1 were weakly synergistic.

Cytotoxicity assay. HeLa and HEK293 cell lines were purchased from ECACC and cultured in Eagle's Minimum Essential Media (EMEM) containing glutamine, supplemented with 10% FBS and 1× non-essential amino acids (NEAA) at 37° C., 10% CO₂. Cytotoxicity was measured by incubating starter cultures of cells for 24 hours in a 96 well plate, gently removing the supernatant and replacing it with dilutions of peptides and controls, incubating for a further 24 hours and then staining of the cells with the second generation tetrazolium dye, XTT. Cells were stained with 0.2 mg/ml XTT and 5 mM N-methyl dibenzopyrazine methyl sulfate (PMS), incubated for 4 hours and the IC₅₀ was calculated from the OD₄₇₅ which was measured using a Clariostar plate reader. Data presented are averages and standard error from three biological replicate experiments.

Haemolysis assay. Haemolysis was tested by incubating titrations of AMPs in PBS with freshly collected human red blood cells for 1 hour at 37 C. Control wells were treated with 0.1% Triton-X-100 to ensure complete lysis, or PBS-only to represent no lysis. Samples were spun down to remove non-lysed cells and the OD⁵⁵⁰ of the supernatant was measured using a Clariostar plate reader. The percentage of haemolysis was calculated as % haemolysis=(A_{P}−A_{B})/(A_{C}−A_{B})×100, where A_{P} is the absorbance value for a known peptide concentration, A_{C} is the absorbance of the 0.1% Triton-X-100 control, AB is the absorbance of the PBS control. Data presented are averages and standard error from three biological replicate experiments.

NMR structure determination. The NMR samples consisted of a 0.5 mM peptide solution also containing 50 mM deuterated sodium dodecyl sulphate (SDS-d₂₅) with 5 mM Tris(hydroxymethyl-d₃)-amino-d₂-methane buffer at pH 7.10% D₂O containing trimethylsilyl propanoic acid (TSP) was added for the lock signal and as internal chemical shift reference. The temperature was kept constant at 310 K during the NMR experiments. NMR spectra were acquired on a Bruker Avance 800 MHz spectrometer (Bruker, Coventry, UK) equipped with a cryoprobe. Standard Bruker TOCSY and NOESY pulse sequences were used, with water suppression using a WATERGATE 3-9-19 sequence with gradients (mlevgpphi9 and noesygpphi9). The ¹H 90 degree pulse was calibrated at 37.04 kHz. The TOCSY mixing time was 90 ms, and the mixing time for the NOESY spectra was set to 150 ms. The relaxation delay was 1 s. 2048 data points were recorded in the direct dimension, and either 256 or 512 data points in the indirect dimension. The spectra were processed using Bruker TOPSPIN. The free induction decay was multiplied by a shifted-sine² window function. After Fourier transformation, the spectra were phase corrected, a baseline correction was applied, and spectra were calibrated to the TSP signal at 0 ppm.

CARA⁵⁰ (version 1.9.1.2) and Dynamo⁵¹ software were used for assignments and structure calculation, respectively and the structure determination was as described previously.^{20,51} Structural coordinates were deposited in the Protein Data Bank (www.rcsb.org) and Biological Magnetic Resonance Bank (BMRB; www.bmrb.wisc.edu) under accession codes of 6RSF and 6RSG (PDB) and 34404 and 34405 (BRMB) for pleurocidin-KR and pleurocidin-VA respectively.

Molecular dynamics simulations. Simulations were carried out on either the ARCHER Cray XC30 supercomputer, or Dell Precision quad core T3400 or T3500 workstations equipped with a 1 kW Power supply (PSU) and two NVIDA PNY GeForce GTX570 or GTX580 graphics cards using Gromacs.⁵³ The CHARMM36 all-atom force field was used in all simulations⁵⁴⁻⁵⁶. All simulations were run, as described previously²⁰ and for a total of 200 nanoseconds and repeated twice, with peptides inserted at different positions and orientations, giving a total of approximately 4.0 μs simulation. Liposome preparation and circular dichroism (CD) spectroscopy. Small unilamellar vesicles (SUV) were produced and far-UV spectra of the peptides obtained as described previously²⁰ using a Chirascan Plus spectrometer (Applied Photophysics, Leatherhead, UK) with samples maintained at 310 K. For liposome experiments the lipid concentration was 5 mM and a peptide to lipid molar ratio of 1:100 was maintained while the SDS micelles concentration was 20 mM with 200 μM peptide. Electrophysiology experiments (Patch-clamp). Patch clamp experiments were performed using the Port-a-Patch® (Nanion Technologies GmbH, Munich, Germany) with giant unilamellar vesicles (GUVs) composed of DPhPE/DPhPG (60:40, mol:mol) or DPhPG prepared by the electroformation method using the Nanion Vesicle Prep Pro setup (Nanion Technologies GmbH, Munich, Germany) as described previously.²⁰

Metabolomics sample preparation—S. aureus EMRSA-15 and P. aeruginosa RP73 were streaked on Mueller Hinton agar plates and RPMI (supplemented with 5% foetal bovine serum (FBS)) agar plates and incubated at 37° C. overnight or until single colonies formed. On three separate occasions, three single colonies were used to inoculate 10 ml of respective media and challenges set up in broth using subinhibitory concentrations of: D-pleurocidin, D-pleurocidin-VA, D-pleurocidin-KR or the antibiotic tobramycin. Cultures were incubated overnight at 37° C. without shaking and pelleted by centrifugation at 4° C. The supernatant was removed and filtered separately for spent media analysis, the pellet was briefly washed three times with phosphate buffered saline (PBS). The pellets were frozen in liquid nitrogen and lyophilised overnight in an Alpha 1-2 LD plus freeze dryer (Martin Christ, Germany) and stored at −20° C. until required. Samples were transferred into HR-MAS inserts for use in 4 mm Bruker MAS rotor and resuspended in 30 μl of D₂O containing 3-(Trimethylsilyl)propionic-2,2,3,3-D₄ acid sodium salt (TMSP-2,2,3,3-D₄). The removed supernatant was filtered through 0.2 μm filters and pH adjusted to within 0.3 pH units within the same sample set. The samples were supplemented with 10% D₂O containing TMSP-2,2,3,3-D₄ and loaded into 5.0 mm NMR tubes.

HR-MASNMR—NMR spectra were collected at 600 MHz for ¹H on a Bruker Avance II spectrometer (Bruker Biospin, Coventry, UK) with a 4 mm HR-MAS probe at 310 K while spinning at 5 kHz. 1H NMR spectra were acquired using a Carr-Purcell Meiboom-Gill pre-saturation (cpmgpr1d) pulse sequence with a spectrum width of 16.0 ppm and 9615 data points using 64 transients. Free induction decay was multiplied by an exponential function with 0.293 Hz line broadening. 2D ¹H-¹H correlation spectroscopy (COSY) and ¹H-¹³C Heteronuclear Single Quantum Correlation (HSQC) experiments were performed on representative data sets using standard Bruker settings. 2,7-dichlorodihydrofluorescein diacetate (DCFH-DA)fluorescence—EMRSA-15 and P. aeruginosa RP73 were cultured overnight at 37° C. in either MHB or RPMI (5% FBS) in the presence and absence of D-pleurocidin or its analogues. Samples were pelleted by centrifugation and diluted to an OD₆₀₀ of 0.6 in phosphate buffered saline (PBS). The cell suspension was incubated in darkness for 1 hour at 37° C. with 5 μM DCFH-DA. Upon completion of incubation, the cell suspension was washed with PBS to remove excess dye and incubated in darkness at 37° C. for 5 minutes to equilibrate. Fluorescence spectra (515-545 nm) were obtained using a Varian Cary Eclipse fluorescence spectrometer at 37° C. with excitation at 485 nm.

Liquid state NMR—¹H NMR spectra were acquired under automation at 298 K and 700 MHz on a Bruker Avance II 700 NMR spectrometer (Bruker Biospin, Coventry, UK) equipped with a 5 mm QCl helium-cooled cryoprobe and a cooled SampleJet sample changer. 1D CPMG-presat (cpmgpr1d) experiments were acquired with 32 transients, a spectral width of 19.8 ppm and 11,904 data points. 2D ¹H-¹H correlation spectroscopy (COSY) and ¹H-¹³C Heteronuclear Single Quantum Correlation (HSQC) experiments were performed on representative data sets using standard Bruker settings.

Data processing—The spectra was Fourier transformed automatically using standard Bruker commands and manually phased and baseline corrected in Bruker TopSpin 4.0 (Bruker Biospin, Coventry, UK). Spectral pre-processing, cross-validation and multivariate analysis were carried out using the in-house program developed by Dr Louic Vermeer and MVAPACK, an opensource Octave library for NMR metabolomic data processing and analysis.⁵⁷ Pre-processing modifies NMR spectra and reduces the variances and influences which are not of interest and may interfere with data analysis, for example, residual water peak or noise and the TSP reference peak. Initially a Principle Component Analysis (PCA) was carried out to identify clustering spectra and potential outliers in the data due to e.g. poor baseline. Data were then analysed using the probabilistic quotient normalization (PQN) and auto scaled before binning and alignment. Volcano plots were made using the binned data for every control sample (no AMP or tobramycin) versus challenge sample (with an AMP or tobramycin), comparing fold changes in metabolites, defined as the ratio between the control and the challenge. Box plots were generated using changes in normalised intensity for each metabolite and significant differences between challenges and controls were determined using a one way-ANOVA. Volcano plots and box plots were generated using in house software developed for Jupyter Notepad using Python 3.7.0. Metabolites were assigned using the databanks: Chenomx NMR suite software (Chenomx Inc., Canada), Human Metabolome Database (HMDB), Biological Magnetic Resonance Data Bank (BMRB), E. coli Metabolome Database (ECMDB) and AOCS lipid library; and a comparison of chemical shifts to the literature, which was confirmed using 2D NMR spectra. Annotated spectra can be found in the supplementary materials. Metaboanalyst and KEGG were later used to identify key pathways that may be affected by these metabolites.

Murine EMRSA-15 lung infection model. In one embodiment this was carried out using the method described previously,⁵⁸ an overnight culture of EMRSA-15 was prepared in tryptic soy broth (TSB). EMRSA-15 was cultured in TSB overnight at 37° C., adjusted to a starting OD₆₀₀ of 0.025, and grown for additional 4 h for agar beads preparation. EMRSA-15 was embedded into agar beads by mixing the overnight culture with molten tryptic soy agar, which was then spun into warmed mineral oil. The preparation was cooled and centrifuged at 2,700 g, the remaining oil was eliminated and the beads were washed in sterile PBS. The colony forming unit (cfu) content of the beads slurry was subsequently quantified on TSA plates and the beads slurry then diluted to either 2×10⁷ cfu/ml in sterile PBS to deliver a final dose of either 1×10⁶ cfu/mouse. Animal work was performed in accordance with the Animals (Scientific Procedures) Act of 1986 (United Kingdom). On day 0, male C57B16J mice (8-10 weeks, Charles River) were anesthetized under Isoflurane and 50 μl inoculum of EMRSA-15 embedded in agar beads (10⁶ cfu/mouse) was instilled directly into the respiratory tract via oropharyngeal dosing (o.a) to reduce severity associated with surgery. Sham control mice were inoculated with sterile PBS agar beads. Animals were treated with either Vehicle (Saline), 200 mg/kg vancomycin or 0.05, 0.5 or 5 mg/kg D-pleurocidin-KR at 4, 24 and 30 hours post infection via intravenous injection, reaching a total dosage of 600 mg/kg vancomycin or 0.15, 1.5 or 15 mg/kg D-pleurocidin-KR. Body weight was measured daily, and animals monitored at regular intervals for signs of pain and distress. 48 hours post pulmonary infection, animals were terminally euthanized using 25% Urethane via intra-peritoneal injection. Lungs, were removed and homogenized in 2 ml PBS. Samples were serially diluted 1:10 in PBS and plated for CFU count. Lung homogenates were then centrifuged at 14000 rpm for 30 min at 4° C. and the supernatants were stored at −80° C. for future analysis. For bronchoalveolar lavage fluid collection and analysis (total and differential cell count), a 22-gauge catheter was inserted into the trachea and BAL was recovered by instillation of 0.5 ml of sterile PBS three times and total cells counts were performed by adding Turks stain in a 1:1 ratio. Total cells were then enumerated on an improved Naubauer haemocytometer. BAL fluid was then centrifuged, and supernatants were stored at −20° C. Cytospin slides were prepared and 100 μl of BAL was added into a cytospin funnel and centrifuged at 1000 rpm for 1 minute on a medium acceleration. The slides were then stained using Diff-Quick (Dade, Biomap, Italy).

More suitably Murine EMRSA-15 lung infection model was carried out using the method described previously,⁵⁸ an overnight culture of EMRSA-15 was prepared in tryptic soy broth (TSB). EMRSA-15 was cultured in TSB overnight at 37° C., adjusted to a starting OD₆₀₀ of 0.025, and grown for additional 4 h for agar beads preparation. EMRSA-15 was embedded into agar beads by mixing the overnight culture with molten tryptic soy agar, which was then spun into warmed mineral oil. The preparation was cooled and centrifuged at 2,700 g, the remaining oil was eliminated, and the beads were washed in sterile PBS. The colony forming unit (CFU) content of the beads slurry was subsequently quantified on TSA plates and the beads slurry then diluted to either 2×10⁷ CFU/ml in sterile PBS to deliver a final dose of either 1×10⁶ CFU/mouse. Animal work was performed in accordance with the Animals (Scientific Procedures) Act of 1986 (United Kingdom). On day 0, male C57B16J mice (8-10 weeks, Charles River) were anesthetized under Isoflurane and 50 μl inoculum of EMRSA-15 embedded in agar beads (10⁶ CFU/mouse) was instilled directly into the respiratory tract via oropharyngeal dosing (o.a.) to reduce severity associated with surgery. Sham control mice were inoculated with sterile PBS agar beads. Animals were treated with either Vehicle (Saline), 200 mg/kg vancomycin or 0.05, 0.5 or 5 mg/kg D-pleurocidin-KR at 4, 24 and 30 hours post infection via intravenous injection, reaching a total dosage of 600 mg/kg vancomycin or 0.15, 1.5 or 15 mg/kg D-pleurocidin-KR. Body weight was measured daily, and animals monitored at regular intervals for signs of pain and distress. 48 hours post pulmonary infection, animals were terminally euthanized using 25% Urethane via intra-peritoneal injection. CFUs were quantified in lung homogenates. Lung homogenates were then centrifuged at 14000 rpm for 30 min at 4° C. and the supernatants were stored at −80° C. for future analysis. For BAL fluid collection and analysis (total and differential cell count), a 22-gauge catheter was inserted into the trachea and BAL was recovered by instillation of 0.5 ml of sterile PBS three times and total cells counts were performed by adding Turks stain in a 1:1 ratio. BAL fluid was then centrifuged, and supernatants were stored at −20° C. Total levels of TNFα, IL-6, KC and MCP-1 were quantified from BAL fluid collected from all animals and measured by ELISA according to the manufacturer protocol (R&D Systems UK). Concentrations of all mediators were then normalised to total protein content of BAL fluid quantified via BCA (Bicinchoninic Acid) total protein assays per-formed to manufacturers protocols (Thermofisher Scientific). Data presented are an aggregate of two independent repeats, each with control groups (n=6) and peptide treatment groups (n=4). Statistical analysis was by One-way ANOVA with Dunnett's multiple comparisons test.

### Acknowledgments

NMR experiments described in this paper were produced using the facilities of the Centre for Biomolecular Spectroscopy, King's College London. HR-MAS NMR experiments performed at the Francis Crick Institute. This work used the ARCHER UK National Supercomputing Service (http://www.archer.ac.uk).

### Additional Information

Supplementary Information including more extensive analysis of the MD simulation data, Circular Dichroism experiments and both liquid and HR-MAS NMR metabolomic data are available. Structural coordinates are deposited in the Protein Data Bank (www.rcsb.org) and Biological Magnetic Resonance Bank (BMRB; www.bmrb.wisc.edu) under accession codes of 6RSF and 6RSG (PDB) and 34404 and 34405 (BRMB) for pleurocidin-KR and pleurocidin-VA respectively. In addition to the structural coordinates the datasets generated during and/or analysed during the current study are available.

### REFERENCES

(1) O'Neill, J. (2016). Tackling drug-resistant infections globally: final report and recommendations. 1st ed. [ebook] Available at: https://amr-review.org/sites/default/files/160518_Final %20paper_with %20cover.pdf [Accessed 18 May 2017].
(2) Czaplewski, L. et al (2016) Alternatives to antibiotics—a pipeline portfolio review. Lancet. Infect. Dis. 16, 239-251.
(3) Hale, J. D. F. & Hancock, R. E. W. (2007) Alternative mechanisms of action of cationic antimicrobial peptides on bacteria. Expert Rev. Anti Infect. Ther. 5, 951-959.
(4) Hancock, R. E. W. & Sahl, H-G. (2006) Antimicrobial and host-defense peptides as new anti-infective therapeutic strategies. Nature Biotech. 24, 1551-1557 (2006).
(5) Cole, A. M., Weis, P. & Diamond, G. (1997) Isolation and characterization of pleurocidin, an antimicrobial peptide in the skin secretions of Winter Flounder. J. Biol. Chem. 272, 12008-12013.
(6) Saint, N., Cadiou, H., Bessin, Y. & Molle, G. (2002) Antibacterial peptide pleurocidin forms ion channels in planar lipid bilayers. Biochim. Biophys. Acta 1564, 359-364 (2002).
(7) Yoshida, K., Mukai, Y., Niidome, T., Takashi, C., Tokunaga, Y., Hatakeyama, T. & Aoyagi, H. (2001) Interaction of pleurocidin and its analogs with phospholipid membrane and their antibacterial activity. J. Peptide Res. 57, 119-126.
(8) Brogden, K. A. (2005) Antimicrobial peptides: pore formers or metabolic inhibitors in bacteria?Nat. Rev. Microbiol. 3, 238-250.
(9) Kozlowska, J., et al. (2014) Combined systems approaches reveal highly plastic responses to antimicrobial peptide challenge in Escherichia coli. PLoS Pathogens 10, e1004104.
(10) Patrzykat, A., Friedrich, C. L., Zhang, L., Mendoza, V. & Hancock, R. E. W. (2002) Sublethal concentrations of Pleurocidin-derived antimicrobial peptides inhibit macromolecular synthesis in Escherichia coli. Antimicrob. Agents Chemother. 46, 605-614.
(11) Lan, Y. et al. (2010) Structural contributions to the intracellular targeting strategy of antimicrobial peptides. Biochim. Biophys. Acta 1798, 1934-1943.
(12) Amos, S-B. T. A., Vermeer, L. S., Kozlowska, J., Davy, M., Ferguson, P., Bui, T. T., Drake, A. F., Lorenz, C. D. & Mason, A. J. (2016) Antimicrobial peptide potency is facilitated by greater conformational flexibility when binding to Gram-negative bacterial inner membranes. Scientific Reports 6, 37639
(13) Lim, S. S et al. (2004) Effects of two glycine residues in positions 13 and 17 of pleurocidin on structure and bacterial cell selectivity. Protein Pept. Lett. 11, 35-40.
(14) Epand, R. M. & Epand, R. F. (2011) Bacterial membrane lipids in the action of antimicrobial agents. J. Pept. Sci. 17, 298-305.
(15) Sohlenkamp, C. & Geiger, O. (2016) Bacterial membrane lipids: diversity in structures and pathways. FEMS Microbiology Reviews 40, 133-159.
(16) Pezzulo, A. A. et al. (2012) Reduced airway surface pH impairs bacterial killing in the porcine cystic fibrosis lung. Nature 487, 109-113
(17) Shah, V. S. et al. (2016) Airway acidification initiates host defense abnormalities in cystic fibrosis mice. Science 351, 503-507.
(18) Syvitski, R. T., Burton, I., Mattatall, N. R., Douglas, S. E. & Jakeman, D. L. (2005) Structural characterization of the antimicrobial peptide Pleurocidin from Winter Flounder. Biochemistry 44, 7282-7293.
(19) Young, Y. J., Shin, S. Y., Lim, S. S., Hahm. K-S. & Kim, Y. (2006) Structure and bacterial cell selectivity of a fish-derived antimicrobial peptide, pleurocidin. J. Microbiol. Biotechnol. 16, 880-888.
(20) Manzo, G. et al (2019) Temporin L and aurein 2.5 have identical conformations but subtly distinct membrane and antibacterial activities. Scientific Reports DOI: 10.1038/s41598-019-47327-w
(21) Touw, W. G., et al (2015) A series of PDB related databases for everyday needs. Nucleic Acids Research 43 (Database issue), D364-D368.
(22) Nagy, G. & Oostenbrink, C. (2014) Dihedral-based segment identification and classification of biopolymers I: proteins. J. Chem. Inf. Model. 54, 266-277.
(23) Leontiadou, H., Mark, A. E. & Marrink, S. J. (2006) Antimicrobial peptides in action. J. Am. Chem. Soc. 128, 12156-12161.
(24) Sengupta, D., Leontiadou, H., Mark, A. E. & Marrink, S. J. (2008) Toroidal pores formed by antimicrobial peptides show significant disorder. Biochim. Biophys. Acta 1778, 2308-2317.
(25) Wiegand I, Hilpert K, Hancock RE (2008) Agar and broth dilution methods to determine the minimal inhibitory concentration (MIC) of antimicrobial substances. Nature Protocols 3, 163-175.
(26) Lin, L. et al (2015) Azithromycin synergizes with cationic antimicrobial peptides to exert bactericidal and therapeutic activity against highly multidrug-resistant gram-negative bacterial pathogens. EBioMedicine 2, 690-698.
(27) Ersoy, S. C. (2017) Correcting a fundamental flaw in the paradigm for antimicrobial susceptibility testing. EBioMedicine 20, 173-181.
(28) Choi, H. & Lee, D. G. (2012) Antimicrobial peptide pleurocidin synergizes with antibiotics through hydroxyl radical formation and membrane damage, and exerts antibiofilm activity. Biochim. Biophys. Acta 1820, 1831-1838.
(29) Burke, K. A. & Lascelles, J. (1975) Nitrate reductase system in Staphylococcus aureus wild type and mutants. J. Bacteriol. 123, 308-316.
(30) Halsey, C. R. et al. (2017) Amino acid catabolism in Staphylococcus aureus and the function of carbon catabolite repression. mBio 8: e01434-16.
(31) Anderson, J. S., Meadow, P. M., Haskin, M. A. & Strominger, J. L. (1966) Biosynthesis of the peptidoglycan of bacterial cell walls. I. Utilization of uridine diphosphate acetylmuramyl pentapeptide and uridine diphosphate acetylglucosamine for peptidoglycan synthesis by particulate enzymes from Staphylococcus aureus and Micrococcus lysodeikticus. Arch. Biochem. Biophys. 116, 487-515.
(32) Aria-Barrau, E. et al (2004) The homogentisate pathway: a central catabolic pathway involved in the degradation of L-phenylalanine, L-tyrosine, and 3-hydroxyphenylacetate in Pseudomonas putida. J. Bacteriol. 186, 5062-5077.
(33) Anderson, C. B. & Witter, L. D. (1982) Glutamine and proline accumulation by Staphylococcus aureus with reduction in water activity. Appl. Environ. Microbiol. 43, 1501-1503.
(34) Kraus, D., Kalnacher, H., Buschmann, J., Berger-Bächi, B., Götz, F. & Peschel, A. (2007) Muropeptide modification-amidation of peptidoglycan d-glutamate does not affect the proinflammatory activity of Staphylococcus aureus. Infect. Immun. 75, 2084-2087.
(35) Lin, Y-C., Cornell, W. C., Jo, J., Price-Whelan, A. & Dietrich, L. E. P. (2018) The Pseudomonas aeruginosa complement of lactate dehydrogenases enables use of D- and L-lactate and metabolic cross-feeding. mBio 9, e00961-18
(36) Koo, H. B. & Seo, J. (2019) Antimicrobial peptides under clinical investigation. Peptide Science e24122.
(37) van der Does A. M., Bogaards, S. J., Jonk, L., Wulferink, M., Velders, M. P. & Nibbering, P. H. (2010) The human lactoferrin-derived peptide hLF1-11 primes monocytes for an enhanced TLR-mediated immune response. Biometals 23, 493-505.
(38) van der Does A. M., Bogaards, S. J., Ravensbergen, B., Beekhuizen, H., van Dissel, J. T. & Nibbering, P. H. (2010) Antimicrobial peptide hLF1-11 directs granulocyte-macrophage colony-stimulating factor-driven monocyte differentiation toward macrophages with enhanced recognition and clearance of pathogens. Antimicrob. Agents Chemother. 54, 811-816.
(39) van der Does, A. M. et al (2012) The human lactoferrin-derived peptide hLF1-11 exerts immunomodulatory effects by specific inhibition of myeloperoxidase activity. J. Immunol. 188, 5012-5019.
(40) Haney, E. F., Straus, S. K. & Hancock, R. E. W. Reassessing the host defence peptide landscape. Front. Chem. 7, 43.
(41) Sieeradzki, K. & Tomasz, A. (1999) Gradual alterations in cell wall structure and metabolism in vancomycin-resistant mutants of Staphylococcus aureus. J. Bacteriol. 181, 7566-7570.
(42) Yu, G., Baeder, D. Y., Regoes, R. R. & Rolff, J. (2016) Combination effects of antimicrobial peptides. Antimicrob. Agents Chemother. 60, 1717-1724
(43) Balasubramanian, D., Harper, L., Shopsin, B. & Torres, V. J. (2017) Staphylococcus aureus pathogenesis in diverse host environments, Pathogens and Disease 75 ftx005
(44) Ibberson, C. B. & Whiteley, M. (2019) The Staphylococcus aureus transcriptome during Cystic Fibrosis lung Infection. mBio 10, e02774-19.
(45) Stokes, J. M., Lopatkin, A. J, Lobritz, M. A. & Collins, J. J (2019) Bacterial metabolism and antibiotic efficacy. Cell Metabolism https://doi.org/10.1016/j.cmet.2010.06.009
(46) Cornforth, D. M. et al. (2018) Pseudomonas aeruginosa transcriptome during human infection. Proc. Natl. Acad. Sci. USA. 115, E5125-E5134.
(47) Bowdish, D. M. E., Davidson, D. J., Scott, M. G. & Hancock, R. E. W. (2005) Immunomodulatory activities of small host defense peptides. Antimicrob. Agents. Chemother. 49, 1727-1732.
(48) Haney, E. F. & Hancock R. E. W. (2013) Peptide design for antimicrobial and immunomodulatory applications. Biopolymers 100, 572-583.
(49) Silva, O. N. et al. (2016) An anti-infective synthetic peptide with dual antimicrobial and immunomodulatory activities. Sci. Rep. 6, 35465.
(50) Keller, R. (2004) The Computer Aided Resonance Assignment Tutorial. Zurich: Cantina Verlag.
(51) Dynamo software: The NMR Molecular Dynamics and Analysis System. Available online at: http://spin.niddk.nih.gov/NMRPipe/dynamo.
(52) Manzo, G. et al. (2016) The singular behavior of a 3-type semi-synthetic two branched polypeptide: three-dimensional structure and mode of action. Phys. Chem. Chem. Phys. 18, 30998-31011.
(53) Abraham, M. J. et al. (2015) GROMACS: High performance molecular simulations through multi-level parallelism from laptops to supercomputers. SoftwareX 1, 19-25.
(54) Best, R. B. et al. (2012) Optimization of the additive CHARMM all-atom protein force field targeting improved sampling of the backbone φ, ψ and side-chain X1 and X² dihedral angles. J. Chem. Theory Comput. 8 (9), 3257-3273.
(55) Huang, J., & MacKerell, A. D. (2013) CHARMM36 all-atom additive protein force field: validation based on comparison to NMR data. J. Comput. Chem. 34, 2135-2145.
(56) Lee, J. et al. (2016) CHARMM-GUI input generator for NAMD, GROMACS, AMBER, OpenMM, and CHARMM/OpenMM simulations using the CHARMM36 additive force field. J. Chem. Theory Comput. 12 (1), 405-413.
(57) Worley B, Powers R. 2014. MVAPACK: a complete data handling package for NMR metabolomics. ACS Chem. Biol. 9, 1138-1144.
(58) Amison, R. T. et al (2018) Platelet depletion impairs host defence to pulmonary infection with Pseudomonas aeruginosa in mice. Am. J. Resp. Cell Mol. Biol. 58, 331-340.
(59) Gautier, R., Douguet, D., Antonny, B. & Drin, G. (2008) HELIQUEST: a web server to screen sequences with specific α-helical properties. Bioinformatics. 24, 2101-2102.

TABLE 1
Peptides sequences, biophysical characteristics and concentration of peptide necessary to
start membrane activity in electrophysiology experiments. All peptides were amidated at the C-
terminus. Hydrophobicity (H) and hydrophobic moment assuming a (μH)ₐ or 3-11 (μH)₃₋₁₁ helix
secondary structure were calculated using HeliQuest.⁵⁹

| | | | | | Peptide concentration | |
|---|---|---|---|---|---|---|
| | | | | | (μM) | |
| Peptide | Sequence | H | (μH)ₐ | (μH)₃₋₁₁ | DPhPE/DPhPG | DPhPG |
| Pleurocidin | GWGSFFKKAAHVGKHVGKAALTHYL | 0.421 | 0.309 | 0.340 | 10 | 5 |
| Pleurocidin- | GWGSFFRRAAHVGRHVGRAALTHYL | 0.418 | 0.311 | 0.342 | 10 | 7.5 |
| KR | | | | | | |
| Pleurocidin- | GWGSFFKKAAHAGKHAGKAALTHYL | 0.348 | 0.278 | 0.323 | — | 2.5 |
| VA | | | | | | |

TABLE 2
Antimicrobial activity and cellular toxicity. M-methicillin sensitive; EMR-epidemic methicillin resistant; VS-vancomycin
sensitive; VR-vancomycin resistant. Values are given for peptides tested in Mueller-Hinton broth with values obtained
in RPMI given in parentheses. The Selectivity Index is the EC₅₀ divided by the MIC in the indicated conditions.
Grey or bold values indicate respectively a significant reduction or improvement in potency in RPMI.

| | | Peptide concentration (μg/ml) | | | | | |
|---|---|---|---|---|---|---|---|
| | | | | | D- | | D- |
| | | | D- | Pleurocidin | pleurocidin- | Pleurocidin- | pleurocidin- |
| | Isolate | Pleurocidin | pleurocidin | KR | KR | VA | VA |
| Gram- | Klebsiella | 4-8 (32) | 4 (8-16) | 2-4 (32) | 2 (4) | 4-8 (32) | 8 (32) |
| negative | pneumoniae | | | | | | |
| | NCTC 13368 | | | | | | |
| | Klebsiella | 4 (32) | 4 (4-8) | 2 (32) | 2 (4) | 4-8 (32) | 4 (32) |
| | pneumoniae | | | | | | |
| | M6 | | | | | | |
| | Acinetobacter | 1-2 (4-8) | 1 (2) | 1-2 (8-16) | 2 (2) | 2 (16) | 1 (8-32) |
| | baumannii | | | | | | |
| | AYE | | | | | | |
| | Acinetobacter | 1-2 (8) | 1 (8) | 1 (8) | 2 (4) | 1-2 (32) | 1 (16) |
| | baumannii | | | | | | |
| | ATCC 17978 | | | | | | |
| | Pseudomonas | 64 (>32) | 2 (16) | 4 (>32) | 4 (16) | 16-32 (>32) | 4 (32) |
| | aeruginosa | | | | | | |
| | PA01 | | | | | | |
| | Pseudomonas | 16-32 (>32) | 8 (16-32) | 8 (>32) | 4 (16) | 32 (>32) | 32 (>32) |
| | aeruginosa | | | | | | |
| | NCTC 13437 | | | | | | |
| | Escherichia coli | 1-2 (16) | 1 (2-4) | 1 (16) | 1 (2) | 1 (32) | 1 (8) |
| | NCTC 12923 | | | | | | |
| Gram- | MS | 4 (2) | 2 (0.5) | 2 (4) | 2 (0.25-0.5) | 8 (32) | 4 (8) |
| positive | Staphylococcus | | | | | | |
| | aureus | | | | | | |
| | ATCC 9144 | | | | | | |
| | EMR | 16 (4) | 16 (1) | 4 (4) | 2 (0.5) | 64 (32) | 32 (8) |
| | Staphylococcus | | | | | | |
| | aureus-15 | | | | | | |
| | EMR | 16 (4) | 16 (1) | 4 (8) | 4 (1) | 64-128 (>32) | 64 (32) |
| | Staphylococcus | | | | | | |
| | aureus-16 | | | | | | |
| | VS | 64 (NG) | 32 (NG) | 16 (NG) | 8 (NG) | 64-128 (NG) | 64 (NG) |
| | Enterococcus | | | | | | |
| | faecalis | | | | | | |
| | NCTC 775 | | | | | | |
| | VR | 16-32 (32) | 16 (4) | 4 (16) | 2 (2) | 32-64 (32) | 16 (32-64) |
| | Enterococcus | | | | | | |
| | faecium | | | | | | |
| | NCTC 12204 | | | | | | |
| Toxicity | HEK293 | (87.5 ± 11.1) | (61.4 ± 3.9) | (40.1 ± 4.1) | (36.4 ± 4.3) | (~400) | (217.3 ± 46.5) |
| | HeLa | (58.2 ± 3.9) | (47.1 ± 2.1) | (30.7 ± 2.6) | (25.3 ± 2.2) | (>400) | (198.2 ± 10.2) |
| | A549 | n.d. | (79.3 ± 7.2) | n.d. | (56.5 ± 4.5) | n.d. | (>250) |
| | Calu-3 | n.d. | (41.9 ± 1.8) | n.d. | (39.1 ± 1.8) | n.d. | (>250) |
| | HEK293/ | 5.5 (21.9) | 3.8 (61.4) | 10.0 (10.0) | 18.2 (72.8) | 6.25 (12.5) | 6.8 (27.2) |
| | EMRSA-15 | | | | | | |
| | A549/ | n.d. | 5.0 (79.3) | n.d. | 28.3 (113.0) | n.d. | >7.8 (>31.2) |
| | EMRSA-15 | | | | | | |
| | HEK293/ | n.d. | 9.2 (5.8) | n.d. | 12.8 (4.6) | n.d. | n.d. |
| | P. aeruginosa | | | | | | |
| | RP73 | | | | | | |
| | (tobramycin) | | | | | | |

TABLE 3
D-pleurocidin analogue synergy. The Fractional Inhibitory
Concentration (FIC) is reported for selected binary antibiotic combinations as an
average ± standard error of three independent repeats. Values in bold are considered to
represent synergy (FIC < 1.0).

| | EMRSA-15 | | P. aeruginosa RP73 | |
|---|---|---|---|---|
| | MH | RPMI | MH | RPMI |
| D-pleurocidin/tobramycin | 0.55 ± | 0.83 ± 0.29 | 1.00 ± | 0.92 ± 0.14 |
| | 0.02 | | 0.00 | |
| D-pleurocidin-KR/ | 0.80 ± | 0.78 ± 0.31 | 0.85 ± | 0.71 ± 0.07 |
| tobramycin | 0.46 | | 0.25 | |
| D-pleurocidin/rifampin | 1.00 ± | 1.00 ± 0.00 | 0.85 ± | 0.60 ± 0.04 |
| | 0.00 | | 0.25 | |
| D-pleurocidin-KR/rifampin | 1.00 ± | 1.00 ± 0.00 | 0.83 ± | 0.61 ± 0.12 |
| | 0.00 | | 0.29 | |
| D-pleurocidin/colistin | n.d | n.d | 1.00 ± | 1.00 ± 0.00 |
| | | | 0.00 | |
| D-pleurocidin-KR/colistin | n.d. | n.d. | 0.75 ± | 1.00 ± 0.00 |
| | | | 0.35 | |

### BRIEF DESCRIPTION OF THE DRAWINGS

Embodiments of the present invention will now be described further, with reference to the accompanying drawings, in which:

FIG. 1 shows graphs and plots

FIG. 2 shows diagrams, plots and graphs

FIG. 3 shows plots and graphs

FIG. 4 shows diagrams and plots and bar charts

FIG. 5 shows plots

Figure P1 shows graphs

Figure P2 shows plots

FIG. 6 shows graphs and a table. Parent=Pleurocidin (SEQ ID NO: 1). D-Parent=Pleurocidin (SEQ ID NO: 1)(all D-amino acids). AP1=Analogue Peptide 1—Pleurocidin-KR (PKR) (SEQ ID NO: 2). AP2=Analogue Peptide 2—Pleurocidin-VA (PVA)(SEQ ID NO: 3). D-AP1=D-Analogue Peptide 1-Pleurocidin-KR (PKR) (SEQ ID NO: 2) (all D-amino acids). D-AP2=Analogue Peptide 2—Pleurocidin-VA (PVA)(SEQ ID NO: 3) (all D-amino acids).

FIG. 7 shows a diagram, bar charts and plots. Peptides as for FIG. 6.

FIG. 8 shows plots. Peptides as for FIG. 6.

FIG. 9 shows a combined computational and experimental biophysics approach has yielded a promising AMP therapeutic. Peptides as for FIG. 6.

FIG. 10 shows plots.

FIG. 11 shows plots.

FIG. 12 shows graphs.

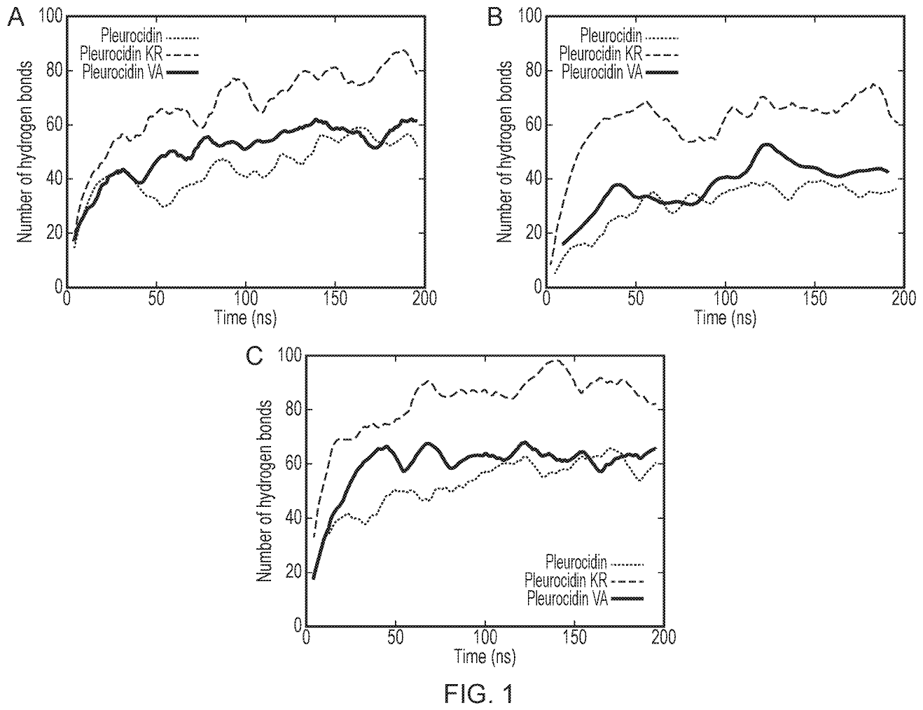

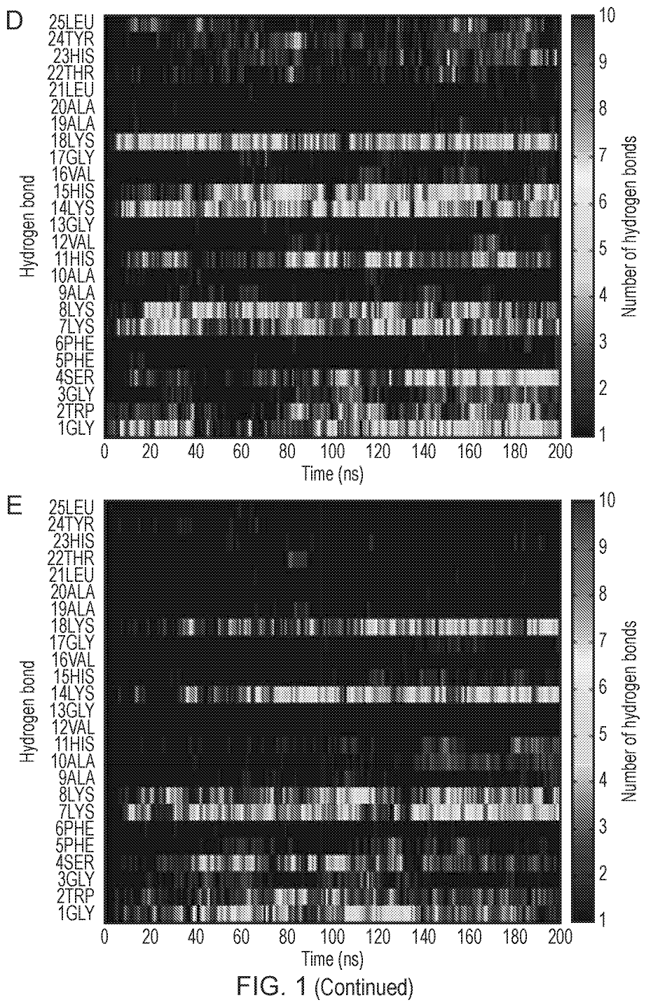

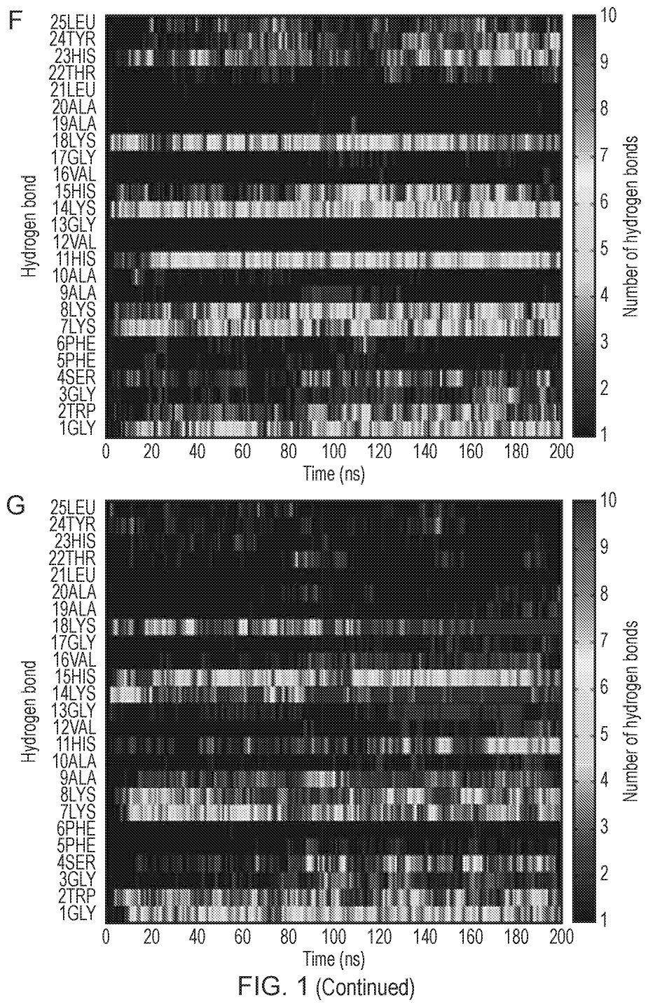

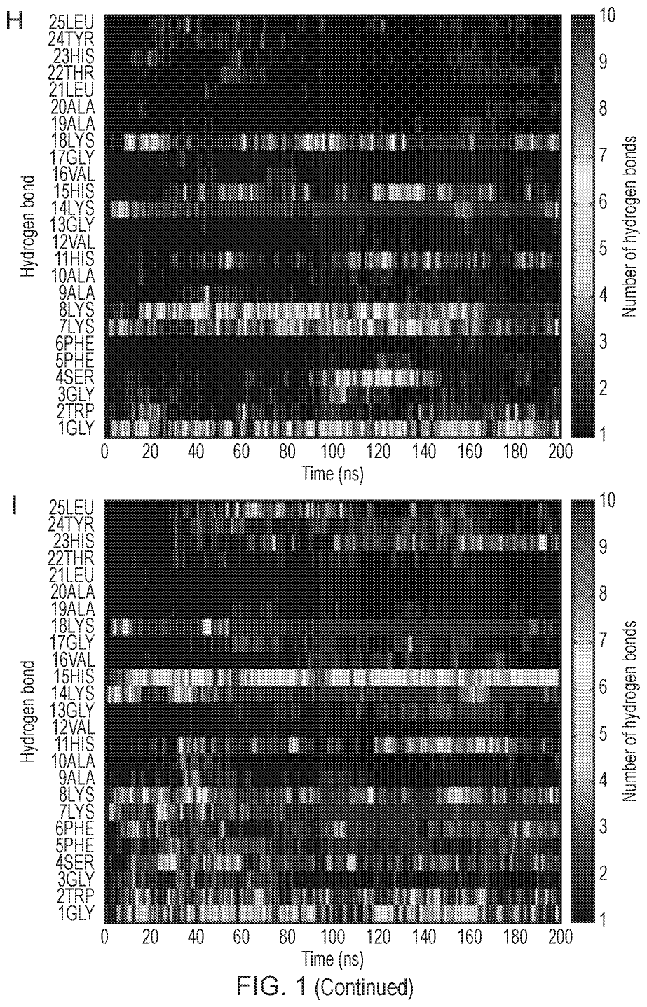

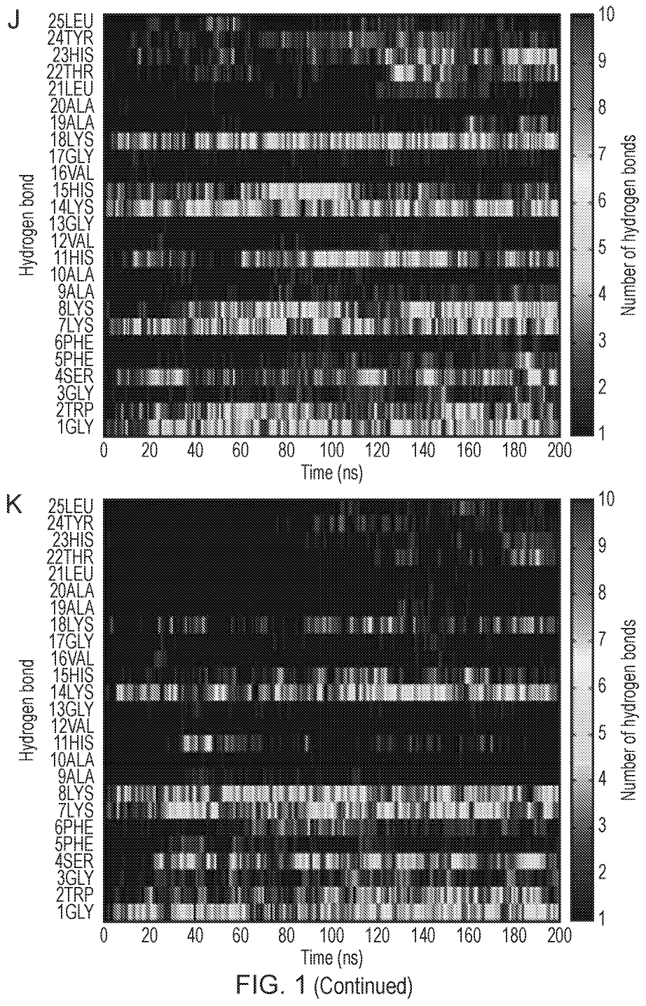

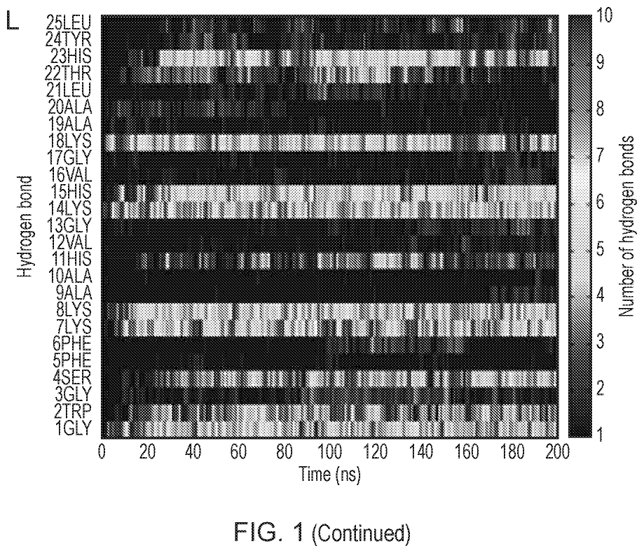

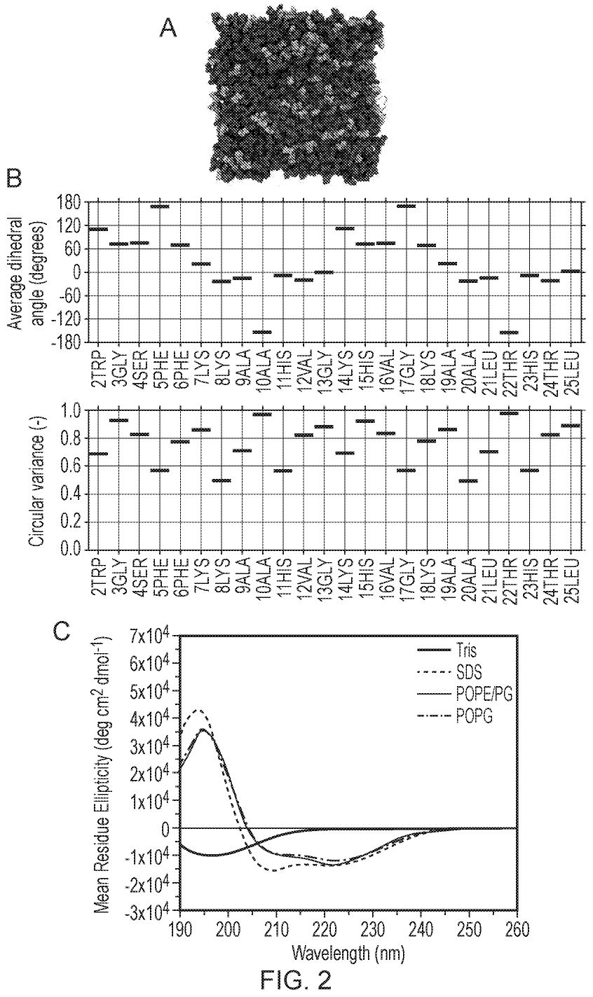

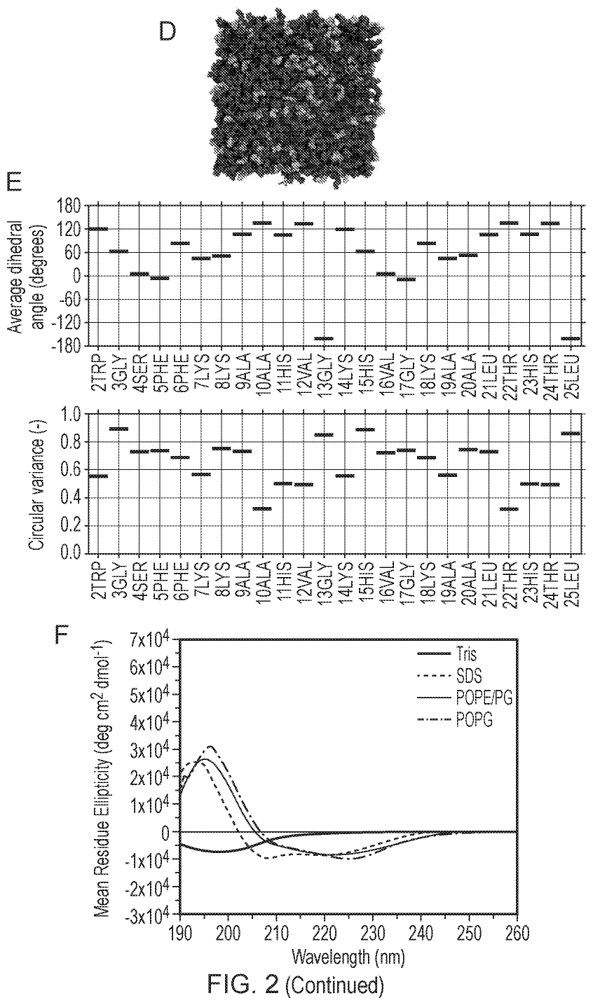

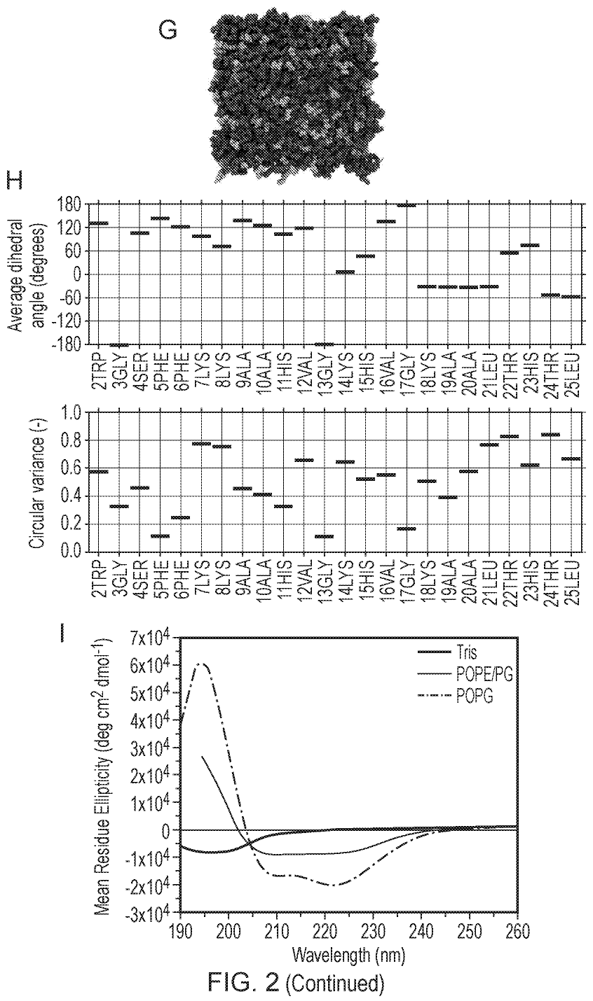

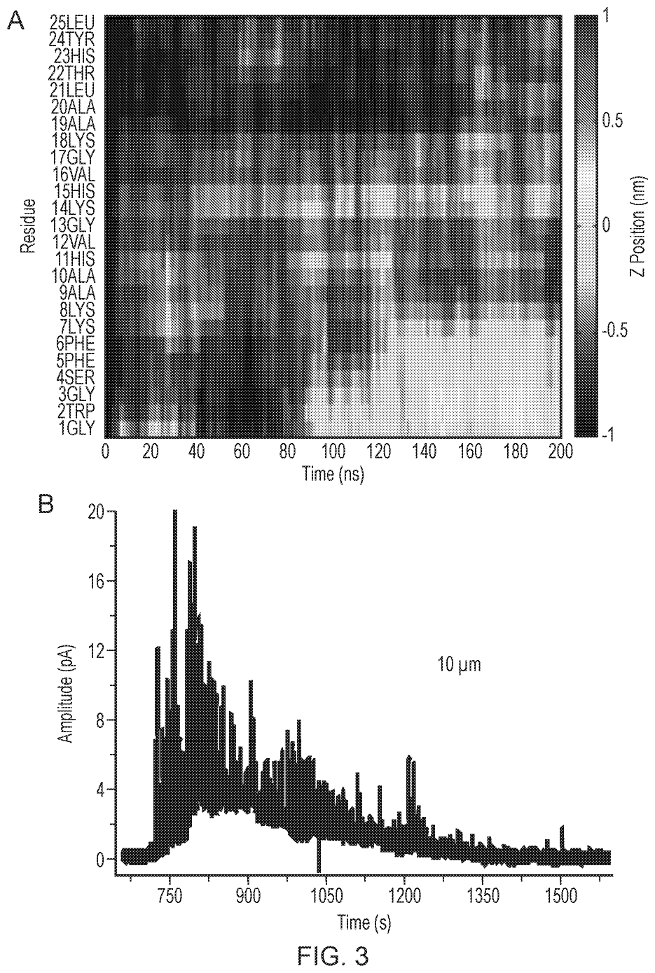

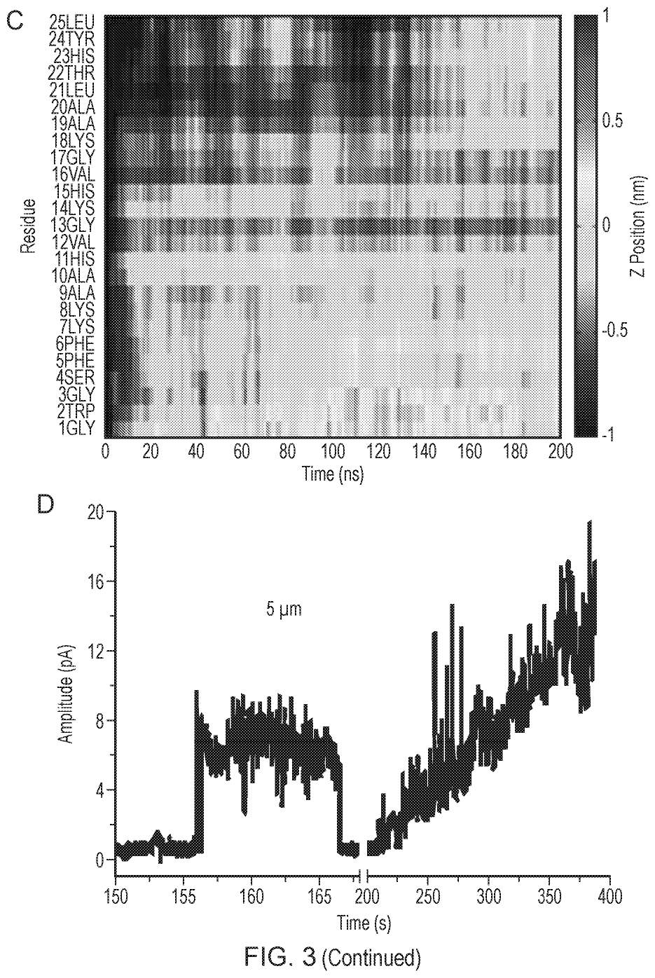

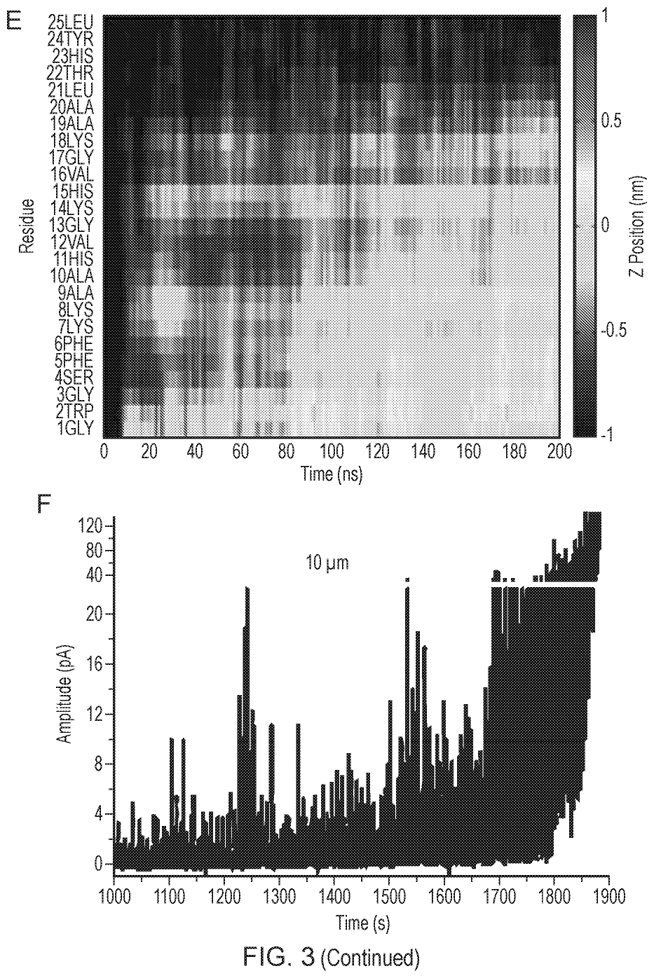

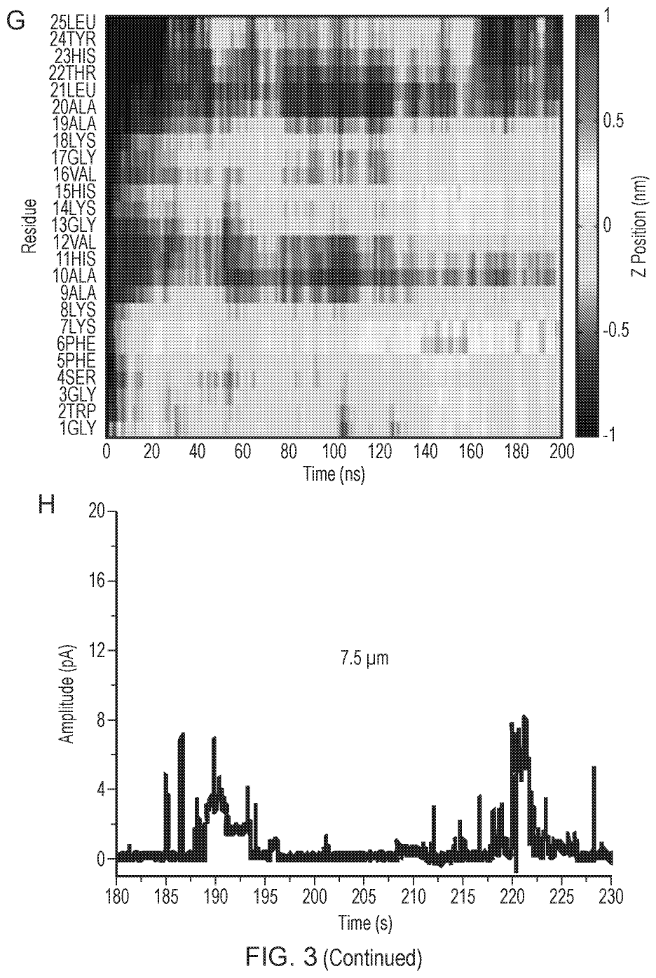

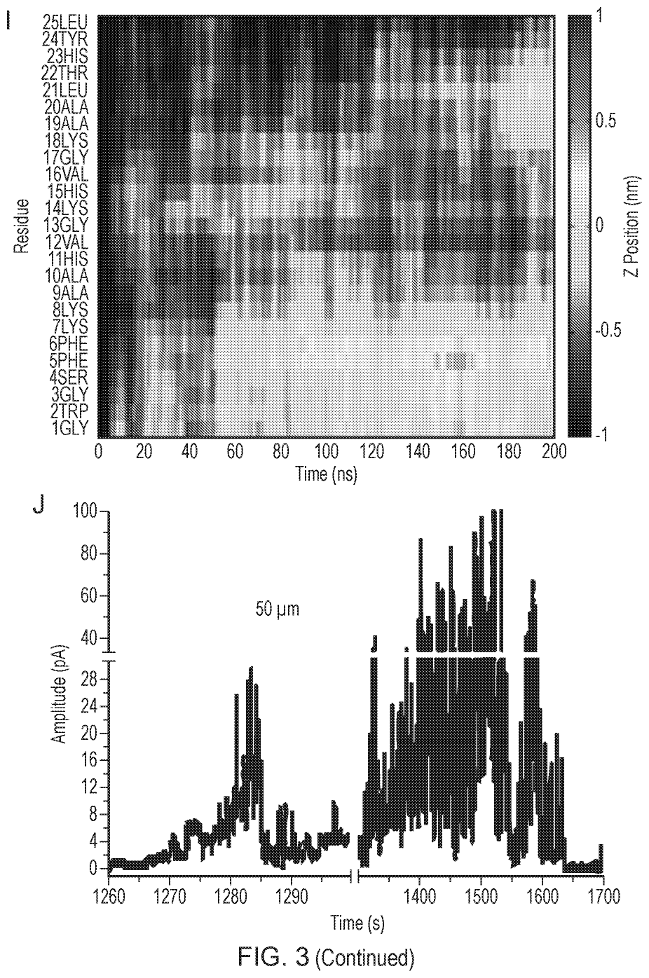

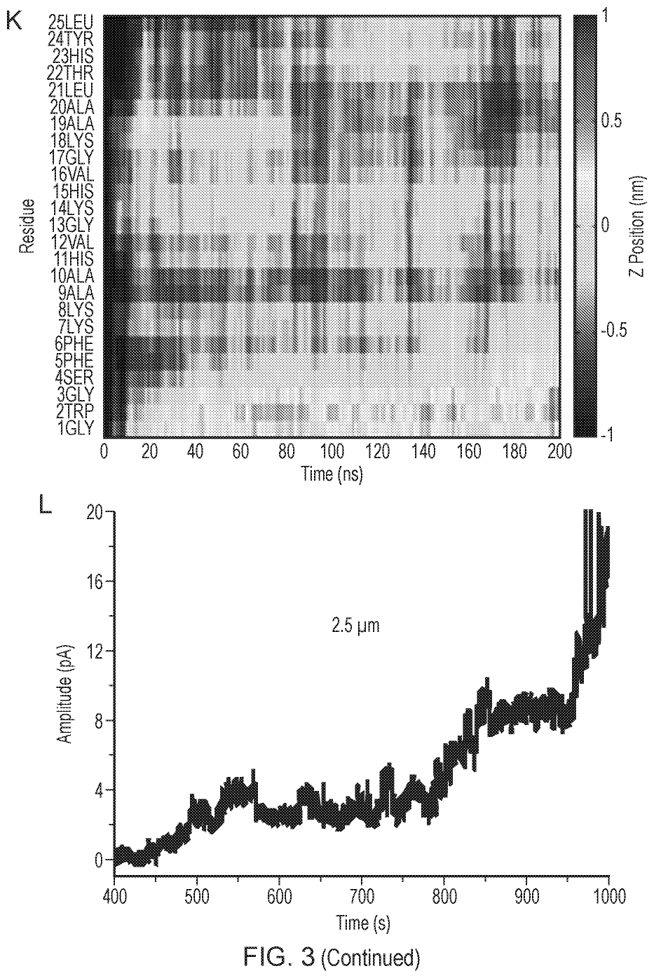

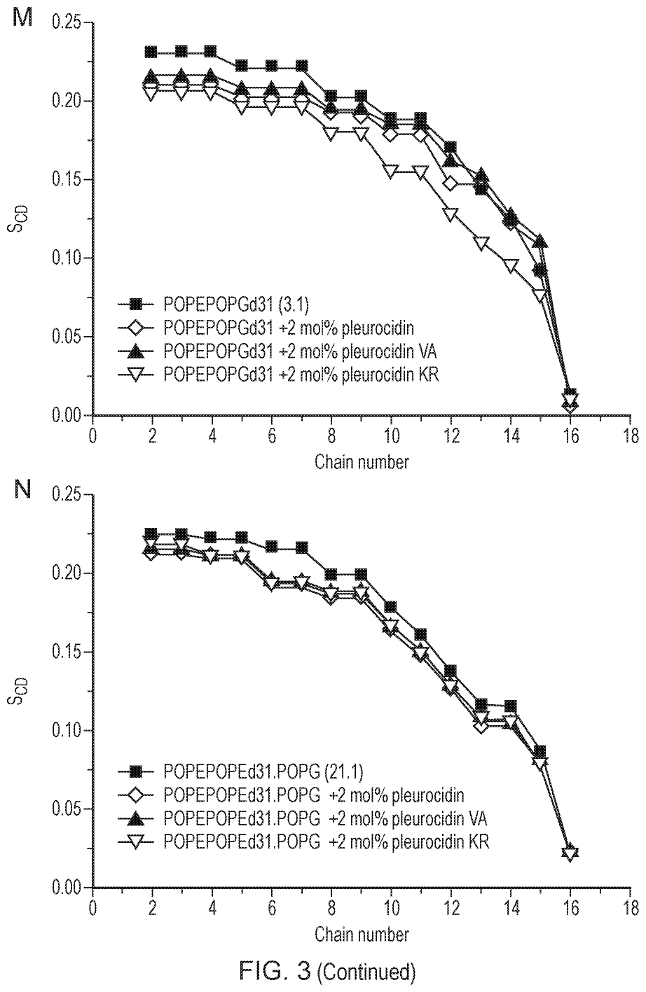

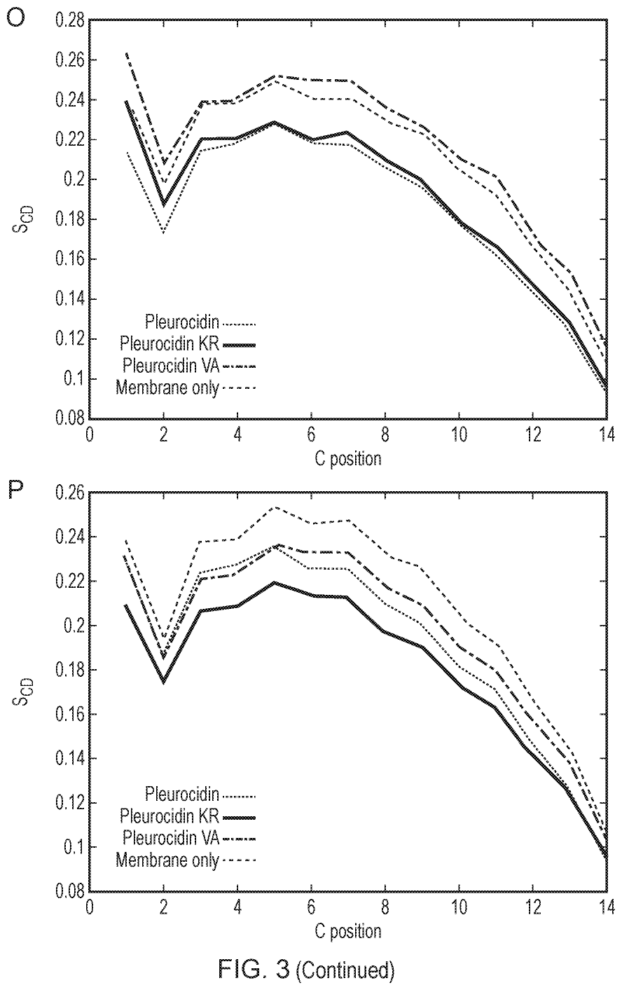

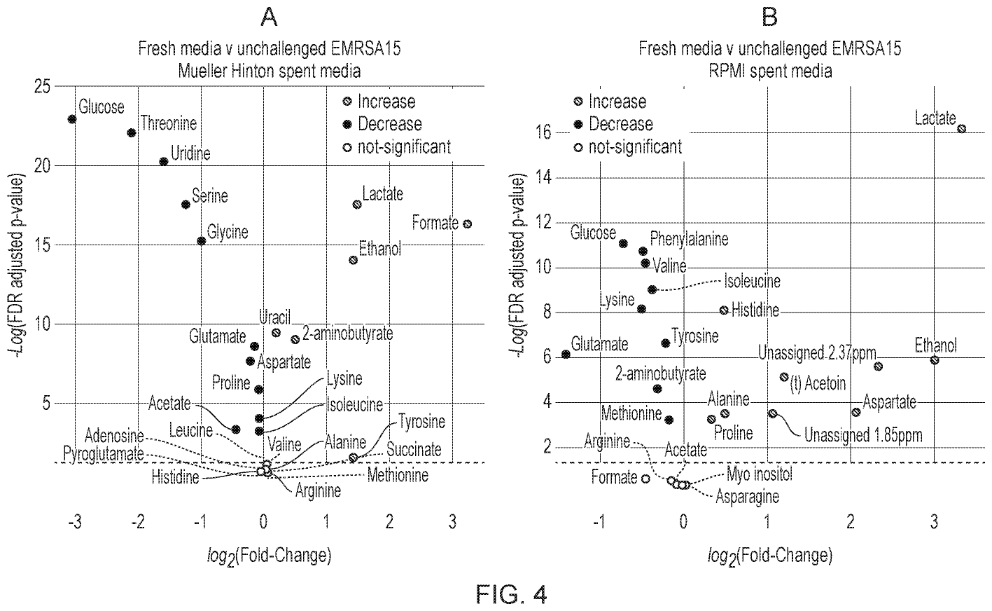

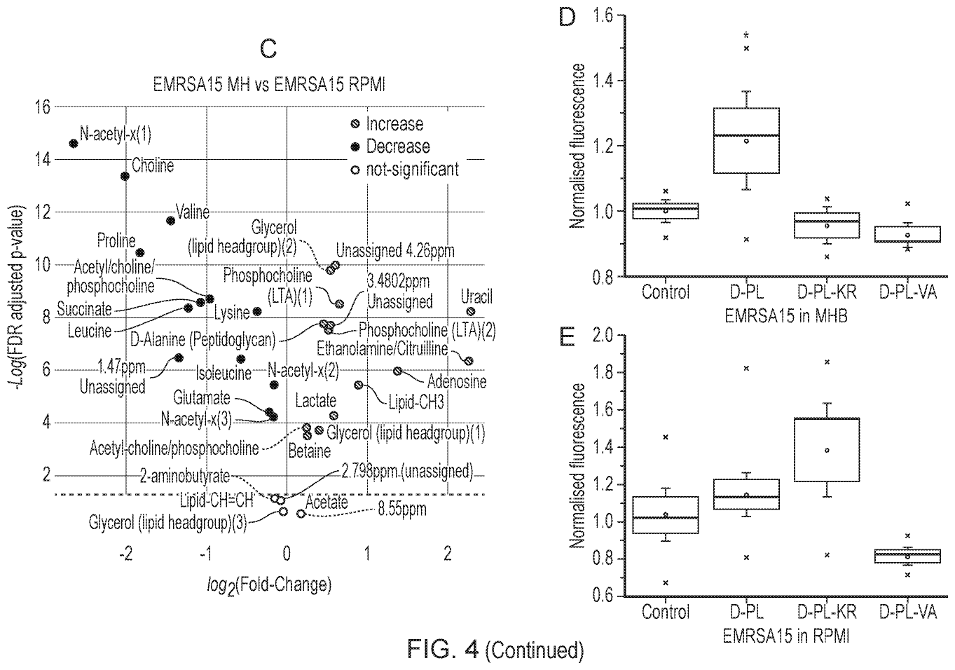

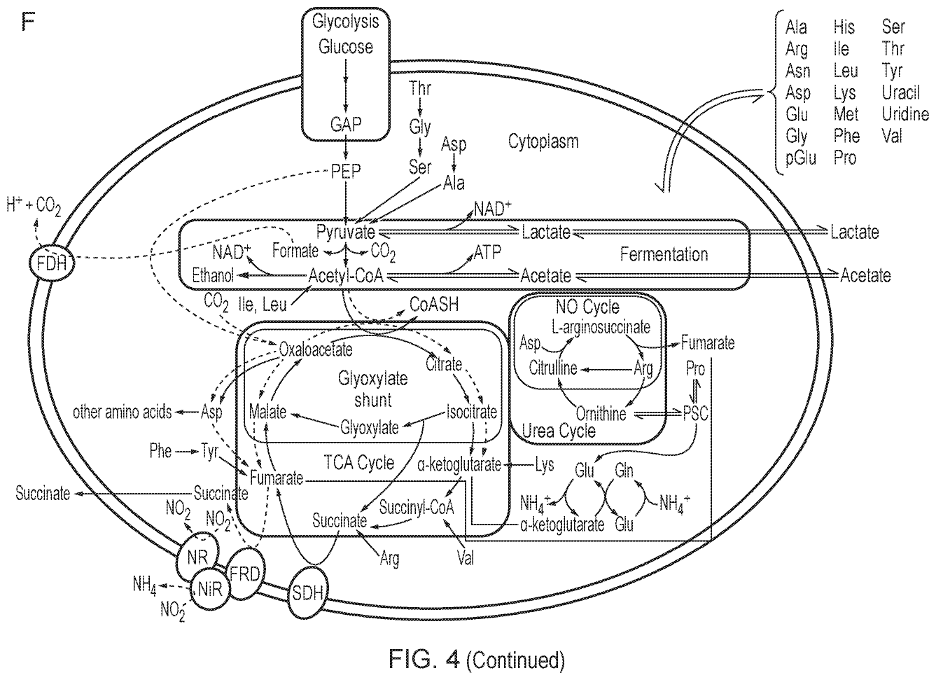

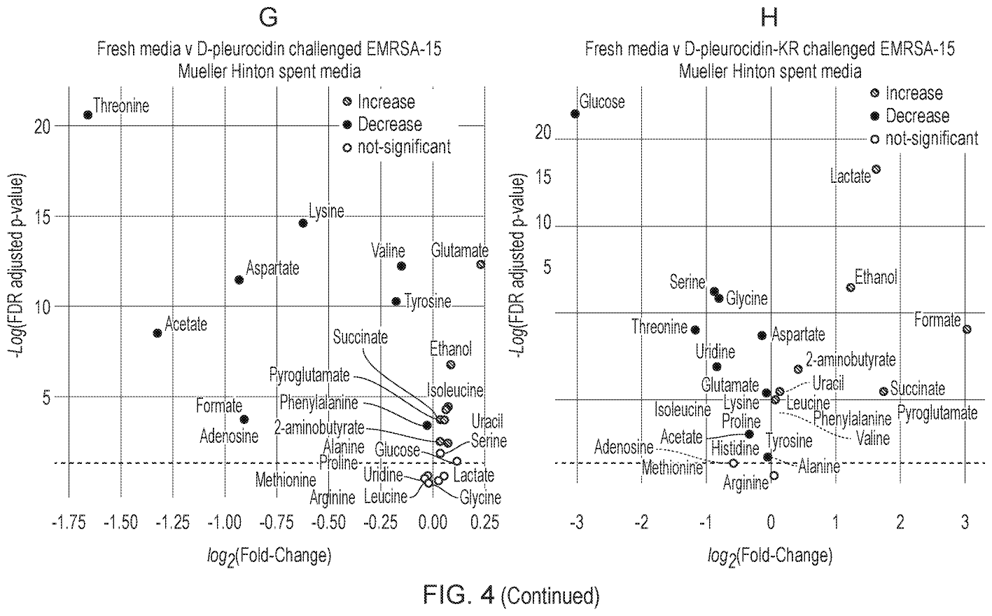

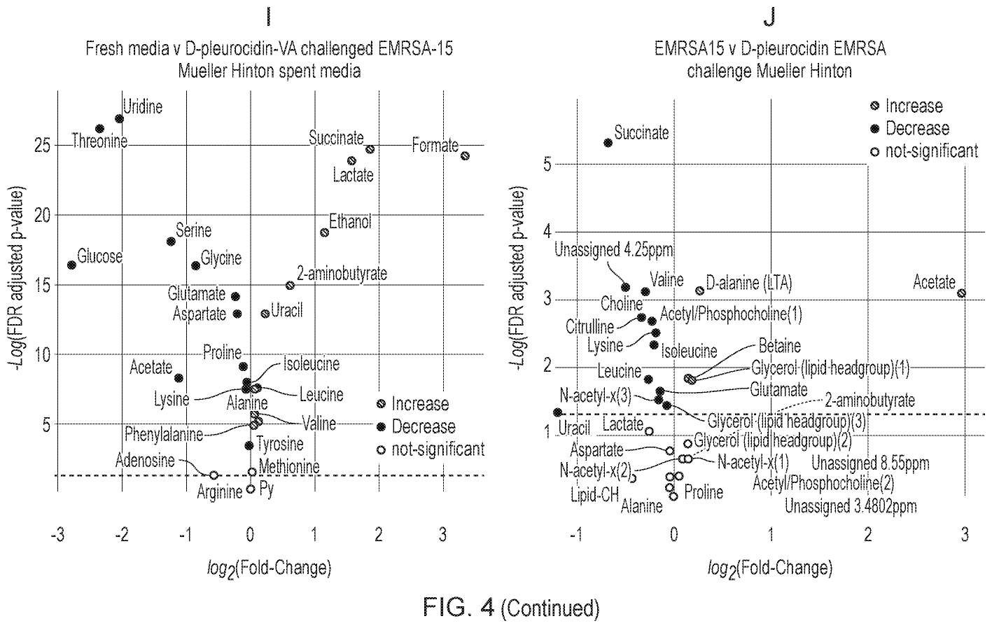

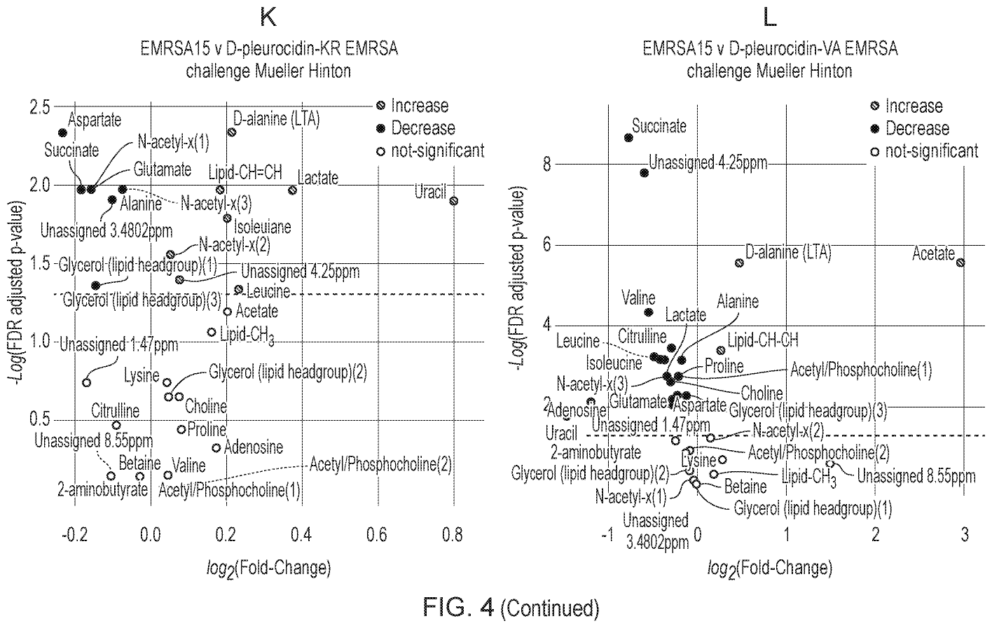

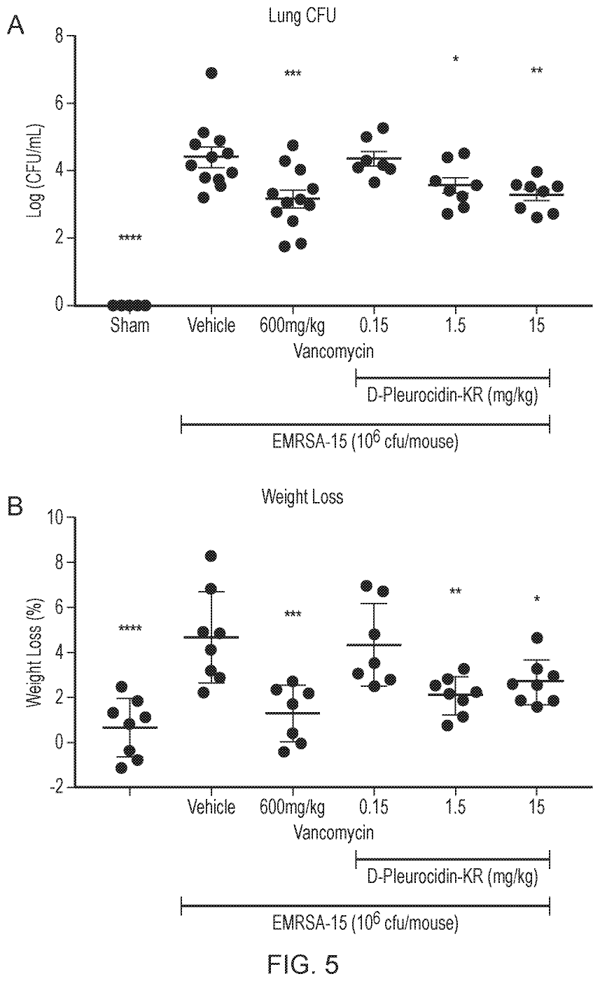

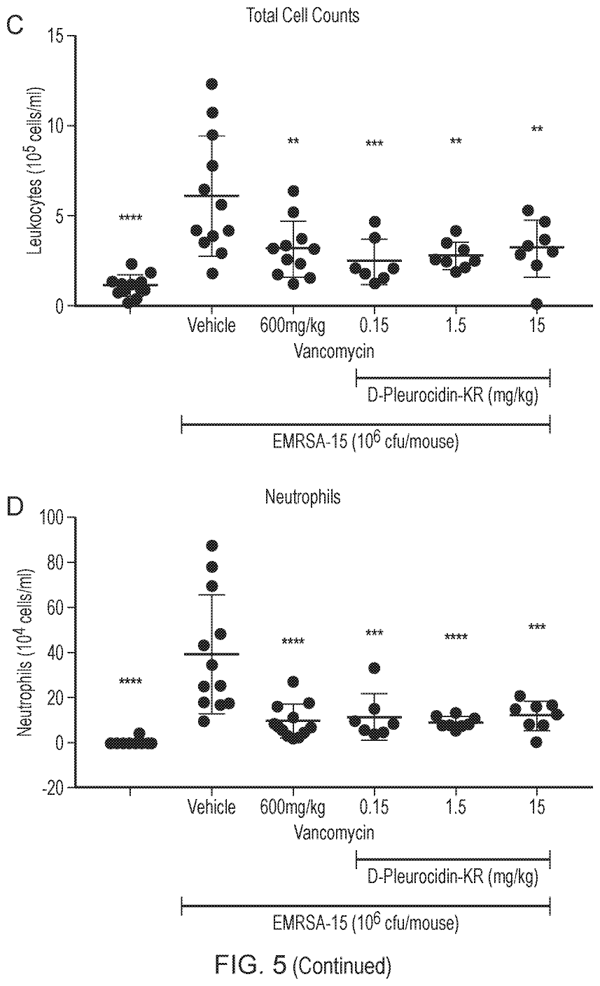

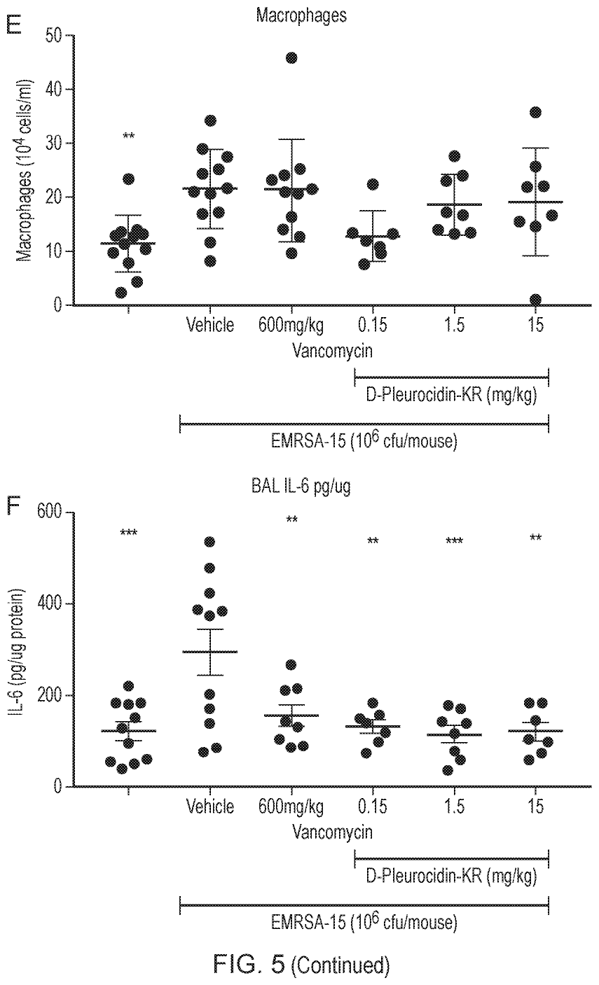

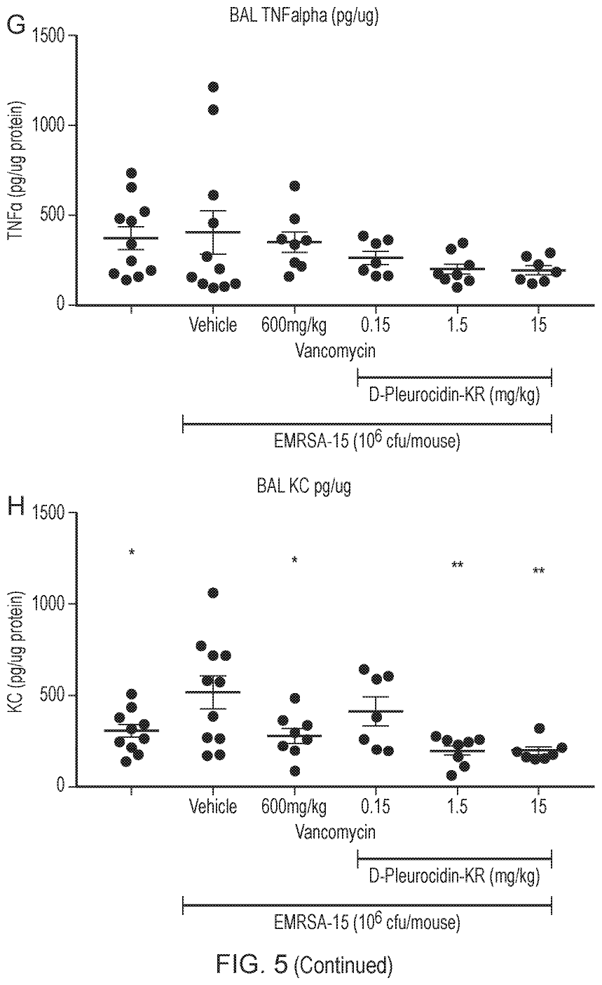

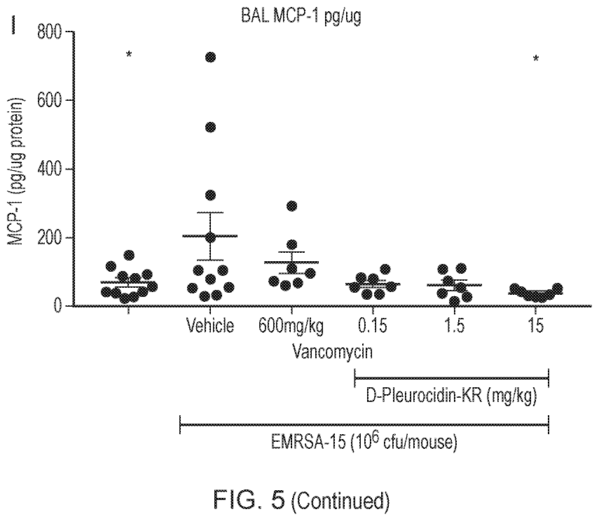

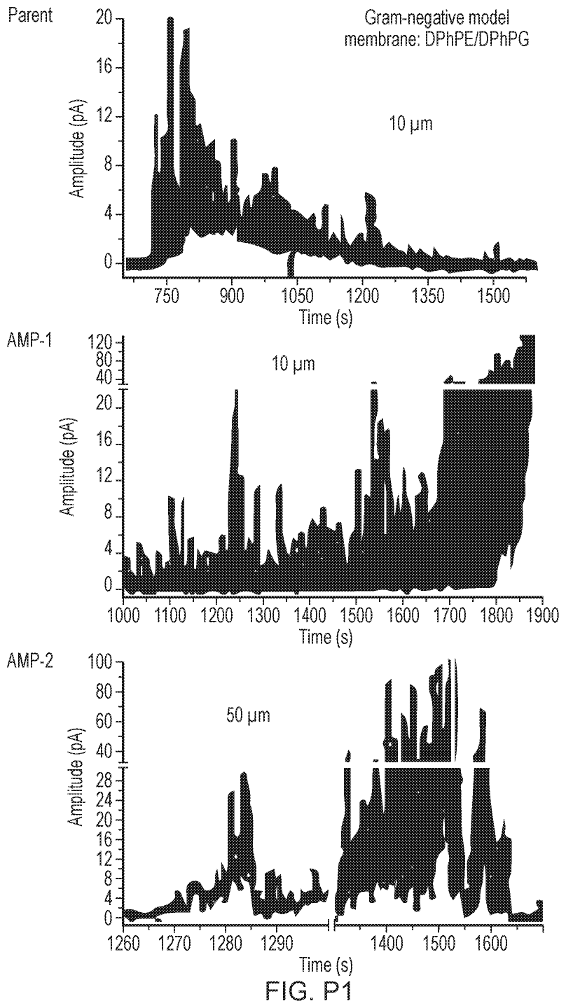

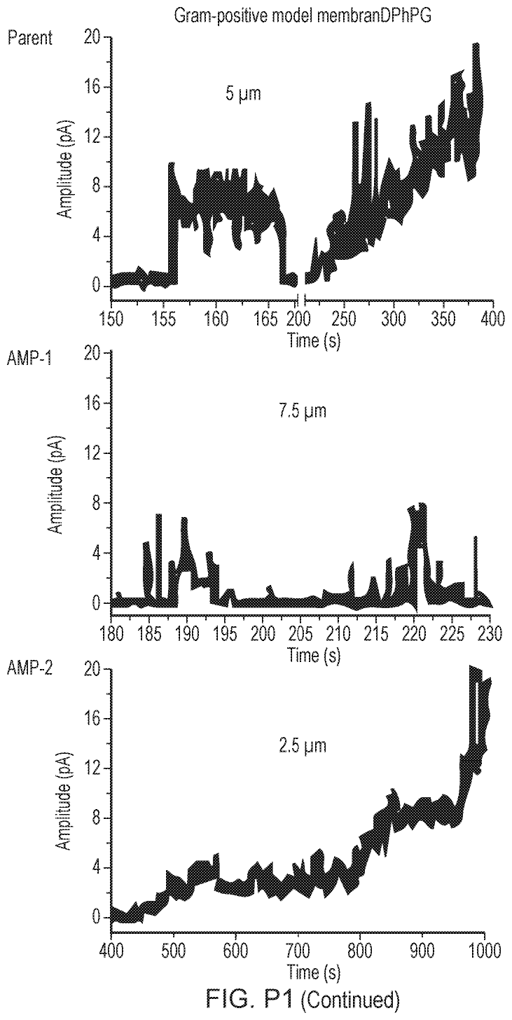

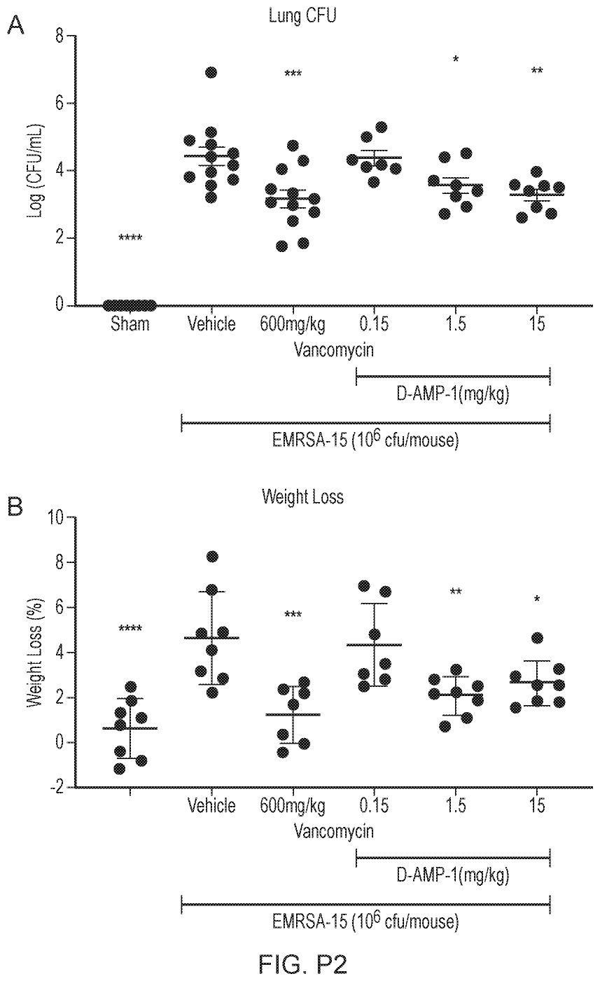

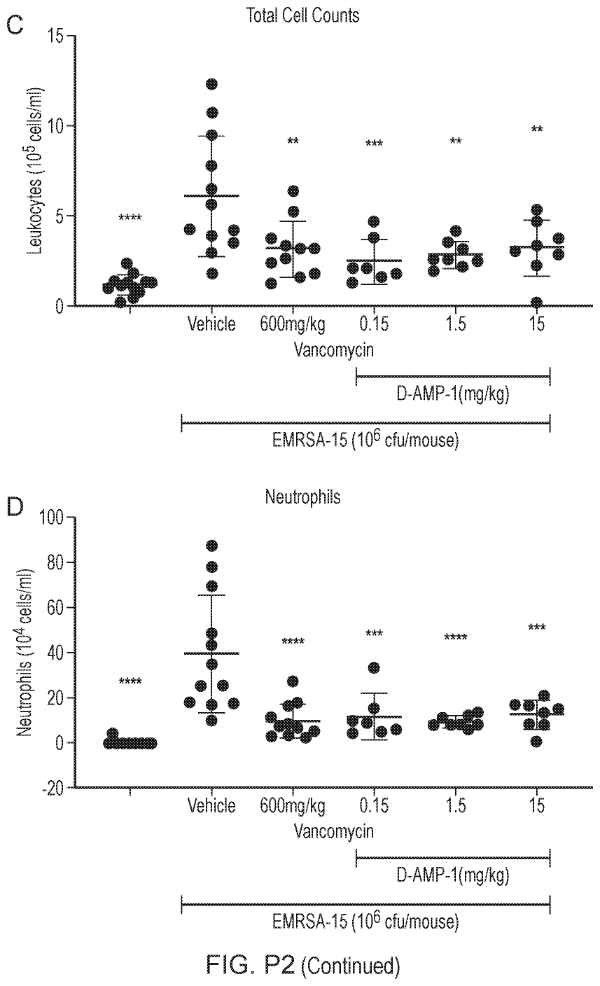

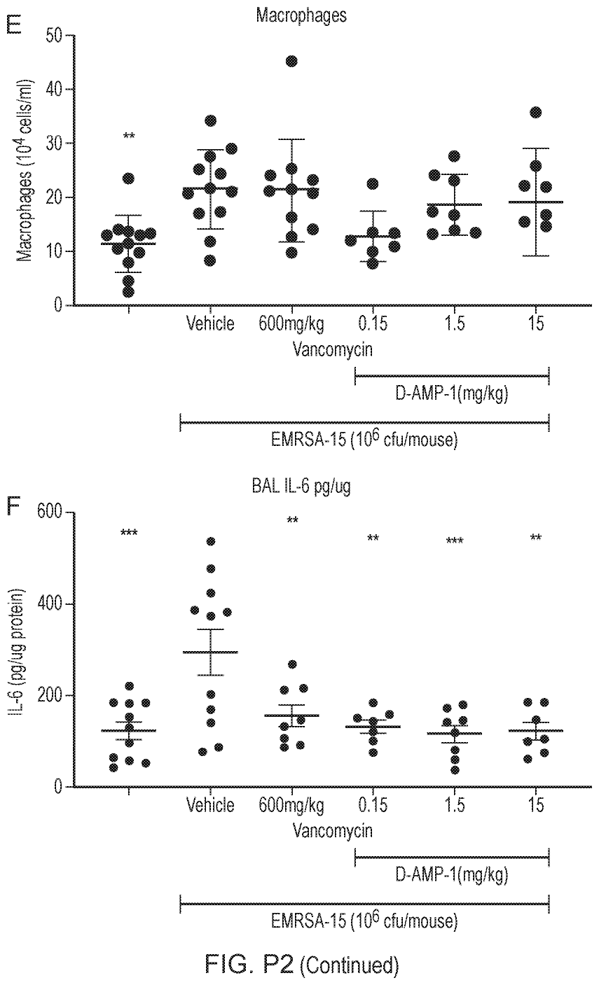

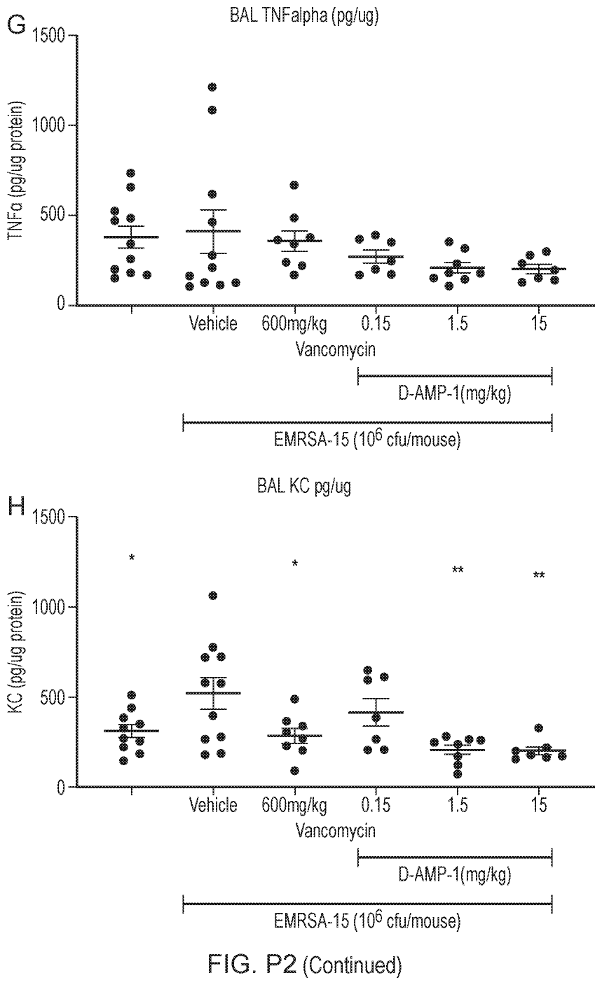

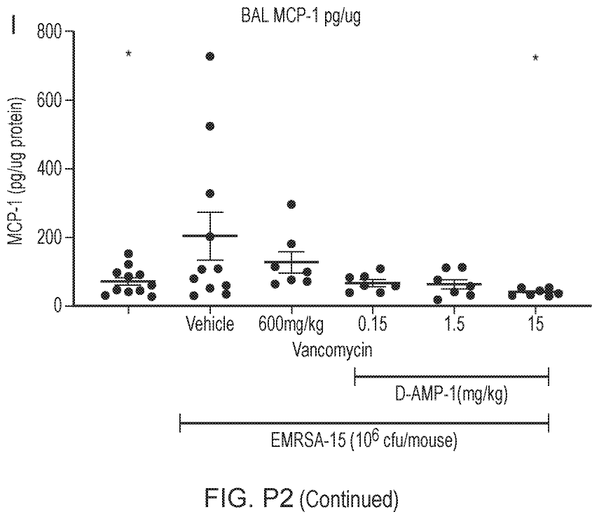

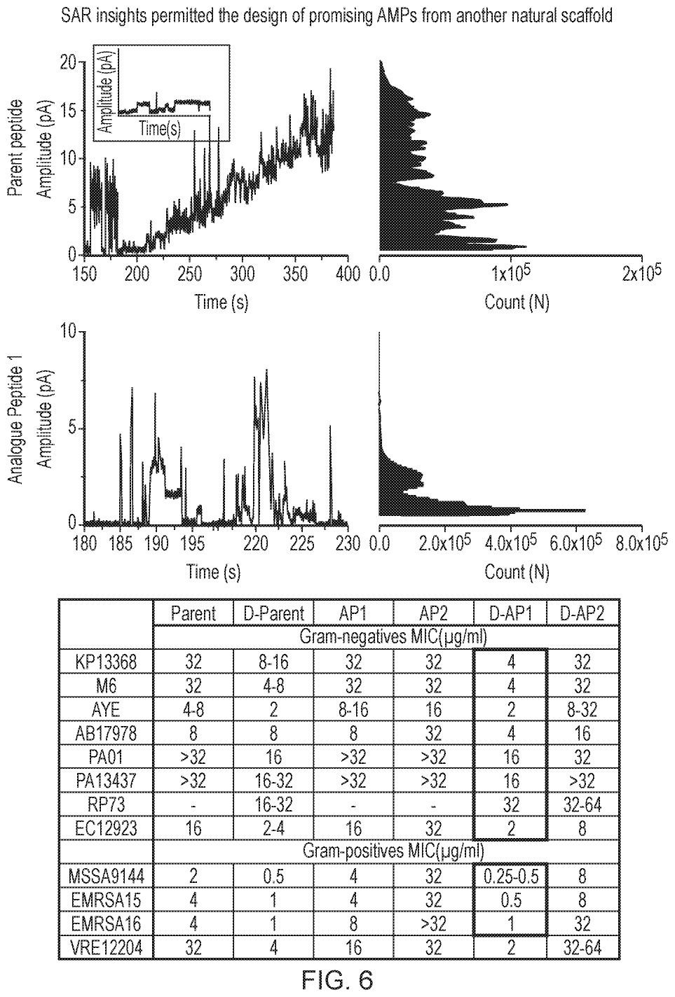

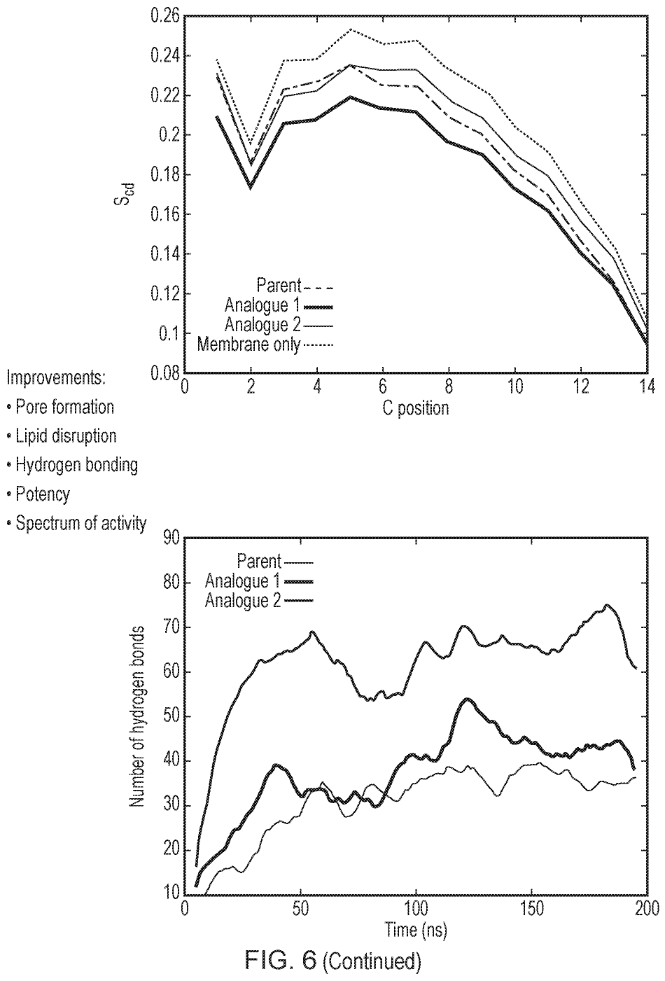

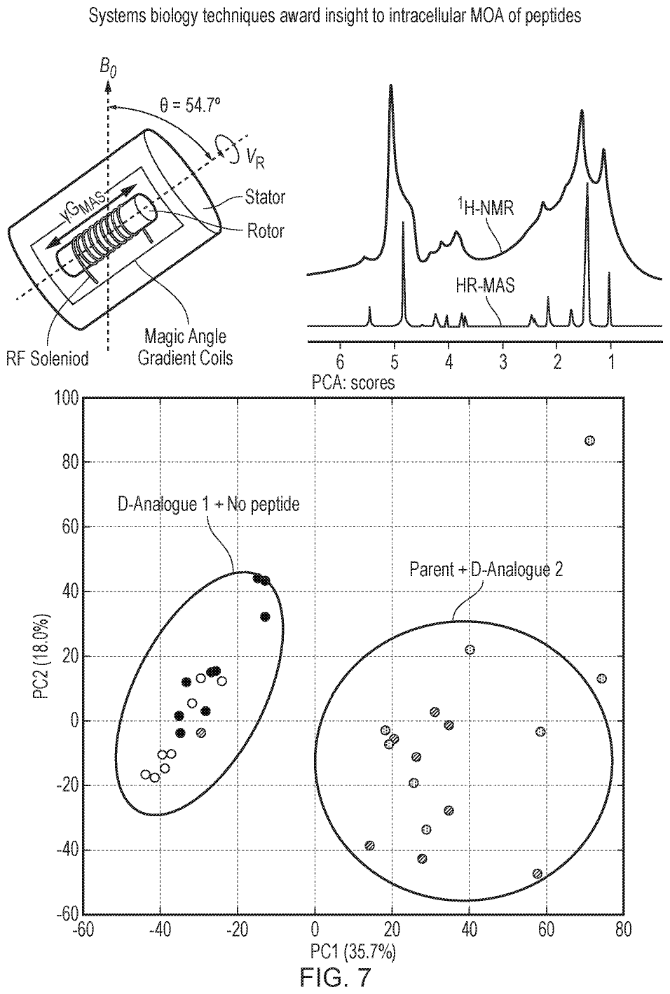

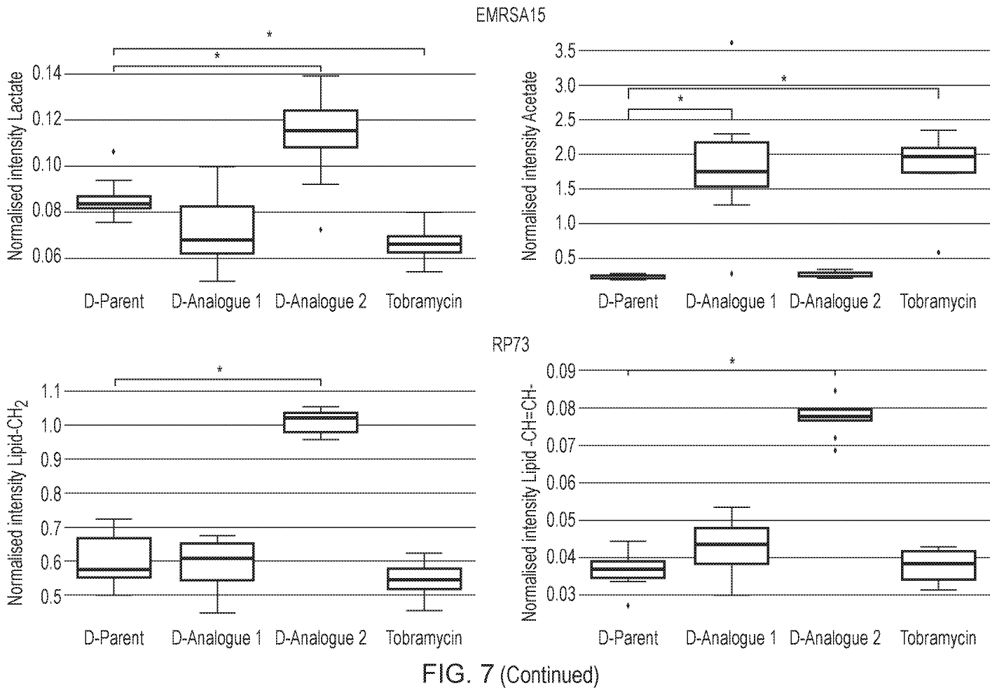

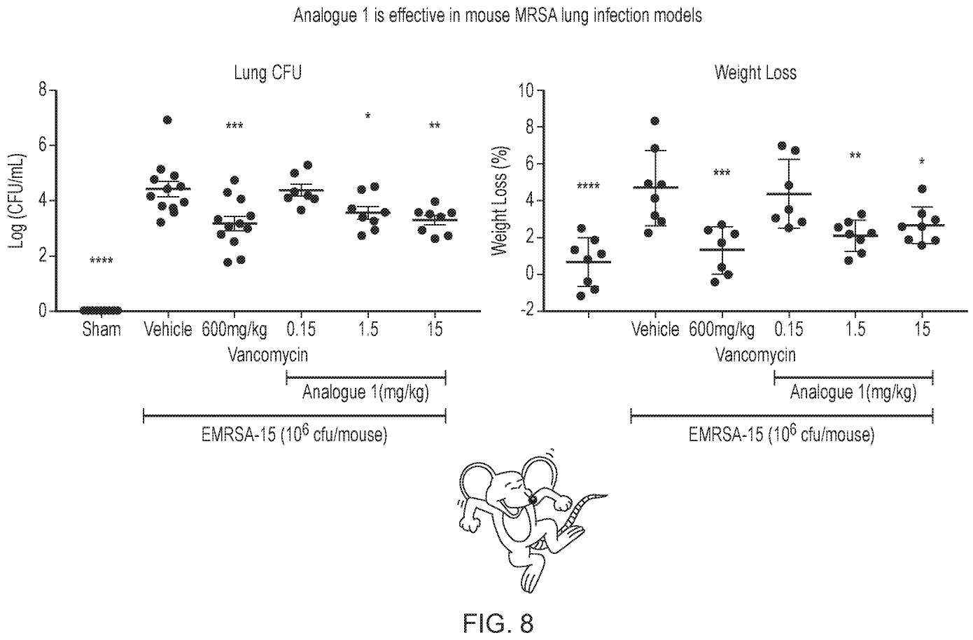

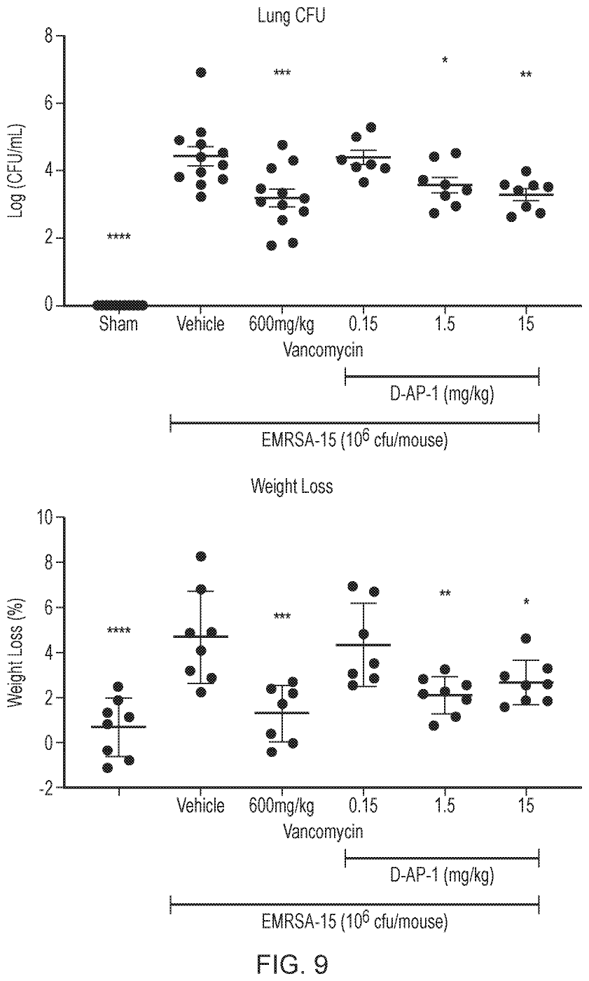

### EXAMPLES

### Example 1: Antimicrobial Peptide Analogues with Therapeutic Potential In Vitro and In Vivo

Methods: The natural AMP (termed AMP1) was modified to produce synthetic variants, AMPs 1a, 2a, 2b, 3a and 3b. Minimum inhibitory concentrations (MICs) were determined using the microbroth dilution assay with Mueller Hinton broth (MHB) or RPMI (with 5% FBS) and stability in serum was tested by conducting MICs in MHB containing 10% FBS. Synergy experiments were performed following a checkerboard protocol. Cytotoxicity was tested against HEK293 and HeLa immortalised human cell-lines using the XTT viability stain and by quantifying haemolysis of human red blood cells. Patch clamp analysis was performed using the Port-a-Patch® automated patch-clamp system on DPhPE/DPhPG (model for a Gram-negative plasma membrane) or DPhPG (Gram-positive) model membranes challenged with peptide. The murine lung infection model was performed using the agar bead model with Staphylococcus aureus strain NCTC 13616 (EMRSA-15). Mice were dosed with vancomycin or peptide in PBS 3 times over a 48-hour period; lung CFU, weight loss, immune cells in the bronchoalveolar lavage fluid and levels of the cytokines IL-6, TNFα, KC and MCP-1 were monitored.

Results: In MHB, AMP1a and AMP1b showed broad-spectrum activity against Gram-positive and Gram-negative bacteria. AMP2a and AMP2b had markedly better activity against Gram-positive and Pseudomonas species, whilst AMP3a and AMP3b maintained antibacterial activity but were much less cytotoxic against human cells. In RPMI media, the MIC results showed greater discrimination between the ‘a’ and ‘b’ enantiomers of the AMPs and the peptides were up to 16-fold more potent against S. aureus than they were in MHB. None of the AMPs showed significant loss of activity in the presence of serum or notable haemolysis at concentrations at which they were antibacterial. Patch clamp analysis revealed stark differences in channel forming ability between peptides. AMP2b was selected as the peptide with the highest therapeutic index, for testing against EMRSA-15 in a murine lung infection model. Treatment with a cumulative dose of 15 or 1.5 mg/kg of AMP2b was well tolerated and reduced lung colony forming units (CFU) by a significant level compared to vehicle, with a 1.14 log reduction for 15 mg/kg comparable to vancomycin at 600 mg/kg. No evidence of up-regulation of inflammatory markers was observed suggesting direct killing of the pathogens in the lung.

### Example 2: Antimicrobial Peptide Analogues with Therapeutic Potential In Vitro and In Vivo

### INTRODUCTION

Non-traditional approaches, outside the definition of a traditional antibiotic, are being investigated for their potential impact on tackling AMR. One such approach is the use of antimicrobial peptides (AMPs), although they are often dismissed as being poorly suited for systemic infections due to poor tolerability in animal models and high susceptibility to degradation.

The natural AMP, named ‘Parent’ here, has been found to have broad antimicrobial properties against Gram-positive and Gram-negative bacteria. This activity is thought to be due to its ability to damage the plasma membrane and also to disrupt cell metabolism, with several studies determining that it crosses the plasma membrane and attacks intracellular targets.

In this study we examined the altered antimicrobial activity, cytotoxicity and membrane binding properties of analogues of the parent AMP specifically designed to have increased conformational flexibility, and their all-D enantiomer counterparts. We also examined the in vivo efficacy of one analogue against a Gram-positive lung infection when delivered intravenously.

TABLE 1
Antimicrobial activity and cellular toxicity. MS-methicillin sensitive; EMR-
epidemic methicillin resistant; VS-vancomycin sensitive; VR-vancomycin resistant.
Values are given for peptides tested in Mueller-Hinton broth with values obtained in
RPMI given in parentheses. The Selectivity Index (SI) is the EC₅₀ divided by the MIC
in the indicated conditions. Grey or bold values indicate respectively a significant
reduction or improvement in potency in RPMI.

| | | Peptide concentration (μg/ml) | | | | | |
|---|---|---|---|---|---|---|---|
| | Isolate | Parent | D-Parent | AMR-1 | D-AMP1 | AMP-2 | D-AMP-Z |
| Gram- | K. pneumoniae | 4-8 (3) | 4 (8-16) | 2-4 (32) | 2 (4) | 4-8 (32) | 8 (32) |
| negative | NCTC 13368 | | | | | | |
| | K. pneumoniae | 4 (32) | 4 (4-8) | 2 (32) | 2 (4) | 4-8 (32) | 4 (32) |
| | M6 | | | | | | |
| | A. baumannii | 1-2 (4-8) | 1 (2) | 1-2 (8-16) | 2 (2) | 2 (16) | 1 (8-32) |
| | AYE | | | | | | |
| | A. baumannii | 1-2 (8) | 1 (8) | 1 (8) | 2 (4) | 1-2 (32) | 1 (16) |
| | ATCC 13437 | | | | | | |
| | P. aeruginosa | 64 (>32) | 2 (16) | 4 (>32) | 4 (16) | 16-32 (>32) | 4 (32) |
| | PA01 | | | | | | |
| | P. aeruginosa | 16-32 (>32) | 8 (16-32) | 8 (>32) | 4 (16) | 32 (>32) | 32 (>32) |
| | NCTC 13437 | | | | | | |
| | E. coli | 1-2 (16) | 1 (2-4) | 1 (16) | 1 (2) | 1 (32) | 1 (8) |
| | NCTC 12923 | | | | | | |
| Gram- | MS S. aureus | 4 (2) | 2 (0.5) | 2 (4) | 2 (0.25-0.5) | 8 (32) | 4 (8) |
| positive | ATCC 9144 | | | | | | |
| | EMR | 16 (4) | 16 (1) | 4 (4) | 2 (0.5) | 64 (32) | 32 (8) |
| | S. aureas-15 | | | | | | |
| | EMR | 16 (4) | 16 (1) | 4 (8) | 4 (1) | 64-128 (>32) | 64 (32) |
| | S. aureas-16 | | | | | | |
| | VS E. faecalis | 64 (NG) | 32 (NG) | 16 (NG) | 8 (NG) | 64-128 (NG) | 64 (NG) |
| | NCTC 775 | | | | | | |
| | VR E. faecium | 16-32 (32) | 16 (4) | 4 (16) | 2 (2) | 32-64 (32) | 16 (32-64) |
| | NCTC 12204 | | | | | | |
| | HEK293 | (87.5 ± 11.1) | (61.4 ± 3.9) | (40.1 ± 4.1) | (36.4 ± 4.3) | (~400) | (217.3 ± 46.5) |
| Toxicity | HeLa | (58.2 ± 3.9) | (47.1 ± 2.1) | (30.7 ± 2.6) | (25.3 ± 2.2) | (>400) | (198.2 ± 10.2) |
| | HC50 | ~400 | >400 | >400 | ~400 | >>400 | >>400 |
| | HEK293/ | 5.5 (21.9) | 3.8 (61.4) | 10.0 (10.0) | 18.2 (72.8) | 6.25 (12.5) | 6.8 (27.2) |
| | EMRSA-15 | | | | | | |

Figure P1: Activity of parent peptide and its analogues on in silico and in vitro models of bacterial plasma membranes. Patch clamp: Representative current traces illustrating membrane activity when model membranes are challenged with each peptide at the indicated concentration. The highly irregular but high amplitude conductance that diminishes over time, observed for the Parent, most notably in membranes that model the Gram-negative bacterial plasma membrane and remain intact, is consistent with an AMP crossing the bilayer without major structural disruption. Such activity is also observed in models of Gram-positive plasma membranes where, additionally, more channel like conductance is observed.

Figure P2: Systemically delivered D-AMP-1 is effective in a murine model of EMRSA-15 lung infection. C57B16J mice, challenged with 1×106 cfu/mouse EMRSA-15 in tryptic soy agar beads, were treated with vancomycin or D-AMP-1 in three intravenous doses at 4, 24 and 30 hours post infection to achieve the cumulative doses indicated. Bacterial burden in the lung (A), weight loss over the infection period (B) and BAL cells (C-E) and cytokines (F-I) reveal the effect of each intervention. Significance is indicated relative to the saline vehicle (p<0.05 *; <0.01 **; 0.001 ***; 0.0001 ****).

### Conclusions

We have demonstrated that analogues of the parent AMP, in particular D-AMP-1, are potent bactericidal AMPs which can be delivered intravenously to treat bacterial lung infections without triggering the release of pro-inflammatory cytokines or stimulating recruitment of innate immune cells in the mouse model.

The biophysical analysis, taken together with previous fluorescence studies, reveal species and even strain dependent differences in the extent of membrane permeabilization caused by the peptides at their MICs. This suggests that a primary mechanism of action will be to penetrate the bacteria but at higher concentrations a secondary, membrane disruptive, effect will be observed and that optimisation of the latter function yields more robust performance in a variety of conditions.

### Example 3

A patient is diagnosed with an infection caused by one of the infectious agents described herein, in one of the clinical indications identified.

A decision to treat with the compound(s) of the invention may be chosen on an empirical basis or treatment directed following initial identification/speciation of the infectious agent and/or its antibiotic resistance profile.

The treatment is selected from one of the AMP treatment options:
monotherapy with an AMP as described herein,
combined therapy of one or more AMP as described herein with existing antibiotics, or
combination therapy with 2 or more AMPs as described herein.

The formulation and delivery method will reflect the indication, but the default will be i.v. formulation and administration.

Treatment will follow a defined schedule.

### Example 4

EMRSA-15 response to challenge with the three D-pleurocidin analogues in MH varies according to metabolism, osmoregulation and cell wall composition.

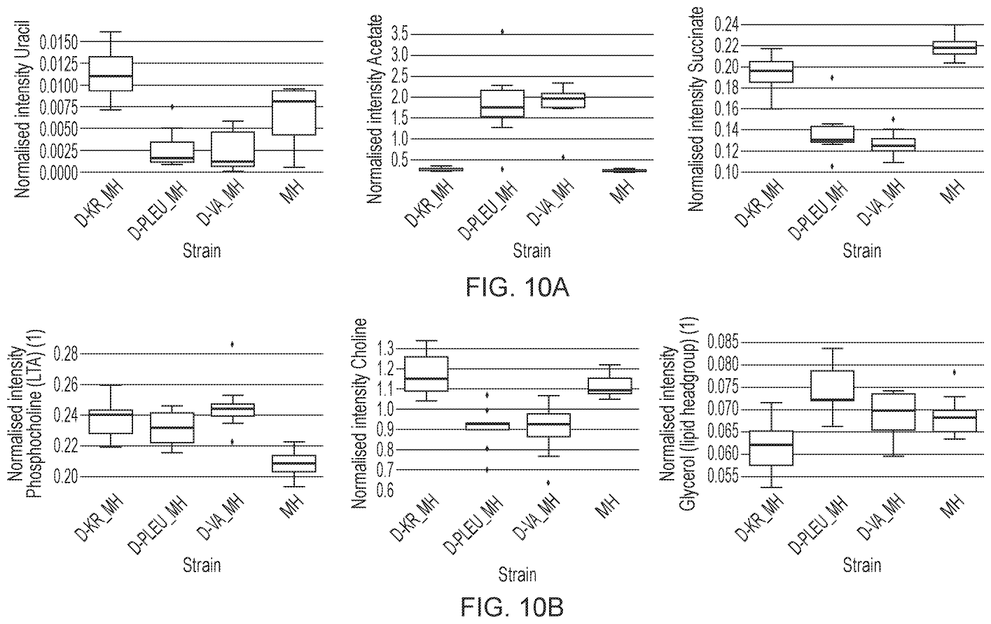

FIG. 10A shows EMRSA-15 response to is grown in the presence of sub-inhibitory concentrations of peptide in MH, D-pleurocidin (MIC—16 μg/ml) triggers somewhat different responses than D-pleurocidin-KR (MIC—2 μg/ml) or D-pleurocidin-VA (MIC—32 μg/ml). Most notably, D-pleurocidin-KR alone triggers an increase in uracil and does not trigger an increase in acetate or reduction in succinate.

FIG. 10B shows EMRSA-15 response to is grown in the presence of sub-inhibitory concentrations of peptide in MH, D-pleurocidin (MIC—16 μg/ml) triggers somewhat different responses than D-pleurocidin-KR (MIC—2 μg/ml) or D-pleurocidin-VA (MIC—32 μg/ml). All three peptides induce changes in lipoteichoic acid but other cell wall components are differentially affected. D-pleurocidin-KR induces less osmotic stress as evidence by choline.

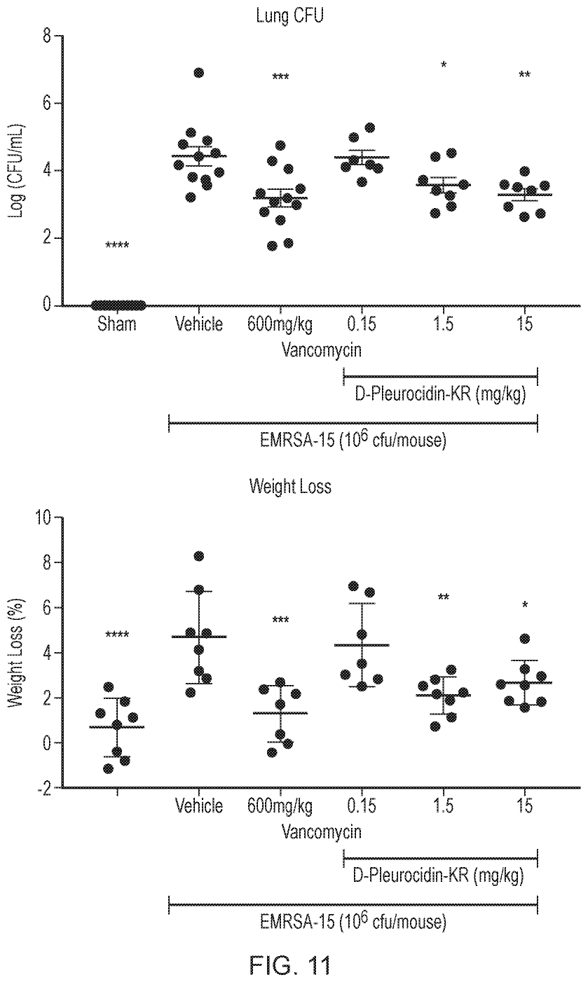

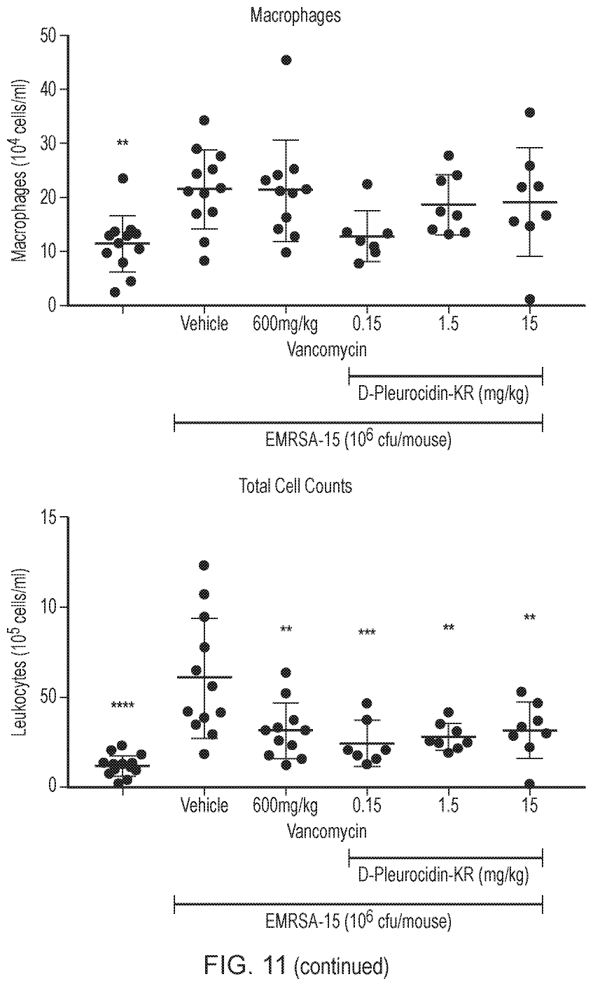

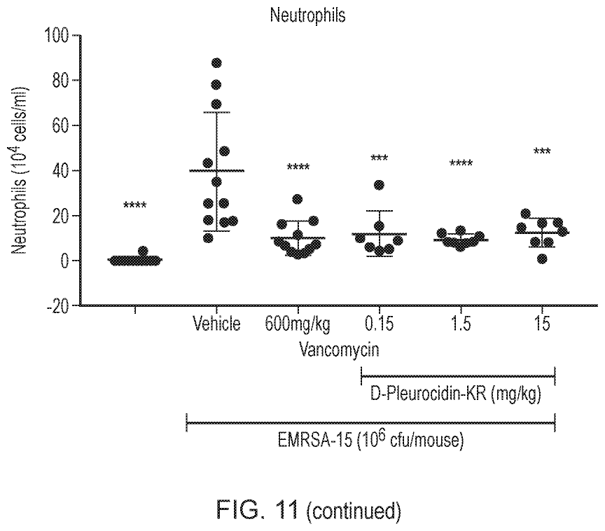

FIG. 11 shows Pleurocidin analogue is an effective therapeutic in a murine model of EMRSA-15 lung infection.

D-pleurocidin-KR delivered i.v. in three separate doses (dose shown is accumulative).

Lung CFU burden reduced by 1.1 log 10 with 15 mg/kg D-pleurocidin-KR is equivalent to that achieved with 600 mg/kg vancomycin, the current standard of care.

Weight loss and neutrophil recruitment associated with EMRSA-15 infection is mitigated in successfully treated mice.

### Example 5—Summary of Potency/Toxicity Data

The bacterial strains used are as in the above examples. A table of strains is provided below to aid understanding.

The peptides in this example are as described above.

Data is provided below.

MIC on Gram-negative and Gram-positive initial panels

| | | | Pleuro- | Pleuro- | Pleuro- | Pleuro- | D-Pleuro- | D-Pleuro- | D-pleuro- |
|---|---|---|---|---|---|---|---|---|---|
| | Pleurocidin | D-Pleuro | D1 | D2 | KR | VA | KR | VA | VAKR |
| | Gram-negatives | | | | | | | | |
| KP13368 | 4-8 | 4 | 16 | >128 | 2-4 | 4-8 | 2 | 8 | 4-8 |
| M6 | 4 | 4 | 16-32 | 128 | 2 | 4-8 | 2 | 4 | 4 |
| AYE | 1-2 | 1 | 2 | 16 | 1-2 | 2 | 2 | 1 | 2 |
| AB17978 | 1-2 | 1 | 2-4 | 16 | 1 | 1-2 | 2 | 1 | 2 |
| PA01 | 64 | 2 | 32-64 | >128 | 4 | 16-32 | 4 | 4 | 4 |
| PA13437 | 16-32 | 8 | 32 | 128 | 8 | 32 | 4 | 32 | 32 |
| RP73 | — | 8-16 | — | — | — | — | 16 | 16-64 | — |
| EC12923 | 1-2 | 1 | 2 | 8-16 | 1 | 1 | 1 | 1 | 2 |
| | Gram-positives | | | | | | | | |
| MSSA9144 | 4 | 2 | 8-16 | 16-32 | 2 | 8 | 2 | 4 | 8 |
| EMRSA15 | 16 | 16 | 64-128 | >128 | 4 | 64 | 2 | 32 | 32 |
| EMRSA16 | 16 | 16 | 128 | >128 | 4 | 64-128 | 4 | 64 | 64 |
| VSE775 | 64 | 32 | 128 | >128 | 16 | 64-128 | 8 | 64 | 32-64 |
| VRE12201 | NG | NG | >128 | >128 | 32 | >128 | 8 | 128 | 128 |
| VRE12204 | 16-32 | 16 | 32-64 | 64 | 4 | 32-64 | 2 | 16 | — |
| | Fungi | | | | | | | | |
| C. albicans | | | | | | | | 8 | 32 |
| | Toxicity | | | | | | | | |
| HEK293 | 87.5 ± 11.1 | 61.4 ± 3.9 | >400 | >400 | 40.1 ± 4.1 | ~400 | 36.4 ± 4.3 | 217.3 ± 46.5 | 201.2 ± 25.1 |
| HeLa | 58.2 ± 3.9 | 47.1 ± 2.1 | >400 | >400 | 30.7 ± 2.6 | >400 | 25.3 ± 2.2 | 198.2 ± 10.2 | 124.0 ± 30.4 |

| | | | Pleuro- | Pleuro- | Pleuro- | Pleuro- | D-Pleuro- | D-Pleuro- | D-pleuro- |
|---|---|---|---|---|---|---|---|---|---|
| | Pleurocidin | D-Pleuro | D1 | D2 | KR | VA | KR | VA | VAKR |
| | Gram-negatives | | | | | | | | |
| KP13368 | 32 | 8-16 | 64-128 | >128 | 32 | 32 | 4 | 32 | |
| M6 | 32 | 4-8 | 64-128 | >128 | 32 | 32 | 4 | 32 | |
| AYE | 4-8 | 2 | 64 | 128 | 8-16 | 16 | 2 | 8-32 | |
| AB17978 | 8 | 8 | 64 | 128 | 8 | 32 | 4 | 16 | |
| PA01 | >32 | 16 | >128 | >128 | >32 | >32 | 16 | 32 | |
| PA13437 | >32 | 16-32 | >128 | >128 | >32 | >32 | 16 | >32 | |
| RP73 | — | 16-32 | — | — | — | — | 33 | 32-64 | — |
| EC12923 | 16 | 2-4 | 32 | 128 | 16 | 32 | 2 | 8 | |
| | Gram-positives | | | | | | | | |
| MSSA9144 | 2 | 0.5 | 128 | 128 | 4 | 32 | 0.25-0.5 | 8 | |
| EMRSA15 | 4 | 1 | 128 | 128 | 4 | 32 | 0.5 | 8 | |
| EMRSA16 | 4 | 1 | 128 | >128 | 8 | >32 | 1 | 32 | |
| VSE775 | NG | NG | NG | NG | NG | NG | NG | NG | |
| VRE12201 | NG | NG | NG | NG | NG | NG | NG | NG | |
| VRE12204 | 32 | 4 | 128 | 128 | 16 | 32 | 2 | 32-64 | |
| | | | | | D- | | D- | | |
| | Pleurocidin | | Pleurocidin | | Pleurocidin | | pleurocidin | | |
| | (MH) | | (RPMI) | | (MH) | | (RPMI) | | |
| Klebsiella | 4-8 | | 32 | | 4 | | 8-16 | | |
| pneumoniae | | | | | | | | | |
| KP13368 | | | | | | | | | |

| | | | | |
|---|---|---|---|---|
| M6 | 4 | 32 | 4 | 4-8 |
| AYE | 1-2 | 4-8 | 1 | 2 |
| AB17978 | 1-2 | 8 | 1 | 8 |
| PA01 | 64 | >32 | 2 | 16 |
| PA13437 | 16-32 | >32 | 8 | 16-32 |
| EC12923 | 1-2 | 16 | 1 | 2-4 |

Stability in serum (MH)

| | Pleurocidin | | D-pleurocidin | | D-pleurocidin-KR | | Pleurocidin-KR | | Pleurocidin-VA | | D-Pleurocidin-VA | |
|---|---|---|---|---|---|---|---|---|---|---|---|---|
| | No | | No | | No | | No | | No | | No | |
| | serum | Serum | serum | Serum | serum | Serum | serum | Serum | serum | Serum | serum | Serum |
| KP13368 | 4 | 8 | 4 | 4 | 2 | 1 | 2 | 4 | 8 | 16 | 8 | 8-32 |
| M6 | 4 | 8 | 4 | 4 | 2 | 0.5 | 4 | 2 | 16 | 8 | 4 | 8 |
| AYE | 2 | 2 | 2 | 2 | 2 | 0.5 | 2 | 2 | 2 | 4 | 1 | 2 |
| Ab17978 | 2 | 2 | 1 | 0.5 | 2 | 0.5 | 2 | 2 | 2 | 4 | 1 | 2-16 |
| PA01 | 16 | 32 | 2 | 4 | 4 | 4 | 4 | >128 | 16* | >128 | 4 | 16 |
| PA13437 | 16 | 32 | 8 | 16 | 4 | 8 | 4 | 32* | 16* | >128 | 32 | 32 |
| EC12923 | 2 | 1 | 1 | 0.5 | 1 | 0.25 | 2 | 1 | 1 | 1 | 1 | 1 |

For the most part, the D-AMPs appear to retain activity in the presence of 10% fetal bovine serum. Activity is lost for L-AMPs for Pseudomonas but not other species.

The underlined values indicates these peptides perform at a lower level against the same strains in RPMI compared to in MHB. It does not indicate that they lack function (although in some cases activity is poor).

As noted in the description, the peptides Pleuro-D1 and Pleuro-D2 (‘mixed peptides’ containing both D- and L-amino acids) are suitably not part of the invention.

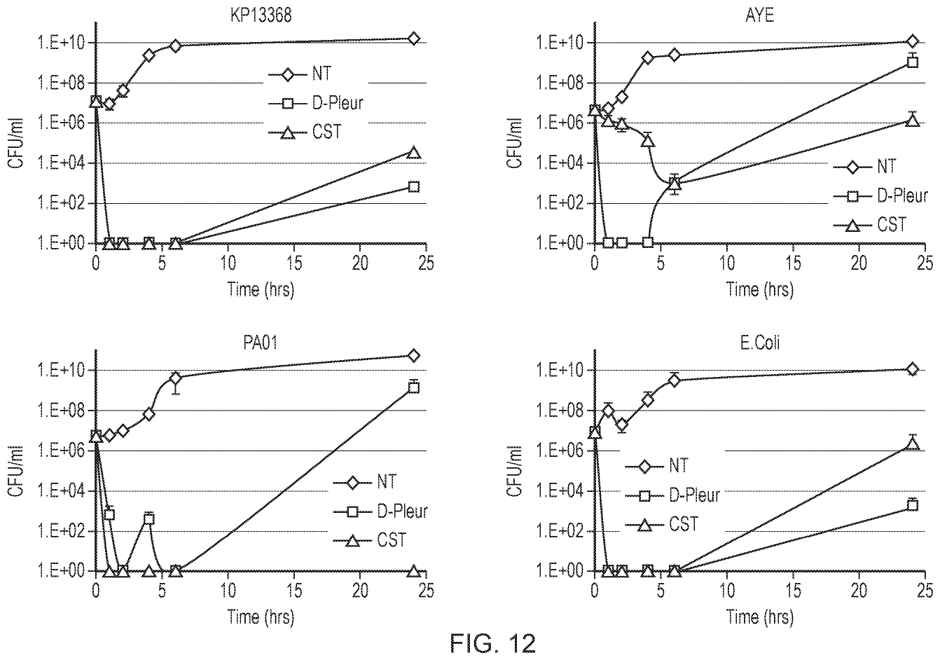

We refer to FIG. 12 which shows graphs of time kills.

TABLE Of Bacterial Strains Used

| Species | Strain | Antibiotic resistance profile |
|---|---|---|
| Klebsiella | NCTC 13368 | AMP, PIP, GEN, TOB, |
| pneumoniae | M6 | CTX, CAZ, ATM, CHL |
| | | AMP, PIP, GEN, TOB |
| Acinetobacter | NCTC 17978 | Sensitive |
| baumannii | AYE (ATCC | AMP, GEN, CIP, TZP, |
| | BAA-1710) | LVX, AMK |
| Pseudomonas | PAOI (ATCC | AMP, PIP, ATM, CHL |
| aeruginosa | BAA-47) | |
| | NCTC 13437 | AMK, GEN, TOB, AMP, PIP, |
| | | TZP, CAZ, ATM, IMP, |
| | | MEM, CIP, LVX, CHL |
| Escherichia coli | NCTC 12923 | AMK, GEN, TOB |
| Enterococcus | NCTC 12204 | VAN, CIP |
| faecalis | NCTC 12201 | VAN, CIP |
| Enterococcus | NCTC 775 | Susceptible |
| faecium | | |
| Staphylococcus | EMRSA-15 | MET, CIP |
| aureus | (NCTC 13616) | |
| | EMRSA-16 | MET, CIP |
| | (NCTC 13277) | |
| | ATCC 9144 | Susceptible |
| | (NCTC 6571) | |

Abbreviations as standard in the art (e.g. according to BSAC Bacteraemia Resistance Surveillance Programme'/‘BSAC Respiratory Resistance Surveillance Programme’ published by the British Society for Antimicrobial Chemotherapy, Griffin House, 53 Regent Place, Birmingham, B1 3NJ, UK)—http://www.bsacsurv.org/science/antimicrobials/

### Example 6: D-PLEUROCIDIN-KR

Here we demonstrate the effectiveness of peptides according to the present invention, using D-PLEUROCIDIN-KR as an illustrative example.

We evaluate the in vitro potency of D-PLEUROCIDIN-KR. D-PLEUROCIDIN-KR is a 25 residue amphipathic peptide comprising all D-amino acids and aminated at the C-terminus. D-PLEUROCIDIN-KR has a broad spectrum activity and can act in synergy with the aminoglycoside tobramycin and with rifampin, with activity against Klebsiella pneumoniae, Acinetobacter baumannii and Escherichia coli, including antibiotic resistant strains. Potency towards Pseudomonas aeruginosa is a little lower but enhanced in combination with either tobramycin or rifampin.

In this example, D-PLEUROCIDIN-KR was tested with five S. aureus strains. Three studies were conducted: (1) The MIC values were determined with a collection of five S. aureus strains using two types of medium, Mueller Hinton Broth and RPMI 1640 containing 5% fetal bovine serum. The MIC study included linezolid and vancomycin as quality control reference agents. (2) A Maximum Tolerated Dose (MTD) analysis of D-PLEUROCIDIN-KR was conducted with intravenous (IV) dose administration to determine the upper limit of dosing in immunocompetent mice.

### Materials

Chemicals and Medium: Bacto agar (214040, BD, USA), Bacto agar (214040, BD, USA), Brain-heart infusion broth (BHI) (237500, BD, USA), Cyclophosphamide monohydrate (C-0768, Sigma, USA), Etomidate-®lipuro (20 mg/10 mL; B. Braun Melsungen AG, Germany), Fetal Bovine Serum (SH30084.03 HyClone, USA), Difco™ Mueller Hinton Broth II (275730, BD, USA), Linezolid (GA2609, Glentham Life Science, UK), Nutrient agar (NA) plates (CMP0101312, CMP, Taiwan), Nutrient broth (DIFCO, USA), Phosphate-buffered saline (PBS) tablet (P4417, Sigma, USA), Saline (sterilized 0.9% NaCl, 53539, SinTong, Taiwan), RPMI 1640 (SH30027.02, HyClone, USA), Vancomycin hydrochloride (V2002, Sigma, USA), and Water for injection (WFI, sterilized water, Tai-Yu, Taiwan).

Plasticware: 96-well V-bottom polypropylene plate (Nunc™ 249949, Nunc, USA), Biomek tip (717252, Beckman Coulter, USA), Counting vial (Hsin-Pei Co, Ltd., Taiwan), Disposal syringe (1 mL, Terumo, Japan), Microcentrifuge Tubes 1.5 mL Click-Cap (Treff AG, Switzerland), Pipets (Costar, USA), Sterile inoculation loop (731161, Bio-Check Laboratories, Taiwan) and Tip (Labcon, USA).

### Equipment

Absorbance microplate readers (Tecan, Infinite F50, USA), Biomek 96 well liquid handling instruments (Beckman Coulter, FX and FXP, USA), Biological safety cabinet (NuAire, USA), Centrifuge (Model 5810R, Eppendorf, Germany), Centrifuge (Model 5922, Kubota, Japan), Electronic scale (Tanita, Model 1140, Japan), Incubators (Firstek, Taiwan), Individually Ventilated Cages (GM500 IVC seal safe plus cage system) (Tecniplast, Italy), Micropipettes (Gilson, France), Open-Topped cage (Allentown, USA), Orbital shaking incubator (Firstek Scientific, Taiwan), Pipetman (Rainin, USA), Pipetman (P100 Gilson, France), Polytron homogenizer (Kinematica, Switzerland), Portable weighing scale (JKH-1000, Jadever, Taiwan), Refrigerated incubator (Hotpack, USA), Shaking incubator (Firstek, Taiwan), Refrigerator-freezer (Sanyo, Japan), and Ultra-Low temperature freezer (Panasonic, Japan).

### Microbial Strains

The S. aureus strains ATCC 29213, ATCC 19636, ATCC 33591 (MRSA), BAA-1556 (MRSA USA 300) were obtained from the American Type Culture Collection (ATCC). Vancomycin-resistant S. aureus VRS-2 (VanA VRSA) was obtained from the Network for Antibiotic Resistance in S. aureus (NARSA). The mouse lung infection models have been validated with these strains. The antibiotic susceptibility of these strains is summarized in the Table below and was determined following the recommend assay conditions of the Clinical Laboratory Standards Institute (CLSI) M7-A10 microdilution procedure and the CLSI M100 interpretive criteria. The species identity was confirmed with 16S rRNA analysis.

TABLE
S. aureus strains for MIC studies, antibiotic susceptibility

| | Source strain ID | | | | | | | | | |
|---|---|---|---|---|---|---|---|---|---|---|
| | | | ATCC | | | | | | | |
| | ATCC | | 19636 | | ATCC | | | | | |
| | 29213 | | (Smith) | | 33591 | | BAA-1556* | | VRS-2 | |
| | Resistance | | | | | | | | | |
| | | | | | | | MRSA | | VanA | |
| | Susceptible | | Susceptible | | MRSA | | USA 300 | | VRSA | |
| Oxacillin | 0.25-0.5 | S | 0.25 | S | >64 | R | 64 | R | >64 | R |
| Cefepime | 2-4 | — | 2 | — | >64 | — | 16-64 | — | >64 | — |
| Ceftriaxone | 4 | — | 4 | — | >64 | — | ≥64 | — | >64 | — |
| Imipenem | 0.016-0.031 | — | ≤0.016 | — | ≥16 | — | 0.125-0.25 | — | >16 | — |
| Meropenem | 0.0625 | — | — | — | — | — | — | — | — | — |
| Vancomycin | 0.5-1 | S | 1 | S | 1 | S | 0.5-1 | S | >64 | R |
| Teicoplanin | 0.25 | S | — | — | — | — | — | — | — | — |
| Linezolid | 2-4 | S | 2-4 | S | 0.5-1 | S | 2 | S | 2 | S |
| Daptomycin | 0.125-0.5 | S | 0.25 | S | 0.5 | S | 0.25 | S | 0.25 | S |
| Tigecycline | 0.125-0.25 | — | 0.125-0.25 | — | 0.25-0.5 | — | 0.25 | — | 0.25 | — |
| Trimethoprim/ | ≤0.5/9.5 | S | ≤0.5/9.5 | S | 0.5/9.5 | S | 0.25/4.75 | S | >8/152 | R |
| sulfamethoxazole | | | | | | | | | | |
| Clindamycin | 0.125 | S | 0.125 | S | >64 | R | >64 | R | >64 | R |
| Erythromycin | 0.25-1 | S-I | 0.25-1 | S-I | >64 | R | >64 | R | >64 | R |
| Gentamicin | 0.25 | S | 0.25 | S | 0.25-1 | S | 0.25-0.5 | S | 32-64 | R |
| Mupirocin | 0.25-2 | — | 1 | — | 1-2 | — | >64 | — | 1-2 | — |
| Levofloxacin | 0.125-0.25 | S | 0.125 | S | 0.25 | S | 4-8 | R | 32 | R |

S denotes susceptible, R denotes resistant or not susceptible, and I denotes intermediate susceptibility based on the CLSI interpretive criteria published in CLSI M100.
*BAA-1556 is a mupirocin resistant strain.

D-PLEUROCIDIN-KR solutions for MIC assessment were prepared in sterilized water for injection (WFI). Solutions for MTD and efficacy assessments were prepared in normal saline (0.9% NaCl). The dose formulation for in vivo studies was freshly prepared on the day of the study, aliquoted and then stored at 4° C. for each dose administration. All solutions were prepared as described below. The dosing volume was 5 mL/kg for intravenous (IV) dosing, and 10 mL/kg for subcutaneous (SC) dosing.

For MIC assessment, the 1.6 mg/mL D-PLEUROCIDIN-KR stock solution was freshly prepared by dissolving 1 mg of D-PLEUROCIDIN-KR in 0.625 mL of WFI directly in the vial.

For MTD assessment, the 6 mg/mL D-PLEUROCIDIN-KR solution was freshly prepared by dissolving 16.2 mg of D-PLEUROCIDIN-KR in 2.7 mL of saline. A 0.9 mL of the 6 mg/mL solution was further diluted by adding 1.8 mL of saline, generating a lower 2 mg/mL dose solution. A 0.9 mL of the 2 mg/mL solution was further diluted by adding 0.9 mL of saline, generating a lower 1 mg/mL dose solution.

### Experiment Design

a. Minimum Inhibitory Concentration (MIC) Assay

The MIC assay was used to assess the in vitro antimicrobial potency of D-PLEUROCIDIN-KR following the protocol M07-A10 of the Clinical and Laboratory Standards Institute (CLSI) for medium, inoculum preparation, and MIC endpoint reading. In this assay, the MIC value was the lowest concentration of the test agent that completely inhibits visible growth of the pathogen in broth culture. Two broth mediums were used, Mueller Hinton Broth and RPMI 1640+5% fetal bovine serum. Test substances were evaluated in 11-point titrations and performed in duplicate. D-PLEUROCIDIN-KR was evaluated at 32 to 0.0313 μg/mL, since D-PLEUROCIDIN-KR has a MIC of 4 μg/mL for epidemic methicillin-resistant S. aureus (EMRSA-15) in MHB (see examples above). Linezolid and vancomycin were evaluated as the historical reference compound.

b. Maximum Tolerated Dose (MTD) Assessment

Tolerability analysis was conducted to determine the upper limit of D-PLEUROCIDIN-KR dosage. Test article solutions were intravenously (IV) administered to immunocompetent ICR mice with the dose schedule and concentrations indicated in the study design in the table below.

TABLE
Maximum tolerability assessment (MTD), Study design

| | | | | | Dosage | | | |
|---|---|---|---|---|---|---|---|---|
| | Test | Dose | Dose | Conc. | | mg/kg/ | mg/kg/ | Mice |
| Group | Substance | Schedule | Route | mg/mL | mL/kg | dose | 24 h | female |
| 1 | Vehicle | TID | IV | NA | 5 | — | — | 4 |
| | (0.9% NaCl) | | | | | | | |
| 2 | D- | TID | IV | 6 | 5 | 30 | 90 | 4 |
| | PLEUROCI | | | | | | | |
| | DIN-KR | | | | | | | |
| 3 | D- | TID | IV | 2 | 5 | 10 | 30 | 4 |
| | PLEUROCI | | | | | | | |
| | DIN-KR | | | | | | | |
| 4 | D- | TID | IV | 1 | 5 | 5 | 15 | 4 |
| | PLEUROCI | | | | | | | |
| | DIN-KR | | | | | | | |

| | |
|---|---|
| Immunocompetent female ICR mice: | 20 |

The immunocompetent animals were not infected.
The vehicle was 0.9% NaCl.
The animals were administered test article thrice per day (TID) at 8 h intervals and
clinical symptoms associated with overt toxicity were monitored 5 minutes after each
dose (full observation after the 1^{st} dose and cage-side observation after the 2^{nd} and 3^{rd} doses).
All four groups were conducted in parallel and if overt toxicity was observed after the 1^{st} or 2^{nd} dose of a group, the 2^{nd} or 3^{rd} dose of that group was not administered.

The animals were not infected. Doses were IV administered thrice (TID) with 8 h intervals (q8h) between doses. Animals were humanely euthanized after 3 days of the last treatment or at humane endpoints.

### Methods

a) Minimum Inhibitory Concentration (MIC) Study

The direct colony suspension method was used to prepare inoculated broth. Isolated colonies were taken from an 18-24 h culture plate. Optical density measurements (OD₆₂₀ₙₘ) were used to estimate the bacterial density. The D-PLEUROCIDIN-KR, vancomycin and linezolid stock solutions were prepared as above. The stock solutions were diluted by 2-fold serial titrations with their respective vehicles for a total of 11 concentrations.

A 4 μL aliquot of each dilution was added to 196 μL of broth medium seeded with the organism in wells of a sterilized 96 well polypropylene plate (Nunc™ 249949) using aluminum foil as cover. The final bacterial count was 2 to 8×10⁵ CFU/mL. The MIC assay was conducted twice with different mediums: Mueller Hinton Broth and RPMI 1640 with 5% FBS. The final test article concentration range was 32 to 0.0313 μg/mL.

Each test substance dilution was evaluated in duplicate on one test occasion. Vehicle-control and reference control agents, linezolid and vancomycin, were used as blank and positive controls, respectively. Plates were incubated at 35-37° C. for 18 h. The test plates were visually examined and each well was visually scored for growth or complete inhibition of growth. The MIC value was recorded. The CLSI guidelines of 100% visual growth inhibition were used to call an MIC endpoint.

b) MTD Assessment of D-PLEUROCIDIN-KR

i. Animal Preparation

The MTD assessment was performed with immunocompetent female ICR mice, weighing 22 f 2 g. All animals were specific pathogen free.

ii. Treatment

Test articles were administered to animals by IV injections following the dose schedule, volumes, and concentrations indicated the Table above for MTD study. Animals were observed for 5 min after each administration to detect acute toxicity which was recorded and reported, if observed. Animals were humanely euthanized if severe acute toxicity was observed during the experimental period.

iii. Dosing and Animal Observations

Clinical examinations: animals were observed for the presence of acute toxic symptoms and autonomic effects during the first 5 minutes after the first IV administration—see table below:

Table: Clinical observations

| | Decrease in | Decrease in | | |
|---|---|---|---|---|
| Body Weight | Touch | Spontaneous | Low | Body |
| (B.W.) (g) | Response | Activity | Limb Post | Temperature |
| Irritability | Increase in | Straub Tail | Skin Color | Piloerection |
| | Exploration | | | |
| Hyperactivity | Decrease in | Reactivity | Respiration | Increase in |
| | Exploration | | | Palpebral Size |
| Increase in | Pinna | Righting | Salivation | Decrease in |
| Startle | | | (Fluid and | Palpebral Size |
| Response | | | Viscosity) | |
| Increase | Placing | Ataxia | Lacrimation | Death |
| Touch | | | | |
| Response | | | | |
| Decrease | Tremor | Convulsion | Diarrhea | |
| Startle | | (Chronic/ | | |
| Response | | Tonic) | | |

Clinical observations were made at 5 minutes after the first dosage and recorded.

The adverse effects were monitored at 5 minutes via cage-side observation after each subsequent IV administration on Day 1. Animals were observed again for mortality at 72 h after the last treatment. Body weights were recorded before each treatment and at 72 h after the last treatment. If overt toxicity was observed after the 1^{st} or 2^{nd} dose of a group, the 2^{nd} or 3^{rd} dose of that group was not administered. Animals were humanely euthanized at earlier time points if they were found to be in distress or at a moribund state during observation.

### Data and Analysis

Any adverse events observed were summarized. List of scores from observation assessments were also recorded. The maximum dose that did not result in death or a humane euthanasia endpoint was recorded and reported.

Relevant ethical approvals were sought and obtained. Animal husbandry, welfare and care was carried out according to standard methods and complied with the relevant approvals and guidelines in place.

### Study Results and Discussion

a. MIC assessment with D-PLEUROCIDIN-KR

The MIC values of D-PLEUROCIDIN-KR and reference agents, linezolid and vancomycin, are summarized in Table MIC. The MIC values of D-PLEUROCIDIN-KR against the five tested S. aureus strains were all 2 μg/mL in Mueller Hinton Broth. The MIC value of D-PLEUROCIDIN-KR against the five tested S. aureus strains ranged from 0.125 to 0.5 μg/mL in RPMI 1640 with 5% fetal bovine serum. The S. aureus strains ATCC 29213 (susceptible strain) and ATCC BAA-1556 (USA300, MRSA resistant) both had a MIC value of 0.5 μg/mL, and strain ATCC 33591 (MRSA) had a MIC value of 0.25 μg/mL. The other two S. aureus strains had a MIC value of 0.125 μg/mL.

The MIC data of D-PLEUROCIDIN-KR using Mueller Hinton Broth as well as RPMI 1640 with 5% fetal bovine serum is consistent with the results in the earlier examples, with the tested S. aureus strains. The increased potency of D-PLEUROCIDIN-KR of culturing in RPMI with 5% FBS compared to Mueller Hinton Broth is also consistent with the earlier examples. The MIC of linezolid and vancomycin was determined for a quality control and the MIC value in Mueller Hinton Broth met the acceptance criteria based on PDS historical reference data.

b. MTD assessment of D-PLEUROCIDIN-KR

The MTD of D-PLEUROCIDIN-KR was assessed in immunocompetent ICR mice. The animals were not infected. D-PLEUROCIDIN-KR at 5, 10 and 30 mg/kg was IV administered thrice (TID) with 8 h intervals (q8h) for 1 day. Animals were monitored for acute symptoms (clinical observations table above) after the first dose administration. Clinical symptoms were scored after the first dose administration in Table MTD. The toxicity results of D-PLEUROCIDIN-KR in immunocompetent ICR mice are summarized in Table MTDR1. The body weight of all animal groups was recorded and summarized in Table MTDR2.

D-PLEUROCIDIN-KR was tolerated in immunocompetent ICR mice at 5 mg/kg (IP, TID, q8h), however the animals had severe cyanosis, labored respiration and edema after the first dose administration (Table MTD). We therefore infer that the 5 mg/kg dose may be close to MTD. D-PLEUROCIDIN-KR was not tolerated at 10 and 30 mg/kg dosages (Table MTDR1). All animals were found dead within 5 minutes after the ₁st dosing of 30 mg/kg D-PLEUROCIDIN-KR. In the 10 mg/kg D-PLEUROCIDIN-KR dosing group, one of the four animals was found dead after the 1^{st} dosing, and the surviving three animals exhibited adverse side effects; hence the 2^{nd} and 3^{rd} 10 mg/kg doses were not administered.

TABLE MIC
MIC values of D-PLEUROCIDIN-KR, linezolid and vancomycin against five S. aureus strains

| | | | | | MIC, μg/mL | | | | | |
|---|---|---|---|---|---|---|---|---|---|---|
| | | | | | PT #1235860 | | Reference | | | |
| | | | | | D-PLEUROCIDIN-KR | | Vancomycin | | Linezolid | |
| | | | | | | RPMI + | | RPMI + | | RPMI + |
| No. | Assay # | Species | Strain ID | Resistance | MHB | 5% FBS | MHB | 5% FBS | MHB | 5% FBS |
| 1 | 604110 | Staphylococcus aureus | ATCC 29213 | Susceptible | 2 | 0.5 | 0.5 | 2 | 4 | 2 |
| 2 | 606000 | Staphylococcus aureus | ATCC 19636 (Smith) | Susceptible | 2 | 0.125 | 0.5 | 2 | 2 | 2 |
| 3 | 605000 | Staphylococcus aureus | ATCC 33591 | MRSA | 2 | 0.25 | 1 | 4 | 2 | 1 |
| 4 | 604055 | Staphylococcus aureus | ATCC BAA-1556 | USA300, MRSA | 2 | 0.5 | 0.5 | 2 | 2 | 2 |
| 5 | 604147 | Staphylococcus aureus | VRS-2 | VanA, VRSA | 2 | 0.125 | 4 | >32 | 2 | 1 |

*BAA-1556 is a mupirocin resistant strain (MRSA).

TABLE MTD
MTD results, adverse effects at 5 minutes after dose administration (full observation after the 1^{st} dosing)

| | Treatment | | | | | | | | | | | | | | | |
|---|---|---|---|---|---|---|---|---|---|---|---|---|---|---|---|---|
| | | | | | PT# 1235860 | | | | | | | | | | | |
| | Vehicle | | | | (NID-162) | | | | | | | | | | | |
| | (0.9% NaCl) | | | | (D-PLEUROCIDIN-KR) | | | | | | | | | | | |
| | Route | | | | | | | | | | | | | | | |
| | IV | | | | IV | | | | | | | | | | | |
| | Dosage | | | | | | | | | | | | | | | |
| | 5 mL/kg, TID | | | | 5 mg/kg, TID g8h | | | | 10 mg/kg, TID q8h | | | | 30 mg/kg, TID q8h | | | |
| | BEHAVIORAL | | | | | | | | | | | | | | | |
| | No. 1 | No. 2 | No. 3 | No. 4 | No. 1 | No. 2 | No. 3 | No. 4 | No. 1 | No. 2 | No. 3 | No. 4 | No. 1 | No. 2 | No. 3 | No. 4 |
| B.W. (g) | 25 | 24 | 24 | 24 | 25 | 25 | 23 | 25 | 22 | 23 | 23 | 22 | 21 | 22 | 23 | 24 |
| Irritability | − | − | − | − | − | − | − | − | − | − | − | − | | | | |
| Hyperactivity | − | − | − | − | − | − | − | − | − | − | − | − | | | | |
| Inc. Startle | − | − | − | − | − | − | − | − | − | − | − | − | | | | |
| Inc. Touch | − | − | − | − | − | − | − | − | − | − | − | − | | | | |
| Dec. Startle Response | − | − | − | − | − | − | − | − | ± | − | ± | − | | | | |
| Dec. Touch Response | − | − | − | − | − | − | − | − | ± | ± | + | ± | | | | |
| Inc. Exploration | − | − | − | − | − | − | − | − | − | − | − | − | | | | |
| Dec. Exploration | − | − | − | − | − | − | − | − | + | ± | + | + | | | | |
| Pinna | − | − | − | − | − | − | − | − | + | ± | + | + | | | | |
| Placing | − | − | − | − | − | − | − | − | ± | ± | − | − | | | | |

NEUROLOGIC

| | | | | | | | | | | | | |
|---|---|---|---|---|---|---|---|---|---|---|---|---|
| Tremor | − | − | − | − | − | − | − | − | − | − | − | − |
| Dec. Spont. Activity | − | − | − | − | − | − | − | − | ± | ± | ± | ± |
| Straub Tail | − | − | − | − | − | − | − | − | − | − | − | − |
| Reactivity | − | − | − | − | − | − | − | − | − | − | − | − |
| Righting | − | − | − | − | − | − | − | − | − | − | − | − |
| Ataxia | − | − | − | − | − | − | − | − | ± | ± | ± | ± |
| Convulsion C.T.C-T | − | − | − | − | − | − | − | − | − | − | − | − |
| Low Limb Post | − | − | − | − | − | − | − | − | − | ± | ± | ± |
| Abdominal Tone | − | − | − | − | − | − | ± | − | ± | − | + | ± |
| Limb Tone | − | − | − | − | − | − | − | − | + | + | + | + |
| Grip Strength | − | − | − | − | − | − | − | − | ± | ± | − | − |

AUTONOMIC

| | | | | | | | | | | | | | | |
|---|---|---|---|---|---|---|---|---|---|---|---|---|---|---|
| Skin Color | − | − | − | − | C± | C± | C± | C± | C± | C± | C± | C± | | |
| Respiration | − | − | − | − | D± | D± | D± | D± | D± | D± | D± | D± | | |
| Salivation F.V. | − | − | − | − | − | − | − | − | − | − | − | − | | |
| Lacrimation | − | − | − | − | − | − | − | − | − | − | − | − | | |
| Diarrhea | − | − | − | − | − | − | − | − | − | − | − | − | | |
| Body Temperature | − | − | − | − | − | − | − | − | − | − | − | − | | |
| Piloerection | − | − | − | − | − | − | − | − | − | − | − | − | | |
| Inc. Palpebral Size | − | − | − | − | − | − | − | − | − | − | − | − | | |
| Dec. Palpebral Size | − | − | − | − | − | − | − | − | − | − | − | − | | |
| Others | − | − | − | − | E | E | E | E | E | E | E | E | | |
| Death | − | − | − | − | − | − | − | − | − | − | − | − | + | + |

Notes:
“−” no effects; “±”: Slight to moderate effects; Severe effects; “Inc.”: Increased; “Dec.”: Decreased; “Spont.”: Spontaneous; “C.”: Chronic; “T.”: Tonic; “C-T”: Chronic-Tonic; “F.”: Fluid; “V.:: Viscosity; “C.”: Cyanosis; “D.”: Depth; E: Edema

TABLE MTDR1
MTD results, mortality assessment prior and post dose administrations

| | | | Mortality | | | | |
|---|---|---|---|---|---|---|---|
| | | | (total number of deaths/total number of animals) | | | | |
| | | Dose | 5 min of | 1 h of | 5 min of | 1 h of | 5 min of |
| Compound | Route | (mg/kg) | 1^{st} dosing | 1^{st} dosing | 2^{nd} dosing | 2^{nd} dosing | 3^{rd} dosing |
| Vehicle | IV | 5 mL/kg, | 0/4 | 0/4 | 0/4 | 0/4 | 0/4 |
| (0.9% NaCl) | | TID, q8h | | | | | |
| PT# 1235860 | IV | 5 mg/kg, | 0/4 | 0/4 | 0/4 | 0/4 | 0/4 |
| (NID-162) | | TID, q8h | | | | | |
| (D-PLEUROCIDIN-KR) | | 10 mg/kg, | 0/4 | 1/4 | 1/4 | 1/4 | 1/4 |
| | | TID, q8h | | | | | |
| | | 30 mg/kg, | 4/4 | All dead | | | |
| | | TID, q8h | | | | | |
| | | | Mortality | | | | |
| | | | (total number of deaths/total number of animals) | | | | |
| | | Dose | 1 h of | 24 h of | 48 h of | 72 h of | |
| Compound | Route | (mg/kg) | 3^{rd} dosing | 3^{rd} dosing | 3^{rd} dosing | 3^{rd} dosing | |
| Vehicle | IV | 5 mL/kg, | 0/4 | 0/4 | 0/4 | 0/4 | |
| (0.9% NaCl) | | TID, 8qh | | | | | |
| PT# 1235860 | IV | 5 mg/kg, | 0/4 | 0/4 | 0/4 | 0/4 | |
| (NID-162) | | TID, q8h | | | | | |
| (D-PLEUROCIDIN-KR) | | 10 mg/kg, | 1/4 | 1/4 | 1/4 | 1/4 | |
| | | TID, q8h | | | | | |
| | | 30 mg/kg, | All dead | | | | |
| | | TID, q8h | | | | | |

*Note:
The 2^{nd} and 3^{rd} doses of the surviving animals in the 10 mg/kg group were not administered due to the mortality after the 1^{st} dose.

TABLE MTDR2
MTD results, body weight records prior and post dose administrations

| | | | | Body Weight (g) | | | | | |
|---|---|---|---|---|---|---|---|---|---|
| | | Dose | | | | | 24 h of | 48 h of | 72 h of |
| Compound | Route | (mg/kg) | No. | 1^{st} dosing | 2^{nd} dosing | 3^{rd} dosing | 3^{rd} dosing | 3^{rd} dosing | 3^{rd} dosing |
| Vehicle | IV | 5 mL/kg, | 1 | 25 | 25 | 25 | 26 | 26 | 26 |
| (0.9% NaCl) | | TID q8h | 2 | 24 | 24 | 25 | 25 | 25 | 26 |
| | | | 3 | 24 | 22 | 23 | 23 | 24 | 24 |
| | | | 4 | 24 | 24 | 25 | 25 | 25 | 26 |
| PT #1235860 | IV | 5 mg/kg, | 1 | 25 | 25 | 25 | 26 | 25 | 26 |
| (NID-162) | | TID q8h | 2 | 25 | 25 | 24 | 25 | 25 | 26 |
| (D-PLEUROCIDIN-KR) | | | 3 | 23 | 23 | 23 | 23 | 23 | 24 |
| | | | 4 | 25 | 25 | 24 | 25 | 25 | 26 |
| | | 10 mg/kg, | 1 | 22 | 21 | 21* | 21* | 21* | 22* |
| | | TID q8h | 2 | 23 | dead | | | | |
| | | | 3 | 23 | 23 | 23* | 24* | 24* | 23* |
| | | | 4 | 22 | 21 | 21* | 21* | 21* | 22* |
| | | 30 mg/kg, | 1 | 21 | dead | | | | |
| | | TID q8h | 2 | 22 | dead | | | | |
| | | | 3 | 23 | dead | | | | |
| | | | 4 | 24 | dead | | | | |

*Note:
The 2^{nd} and 3^{rd} doses of the surviving animals in the 10 mg/kg group were not administered due to the mortality after the 1^{st} dose.

TABLE of
Sequences

| | |
|---|---|
| SEQ ID NO:1 | Pleurocidin (P) |
| SEQ ID NO:2 | Pleurocidin-KR (PKR); D-Pleurocidin-KR |
| | (DPKR) when made of D amino acids |
| SEQ ID NO:3 | Pleurocidin-VA (PVA); D-Pleurocidin-VA |
| | (DPVA) when made of D amino acids |
| SEQ ID NO:4 | Pleurocidin-KRVA (PKRVA) D-Pleurocidin- |
| | KRVA (DPKRVA) when made of D amino acids |
| SEQ ID NO: 5 | See Table P |
| To | |
| SEQ ID NO: 15 | |
| SEQ ID NO: 16 | See Table V |
| To | |
| SEQ ID NO: 23 | |
| SEQ ID NO: 24 | Pleurocidin X1 to X11 |

Although illustrative embodiments of the invention have been disclosed in detail herein, with reference to the accompanying drawings, it is understood that the invention is not limited to those precise embodiments and that various changes and modifications can be effected therein by one skilled in the art without departing from the scope of the invention as defined by the appended claims and their equivalents.

1. A peptide comprising amino acid sequence having at least 52% sequence identity to SEQ ID NO: 1,
wherein said amino acid sequence comprises one or more of the following substitutions relative to SEQ ID NO: 1:
i. K7R
ii. K8R
iii. V12A or V12I or V12L
iv. K14R
v. V16A or V16I or V16 L
vi. K18R.

2. A peptide according to claim 1 comprising at least the following substitutions relative to SEQ ID NO: 1:
i. K7R
ii. K8R
iv. K14R; and
vi. K18R.

3. A peptide according to claim 1 comprising at least the following substitutions relative to SEQ ID NO: 1:
iii. V12A or V12I or V12L
v. V16A or V16I or V16 L.

4. A peptide according to claim 1 or claim 2 or claim 3 comprising at least the following substitutions relative to SEQ ID NO: 1:
i. K7R, and
ii. K8R, and
iii. V12A or V12I or V12L, and
iv. K14R, and
v. V16A or V16I or V16 L; and
vi. K18R.

5. A peptide according to any preceding claim further comprising one or more of the following substitutions relative to SEQ ID NO: 1:
a) F5Y or F5W
b) F6Y or F6W
c) substitution of H11 for 3-methylhistidine or 1-methylhistidine or 2,3-diaminopropionic acid or (N-methyl or N,N-dimethyl derivative of 2,3-diaminopropionic acid) or E or D;
d) substitution of H15 for 3-methylhistidine or 1-methylhistidine or 2,3-diaminopropionic acid or (N-methyl or N,N-dimethyl derivative of 2,3-diaminopropionic acid) or E or D
e) substitution of H23 for 3-methylhistidine or 1-methylhistidine or 2,3-diaminopropionic acid or (N-methyl or N,N-dimethyl derivative of 2,3-diaminopropionic acid) or E or D.

6. A peptide according to any preceding claim wherein one or more of amino acids 9 to 21 is substituted for N-substituted glycine.

7. A peptide according to any preceding claim wherein said peptide comprises amino acid sequence having at least 76% sequence identity to SEQ ID NO: 1.

8. A peptide according to any preceding claim wherein said peptide is at least 25 amino acids in length, suitably wherein said peptide is 25 amino acids in length.

9. A peptide comprising amino acid sequence selected from SEQ ID NO: 2 or SEQ ID NO: 3 or SEQ ID NO: 4, suitably consisting of amino acid sequence selected from SEQ ID NO: 2 or SEQ ID NO: 3 or SEQ ID NO: 4.

10. A peptide according to any preceding claim consisting of L-amino acids.

11. A peptide according to any preceding claim consisting of D-amino acids.

12. A peptide according to any of claims 1 to 11 for use in medicine.

13. A peptide according to any of claims 1 to 11 for use in treatment or prevention of infection, suitably bacterial infection.

14. A peptide according to any of claims 1 to 11 for use as an antimicrobial.

15. Use of a peptide according to any of claims 1 to 11 as an antimicrobial.

16. A pharmaceutical composition comprising a peptide according to any of claims 1 to 11.

17. A pharmaceutical composition according to claim 16 further comprising an antibiotic agent.

18. A method of treating or preventing infection, suitably bacterial infection, in a subject comprising administering a peptide according to any of claims 1 to 11 to said subject.

19. A method according to claim 18 further comprising administering an antibiotic agent to said subject.

20. A pharmaceutical composition according to claim 17 or a method according to claim 19 wherein said antibiotic agent comprises one or more of colistin, tobramycin and rifampin.

21. A pharmaceutical composition according to claim 17 or claim 20, or a method according to claim 19 or claim 20, wherein the bacterial infection is P.aeruginosa infection or A.baumannii infection, suitably P.aeruginosa infection.

22. A pharmaceutical composition according to claim 17 or claim 20 or claim 21, or a method according to claim 19 or claim 20 or claim 21, wherein the peptide comprises the amino acid sequence of SEQ ID NO:2, and consists of D-amino acids.
